(19) United States
(12) Patent Application Publication
(10) Pub. No.: US 2025/0195245 A1
(43) Pub. Date: Jun. 19, 2025

(54) NEUROVASCULAR IMPLANTS AND DELIVERY SYSTEMS

(71) Applicant: Imperative Care, Inc., Campbell, CA (US)

(72) Inventors: Jason Lee, Seattle, WA (US); Lilip Lau, Los Altos, CA (US); Sean Totten, Kirkland, WA (US); James H. Silver, Palo Alto, CA (US); Madeline Louise DeSenna, Campbell, CA (US)

(21) Appl. No.: 18/712,166

(22) PCT Filed: Nov. 21, 2022
(86) PCT No.: PCT/US2022/050609

§ 371 Date: May 21, 2024

Related U.S. Application Data
(60) Provisional application No. 63/281,923, filed on Nov. 22, 2021.

(51) Int. Cl. A61F 2/90; A61L 31/10; A61L 31/16; A61L 31/18; A61L 33/00; A61L 33/08

(52) U.S. Cl. CPC A61F 2/90; A61L 31/10; A61L 31/16; A61L 31/18; A61L 33/0011; A61L 33/08; A61F 2250/0098; A61L 2300/236; A61L 2300/42; A61L 2400/12

(57) ABSTRACT

Disclosed are implants, devices and systems capable of being deployed within the neurovasculature of a subject. The implants are configured for enhanced conformability to a vessel wall and have thromboresistant design features and coatings. The devices for implant deployment are configured for precise placement of an implant, resheathing of a partially deployed implant, and reliable detachment of an implant without distorting the positioning of the implant.

### CROSS-REFERENCE TO RELATED APPLICATIONS

This application claims priority to U.S. Provisional Patent Application No. 63/281,923, filed Nov. 22, 2021, titled “NEUROVASCULAR DEVICES HAVING THREE DIMENSIONAL CONFIGURATIONS AND SURFACE CHEMISTRIES FOR ENHANCED THROMBORESISTANCE AND/OR ENDOTHELIALIZATION,” the disclosure of which is hereby incorporated by reference in its entirety.

### TECHNICAL FIELD

The present disclosure relates to devices, systems, and methods for treating vascular disease, including devices, systems, and methods for controllably and selectively delivering a stent implant into a patient's vasculature.

### BACKGROUND

The mammalian circulatory system is comprised of a heart, which acts as a pump, and a system of blood vessels which transport the blood to various points in the body. Due to the force exerted by the flowing blood on the blood vessels, they may develop a variety of vascular defects. One common vascular defect known as an aneurysm is formed as a result of the weakening of the wall of a blood vessel and subsequent ballooning and expansion of the vessel wall. If an aneurysm is left without treatment, the blood vessel wall gradually becomes thinner and damaged, and, at some point, may be ruptured due to a continuous pressure of blood flow. Neurovascular or cerebral aneurysms affect about 5% of the population. In particular, a ruptured cerebral aneurysm leads to a cerebral hemorrhage, thereby resulting in a more serious life-threatening consequence than any other aneurysm, as cranial hemorrhaging could result in death.

Cerebral aneurysms may be treated by highly invasive techniques which involve a surgeon accessing the aneurysm through the cranium and possibly the brain to place a ligation clip around the neck of the aneurysm to prevent blood from flowing into the aneurysm.

A less invasive therapeutic procedure involves the delivery of embolization materials or devices into an aneurysm. The delivery of such embolization materials or devices may be used to promote hemostasis or fill an aneurysm cavity entirely. Embolization materials or devices may be placed within the vasculature of the human body, typically via a microcatheter, either to block the flow of blood through a vessel with an aneurysm through the formation of an embolus or to form such an embolus within an aneurysm stemming from the vessel. A variety of coil embolization devices are known. Coils are generally constructed of a wire, usually made of a metal (e.g. platinum) or metal alloy that is wound into a helix. The coils of such devices may themselves be formed into a secondary coil shape, or any of a variety of more complex secondary shapes. Coils are commonly used to treat cerebral aneurysms but suffer from several limitations including poor packing density, compaction due to hydrodynamic pressure from blood flow, poor stability in wide-necked aneurysms and complexity and difficulty in the deployment thereof as most aneurysm treatments with this approach require the deployment of multiple coils.

A variety of implants such as stents can be delivered via microcatheter to a vascular site of a patient, such as an aneurysm, to help retain embolic material or coils within the aneurysm, divert blood flow and/or retain patency of the vascular lumen. Typically, the implant is releasably retained on a distal end of either the delivery microcatheter or a guidewire contained within the microcatheter, and controllably released therefrom into the vascular site to be treated. The clinician delivering the implant must navigate the microcatheter or guide catheter through the vasculature and, in the case of intracranial treatment sites, navigation of the microcatheter is through tortuous microvasculature. This delivery may be visualized by fluoroscopy or another suitable means. Detachment may occur through a variety of means, including, electrolytic detachment, chemical detachment, mechanical detachment, hydraulic detachment, and thermal detachment. Once the microcatheter has positioned the mounted implant at the desired vascular deployment site, the clinician will seek to detach the implant from the catheter or guidewire without distorting the positioning of the implant.

Each of the various existing implant detachment/delivery technologies has strengths and weaknesses. For example, one mechanical deployment system involves proximal retraction of an outer sleeve to expose a self-expanding stent implant restrained by the sleeve. Unfortunately, the stent may prematurely deploy as the outer tube is partially retracted, and the exposed portion of the stent expands resulting in the stent being propelled distally beyond a desired deployment site. Also, once the stent has been partially unsheathed, it may sometimes be determined that the stent placement needs to be adjusted. With existing systems, the stent has a tendency to force itself out of the sheath and touch down against the vessel wall thereby making adjustments or resheathing of the stent difficult or impossible. Additionally, existing stents typically have one or more free apices or structural portions that can embed within tissue even in a partially-deployed state, further making adjustments or resheathing of the stent difficult or impossible.

While stents can be helpful to retain embolic material or coils within an aneurysm, stent implants themselves can introduce their own complications. Perhaps the main complication of a stent implant is the promotion of thrombosis formation due to the presence of the stent itself, with the resulting risk of embolization and stroke. Incomplete stent apposition, or a lack of contact between the structure of the stent and the underlying vessel wall not overlying a side branch, is another factor that can promote thrombosis with the use of stent implants. In the tortuous microvasculature of intracranial treatment sites, attaining complete stent apposition can be a challenge. Another complication from the use of covered stents or stent-grafts comprising a sleeve of polymeric material around the stent lumen is the potential to inadvertently occlude small perforating or branching vessels proximate the aneurysm.

Further, neurovascular devices may also be indicated for the treatment of intracranial artery stenosis (ICAS). ICAS accounts for about 10% of ischemic stroke cases. However, the incidence rates vary based on ethnicity: 5-10% of strokes in white population; 15-29% of strokes in black population; 30-50% of strokes in Asian population. ICAS-derived stroke results from three mechanisms: a) artery-to-artery embolism; b) hypoperfusion; c) plaque extension into and occlusion of perforators. Approximately 67% of ICAS occurs in non-basilar anatomy (intracranial and extracranial ICA etc.) and the other ˜33% of ICAS occurs in basilar artery. Peri-procedural risk in ICAS stenting is significant, primarily through perforator occlusion. Plaque rupture is also possible but is unconfirmed because ICAS pathobiology is less understood (or studied) compared to coronary lesions. Angioplasty (including stenting) in basilar artery ICAS has greater peri-procedural risk because of abundant perforators. Restenosis does occur in angioplasty (including stenting) cases in longer timeframes. SAAMPRIS, WASID, WARSS trials provide hypotheses-generating insights into mechanisms of clinical events; however, an effective therapy is yet to be realized. Thus, treatment of ICAS also presents a significant clinical need for improved neurovascular implants.

Despite prior efforts there remains a need for improved intraluminal stent implants as well as improved detachment/delivery devices.

### SUMMARY

Provided herein are thromboresistant stent implants having hemodynamically enhanced geometry, enhanced conformability to maximize implant-to-vessel wall apposition, and/or thromboresistant coatings. Also provided herein are delivery devices that can allow exact placement of stent implants, resheathing of partially exposed stent implants, and reliable detachment of stent implants without distorting the positioning of the stent implants.

The implants described herein may be permanently implantable, deployable and retrievable, or part of an interventional catheter or other transient intravascular device. In neurovascular applications the implant may be an aneurysm bridge or other implant relating to prevention or treatment of stroke. For example, the implants described herein can be used for stent assisted coiling of wide neck aneurysms, treating intra-cranial atherosclerotic stenosis, or maintenance of flow in acute ischemic stroke in conjunction with thrombectomy. The implants described herein can also be used in other vessels and/or vasculature of the body, such as for the treatment and/or prevention of an aneurysm, vascular stenosis, heart disease, artery disease, deep vein thrombosis, or other conditions.

The combinations of hemodynamic geometry with surface modification disclosed herein produces a thrombo-embolism resistant implant over a range of flow rates from about 5 ml/min to about 400 ml/min. The combination should minimize or prevent all types of thrombi (red thrombus, white thrombus, mixed thrombi) and white cells-thrombi combinations by targeting multiple mechanisms of thrombus formation. In addition, the implants described herein having combined geometry and surface modification can be both thrombus resistant at the implant site and resistant to distal emboli shedding away from the implant site. In some implementations, the implants elicit a faster rate of functional endothelialization.

The implant geometry may be optimized for load-bearing function; anatomical compliance; fluid dynamic interaction for low platelet activation; and/or ease of procedural deployment. The implant surface may be engineered to: modulate and prevent adverse interactions between the implant surface and blood platelets and/or culprit proteins; and prevent platelet activation in the vicinity of the implant, beyond the surface by interacting with both surface-contacting and near-wall excess platelet population.

Disclosed herein is a self-expanding thromboresistant intraluminal implant, the implant comprising a generally tubular frame. The generally tubular frame can comprise a proximal portion, a distal portion, and a central portion. The proximal portion can comprise a ring that extends along a circumference of the tubular frame, the ring comprising a plurality of ring struts, wherein adjacent pairs of ring struts join at a plurality of proximal apexes and a plurality of distal apexes to form a chevron pattern. The distal portion can comprise a ring that extends along the circumference of the tubular frame, the ring comprising a plurality of ring struts, wherein adjacent pairs of ring struts join at a plurality of proximal apexes and a plurality of distal apexes to form a chevron pattern. The central portion can be disposed between the proximal portion and the distal portion, the central portion comprising a plurality of longitudinally spaced apart rings that extend along the circumference of the tubular frame, each ring of the plurality of rings comprising a plurality of rings struts, wherein adjacent pairs of ring struts join at a plurality of proximal apexes and a plurality of distal apexes to form a chevron pattern, and a plurality of linking struts that extend at least partially along the circumference of the tubular frame, each linking strut of the plurality of linking struts connecting a distal apex of one ring of the plurality of rings to a proximal apex of an adjacent ring of the plurality of rings.

In the above intraluminal implant or in other implementations as described herein, one or more of the following features can also be provided. In some implementations, each linking strut of the plurality of linking struts connect each one of the plurality of distal apexes of one ring of the plurality of rings of the central portion to each one of the plurality of proximal apexes of an adjacent ring of the plurality of rings of the central portion except for at each one of a plurality of distal apexes of a distal most ring of the central portion and except for at each one of a plurality of proximal apexes of a proximal most ring of the central portion such that the central portion does not comprise any free apexes. In some implementations, each distal apex of the plurality of distal apexes of the distal most ring of the central portion connects to a respective proximal apex of the plurality of proximal apexes of the ring of the distal portion, and wherein each proximal apex of the plurality of proximal apexes of the proximal most ring of the central portion connects to a respective distal apex of the plurality of distal apexes of the ring of the proximal portion. In some implementations, each distal apex of the plurality of distal apexes of the one ring of the plurality of rings of the central portion is rotationally offset from each proximal apex of the plurality of proximal apexes of the adjacent ring of the plurality of rings of the central portion such that at least a portion of each linking strut of the plurality of linking struts extends along a helical path at least partially around the circumference of the tubular frame. In some implementations, the at least a portion of each linking strut of the plurality of linking struts connecting each distal apex of the plurality of distal apexes of the one ring of the plurality of rings of the central portion to each proximal apex of the plurality of proximal apexes of the adjacent ring of the plurality of rings of the central portion extends along the helical path at least partially around the circumference of the tubular frame in a first helical direction, and wherein at least a portion of each linking strut of a plurality of linking struts connecting each distal apex of a plurality of distal apexes of the adjacent ring of the plurality of rings of the central portion to each proximal apex of a plurality of proximal apexes of another adjacent ring of the plurality of rings of the central portion extends along the helical path at least partially around the circumference of the tubular frame in a second helical direction that is generally opposite the first helical direction. In some implementations, the intraluminal implant further comprises one or more generally proximally extending struts extending from a respective one or more proximal apex of the plurality of proximal apexes of the ring of the proximal portion. In some implementations, each of the one or more generally proximally extending struts comprises a neck portion and a connection portion, the connection portion configured to connect to a radiopaque marker. In some implementations, the intraluminal implant further comprises one or more generally distally extending struts extending from a respective one or more distal apex of the plurality of distal apexes of the ring of the distal portion. In some implementations, each of the one or more generally distally extending struts comprises a neck portion and a connection portion, the connection portion configured to connect to a radiopaque marker. In some implementations, the intraluminal implant further comprises one or more radiopaque markers configured to connect to the one or more generally proximally extending struts and/or the one or more generally distally extending struts at the connection portion thereof. In some implementations, the proximal portion flares radially outward in a proximal direction. In some implementations, the distal portion flares radially outward in a distal direction. In some implementations, the plurality of linking struts do not overlap one another. In some implementations, the intraluminal implant is configured to have less malappositions between the intraluminal implant and an inner wall of a vessel in which it is deployed on an inside of a bend of the vessel than on an outside of the bend of the vessel. In some implementations, the central portion of the tubular frame has a diameter of about 3 mm. In some implementations, the intraluminal implant, when deployed centered inside a flexible silicone U-bent tube at a bend radius of 4.9 mm and having an inner diameter of 3 mm, has 16 or less malappositions with an inner wall of the U-bent tube. In some implementations, a maximum malapposed distance of the 16 or less malappositions is 0.400 mm or less. In some implementations, an average malapposed distance of the 16 or less malappositions is 0.120 mm or less. In some implementations, the central portion of the tubular frame has a diameter of about 4 mm. In some implementations, the implant has a length of between about 10 mm and about 50 mm. In some implementations, the tubular frame is cut from tubing that is about a same diameter of the central portion of the tubular frame. In some implementations, the implant does not include a graft, covering, or liner. In some implementations, the intraluminal implant further comprises a heparin coating.

Disclosed herein is a self-expanding thromboresistant intraluminal implant, the implant comprising a generally tubular frame comprising a plurality of longitudinally spaced apart rings that extend along a circumference of the tubular frame, each ring of the plurality of rings comprising a plurality of rings struts, wherein adjacent pairs of ring struts join at a plurality of proximal apexes and a plurality of distal apexes to form a chevron pattern, and a plurality of linking struts that extend at least partially along the circumference of the tubular frame, each linking strut of the plurality of linking struts connecting a distal apex of one ring of the plurality of rings to a proximal apex of an adjacent ring of the plurality of rings; wherein the tubular frame comprises a wall thickness of about 45 μm or less, and wherein the implant comprises a heparin coating.

In the above intraluminal implant or in other implementations as described herein, one or more of the following features can also be provided. In some implementations, the heparin coating has a thickness of about 30 nm or less. In some implementations, the heparin coating has a mass of about 1.0 ug or less. In some implementations, a ratio of a mass of the heparin coating to a total surface area of the implant is about 0.007 ug/mm² or more. In some implementations, a ratio of a mass of the heparin coating to the wall thickness of the tubular frame is about 0.007 μg/mm or more. In some implementations, a ratio of a mass of the heparin coating to an abluminal surface area of the implant is about 0.03 μg/mm² or more. In some implementations, a ratio of a thickness of the heparin coating to the wall thickness of the tubular frame is about 0.00016 or greater. In some implementations, a particle size equivalent to an entirety of the heparin coating is about 101 μm in diameter or less. In some implementations, a ratio of heparin activity of the heparin coating to the wall thickness of the tubular frame is about 0.80 pmol AT/cm²/μm or more. In some implementations, the tubular frame has a diameter of about 3 mm. In some implementations, the tubular frame has a diameter of about 4 mm. In some implementations, the implant has a length of between about 10 mm and about 50 mm. In some implementations, the implant does not include a graft, covering, or liner.

Disclosed herein is a method of stenting a vessel of a patient, the method comprising using the intraluminal implant, delivery device and/or system of the foregoing description.

Disclosed herein is a system comprising one or more features of the foregoing description.

Disclosed herein is an implant comprising one or more features of the foregoing description.

Disclosed herein is an intraluminal delivery device comprising one or more features of the foregoing description.

Disclosed herein is a method of treating a patient's vasculature comprising one or more features of the foregoing description.

For purposes of summarizing the disclosure, certain aspects, advantages and novel features of several implementations have been described herein. It is to be understood that not necessarily all such advantages are achieved in accordance with any particular implementation of the technology disclosed herein. Thus, the implementations disclosed herein can be implemented or carried out in a manner that achieves or optimizes one advantage or group of advantages as taught herein without necessarily achieving other advantages that can be taught or suggested herein.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A illustrates insertion of a microcatheter through the groin and into the neurovascular region of a patient in accordance with some aspects of this disclosure.

FIG. 1B illustrates potential treatments sites of the basilar and non-basilar anatomy of a patient in accordance with some aspects of this disclosure.

FIGS. 2A-2E illustrate a self-expanding thromboresistant intraluminal implant in accordance with some aspects of this disclosure.

FIG. 3 illustrates an intraluminal implant of FIGS. 2A-2E in an apposition bend test in accordance with some aspects of this disclosure.

FIG. 4 illustrates a variant of the intraluminal implant of FIGS. 2A-2E in accordance with some aspects of this disclosure.

FIG. 5 illustrates a variant of the intraluminal implant of FIGS. 2A-2E in accordance with some aspects of this disclosure.

FIG. 6 illustrates a delivery wire in accordance with some aspects of this disclosure.

FIGS. 7A-7B illustrate a core wire of the delivery wire of FIG. 6 in accordance with some aspects of this disclosure.

FIGS. 8A-8D illustrate a bumper of the delivery wire of FIG. 6 in accordance with some aspects of this disclosure.

FIGS. 9A-9E illustrate a coupler of the delivery wire of FIG. 6 in accordance with some aspects of this disclosure.

FIGS. 10A-10B illustrate details of the delivery wire of FIG. 6 in accordance with some aspects of this disclosure.

FIGS. 11A-11I illustrate an intraluminal implant delivery system in accordance with some aspects of this disclosure.

FIGS. 12A-12C illustrate delivery of an intraluminal implant in accordance with some aspects of this disclosure.

FIGS. 13Aa-13Gb illustrate delivery of an intraluminal implant adjacent an aneurysm in accordance with some aspects of this disclosure.

FIGS. 14A-14F illustrate another implementation of an intraluminal implant delivery system in accordance with some aspects of this disclosure.

FIGS. 15A-15E illustrate delivery of an intraluminal implant in accordance with some aspects of this disclosure.

FIGS. 16A-16D illustrate implementations of radiopaque markers in accordance with some aspects of this disclosure.

FIGS. 17A-17G illustrate a method of treating an aneurysm in accordance with some aspects of this disclosure.

FIG. 17H illustrates a variant of the method of treating an aneurysm of FIGS. 17A-17G in accordance with some aspects of this disclosure.

FIGS. 18A-18B illustrates an introducer sheath in accordance with some aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

### Overview

The present disclosure describes various implementations of intraluminal implants (e.g., stent implants), intraluminal implant delivery devices, intraluminal implant systems, and methods of implanting an intraluminal implant. Such implants, devices, systems, and methods can be used to stent a vessel of a patient, such as a vessel in the neurovasculature of a patient. Furthermore, such implants, devices, systems, and methods can be used to treat an aneurysm of a patient, such as a neurovascular aneurysm, and/or treat intracranial and/or extracranial artery stenosis. The implants, devices, systems, and methods disclosed herein can advantageously allow for the exact placement of an implant in a patient's vessel, resheathing of a partially exposed or deployed implant, and/or reliable detachment of an implant without distorting the positioning of the implant. Furthermore, the implants, devices, systems, and methods disclosed herein can advantageously provide a thromboresistant intraluminal implant. For example, the intraluminal implants disclosed herein can be configured to maximize implant-to-vessel wall apposition and minimize implant-to-vessel wall malapposition, which can advantageously prevent and/or reduce areas of stagnant or low flow of bodily fluid (e.g., blood) through the vessel in which the implant is located. Such a configuration can be particularly advantageous in the tortuous microvasculature of intracranial treatment sites, which can have vessels of small diameter with tight bends. As another example, the intraluminal implants disclosed herein can be configured to have a thromboresistant coating, such as a heparin coating. In another example, the intraluminal implants disclosed herein can be configured to have little to no impact on bodily fluid, such as blood, flowing therethrough after implantation. Additionally, the implants disclosed herein can advantageously be configured to prevent and/or limit occlusion of small perforating or branching vessels proximate the site of the implant. For example, the intraluminal implants disclosed herein can have a frame without a graft/sleeve that would prevent and/or limit the flow of bodily fluid through the frame of the implant.

The intraluminal implants, devices, systems and methods described herein can be adapted for percutaneous delivery. As such, the intraluminal implants described herein can be configured to be delivered via a delivery device as described herein (e.g., a catheter-based delivery device) and can have a collapsed configuration for delivery into a patient and can expand from the collapsed configuration to an expanded configuration for implantation within the patient. For example, the intraluminal implants described herein can be self-expanding with an expansion ratio of at least about 2:1, at least about 3:1, at least about 4:1, at least about 5:1, at least about 6:1, at least about 7:1, or at least about 8:1. In some implementations, the implants, devices and systems described herein can be configured for implantation within the patient's vasculature. For example, an intraluminal implant as described herein can be percutaneously implanted via a delivery device as described herein through an artery of a patient to an intracranial delivery site within the patient. Such an intraluminal implant can stent a vessel of the patient at the intracranial delivery site and allow blood flow therethrough. Delivery may be through a catheter/microcatheter or a guidewire lumen of a PTCA balloon. Target treatment arteries can include Anterior Distal-M1, M2, M3, Acom, ACA, Posterior, basilar, Pcom, among others as described herein. Delivery may be remote, and may be robotically controlled. Delivery may be accomplished with 2 catheters (microcatheter, 0.088 guide) rather than three.

The intraluminal implants, devices and systems described herein can be sized and configured for implanting an implant within a target vessel of interest of a patient. For example, the intraluminal implants, devices, and systems described herein can be sized and configured for implanting an implant within any intracranial vessels such as an anterior cerebral artery, internal carotid artery, basilar artery, anterior inferior cerebellar artery, middle cerebral artery, posterior inferior cerebellar artery, vertebral artery, anterior communicating artery, posterior cerebral artery, posterior communicating artery, lenticulostriate arteries, internal carotid artery, or any one or more of the branches thereof. As another example, the intraluminal implants, devices, and systems described herein can be sized and configured for implanting an implant within any cardiac vessels such as an infundibular vein, anterior cardiac veins, right marginal vein, small cardiac vein, great cardiac vein, anterior interventricular vein, septal veins, oblique vein of Marshall, left marginal vein, left posterior veins, left atrial vein, posterior interventricular vein, acute marginal artery, left circumflex artery, left anterior descending artery, septal artery, conus branch, SA nodal branch, left circumflex artery, obtuse marginal artery, posteriolateral branch, right coronary artery, posterior descending artery, or any one or more of the branches thereof.

An intraluminal implant, which can also be referred to herein as an implant, a stent, and/or a stent implant, can have an expanded (e.g., implanted) diameter in the range of about 1 mm to about 6 mm, about 2 mm to about 5 mm, about 3 mm to about 4 mm, or it can have a diameter greater than about 1 mm or less than about 6 mm depending on the application. In some implementations, an implant as described herein can have an unconstrained expanded diameter in the range of about 1 mm to about 6.5 mm, about 2 mm to about 5.5 mm, about 3 mm to about 4.5 mm, or it can have a diameter greater than about 1 mm or less than about 6.5 mm depending on the application. An implant as described herein can be oversized for the vessel of interest and thus impart an outward force on the vessel in which it is implanted (e.g., to improve anchoring within the vessel). An implant can have an expanded (e.g., implanted) length in the range of about 5 mm to about 50 mm, about 5 mm to about 45 mm, about 5 mm to about 40 mm, about 5 mm to about 35 mm, about 5 mm to about 30 mm, about 10 mm to about 30 mm, about 10 mm to about 25 mm, about 15 mm to about 23 mm, or it can have a length greater than about 5 mm or less than about 50 mm depending on the application.

The intraluminal implants described herein configured for implantation within a vessel of a patient can include a generally tubular and expandable frame (configured for percutaneous delivery as described herein) with a thromboresistant coating. The tubular frame can have a proximal end, a distal end, and a lumen extending from the proximal end to the distal end. The tubular frame can generally comprise a plurality of rings that extend along a circumference of the tubular frame, with adjacent rings generally connected to one another by a plurality of linking struts. The ring struts and linking struts of the frame can be configured to provide an intraluminal implant with enhanced flexibility and conformability. Furthermore, the tubular frame can be generally devoid of free apices along a central portion of the tubular frame to aid in the ability of the implant to be resheathed and/or repositioned after partial deployment of the implant.

The tubular body can be made of a material configured to expand upon delivery, and as such can comprise a shape memory material such as nitinol. In some implementations, the expandable body can be configured to radially collapse/crimp. In some variations, the expandable body can comprise a material without or with little shape memory, and a balloon can be used to expand the expandable body for implantation. Such a balloon can be an occlusive balloon or a non-occlusive balloon, such as a hollow balloon. The intraluminal implant can include one or more coatings, such as one or more antithrombotic coatings and/or one or more drug-eluting coatings. In some implementations, an implant can comprise a drug-eluting implant for treatment of ICAD/ICAS, for example with anti-restenotic properties and/or in the setting of acute stroke. In some implementations, it is desirable to utilize a material and/or coating to prevent ingrowth within the implant to aid in later implant retrieval and/or removal. Conversely, in some cases it is desirable to utilize a material and/or coating to allow and/or promote ingrowth within the implant and/or around the frame and any of struts or radiopaque markers of the implant.

Vascular access for the delivery of an intraluminal implant as described herein can include an internal jugular vein, a subclavian vein, a femoral vein, and/or others. From such access points, an implant can be advanced within the patient's vasculature by a delivery device (e.g., a delivery catheter) as described herein until the desired location of implantation is reached, thereupon the implant can be delivered and expanded for implantation. An introducer sheath, a guidewire, a guide catheter, an access catheter, and/or other devices or components can be utilized for delivery, as well as standard imaging methods. Furthermore, the implants and associated delivery devices described herein can include radiopaque features to aid in delivery and implantation.

One or more intraluminal implants as described herein can be implanted within a patient. In some cases, it can be beneficial to have only one intraluminal implant implanted within a patient, or it can be beneficial to have multiple intraluminal implants implanted within a patient. If multiple intraluminal implants are implanted within a patient, such implants can work together as needed to achieve the treatment outcome desired. Furthermore, intraluminal implants of the same or different sizes can be implanted within the same patient.

In any of the implementations described herein, an implantable device may be configured and/or coated for use in treatment of aneurysms and/or ICAS.

The implant coating may inhibit or substantially inhibit thrombus formation (e.g, the coating can be thromboresistant). In some implementations the implant geometry and/or coating can promote or substantially prevent endothelialization. Thromboresistance may be achieved, for instance, by reduction of protein adsorption, cellular adhesion, and/or activation of platelets and coagulation factors (e.g., low platelet stress accumulation (δdt). Endothelialization may be accomplished by promoting the migration and adhesion of endothelial cells from the intimal surface of a native blood vessel wall or from circulating endothelial progenitor cells onto the implant and/or by the seeding of endothelial cells on the implant prior to implantation. In preferred implementations, the coating may be thin, robust (e.g. does not flake off with mechanical friction), and/or adheres to metallic surfaces such as nitinol, cobalt chromium, stainless steel, etc. The coating properties may be achieved by selection of the coating material, processing of the coating on the implant, and/or design of the coating surface. In some implementations, implant geometry may be optimized to achieve a low amount of platelet stress accumulation while maintaining other load bearing properties of the implant.

In some implementations, a coating for an implant can include a passive thromboembolism-resistant coating, such that the coating interacts at the implant surface with proteins and blood components or factors (e.g., platelets, cells, etc.). As will be described elsewhere herein, exemplary, non-limiting implementations of passive thromboembolism-resistant coatings include: poly(vinylidene fluoride co-hexafluoropropylene) (PVDF-HFP), fluorophosphazenes, heparin-polyvinylpyrrolidone-poly(ethylene glycol) (HEP-PVP-PEG), and phosphorylcholine-polyvinylpyrrolidone (PC-PVP).

In some implementations, a coating for an implant can include an active thromboembolism-resistant coating, such that the coating interacts at the implant surface and/or in the near surface region with proteins and blood components or factors (e.g., platelets, cells, etc.). An active thromboembolism-resistant coating can include a locally eluting system and/or a coating configured to capture (e.g., interact or bind with surface receptors) proteins and/or blood components, for example endothelial progenitor cells (EPCs).

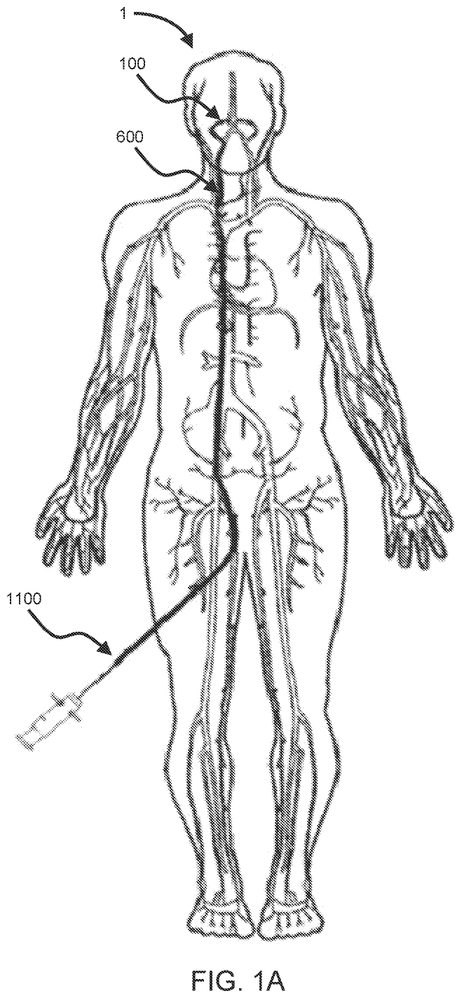

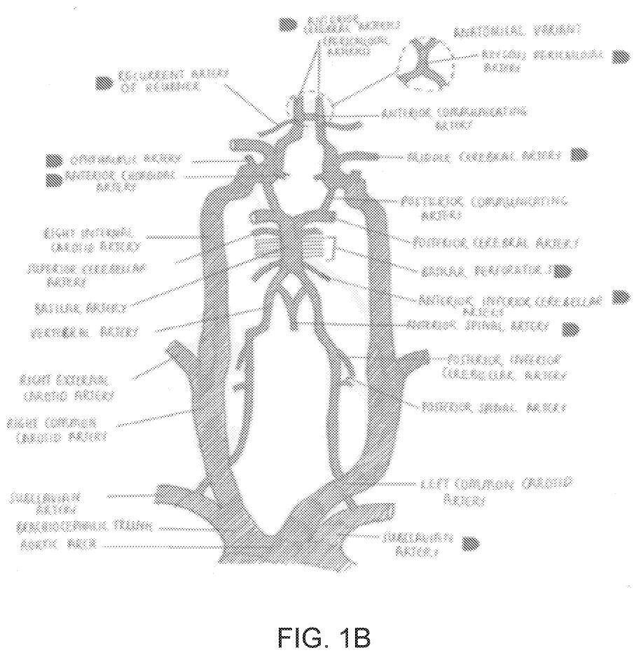

In some implementations, the implant coating can reduce peri-procedural risk and/or immediate post-procedural risk during treatment of ICAS. In some implementations, an implant for treating ICAS is configured for insertion into non-basilar anatomy, as shown in FIG. 1B. In some implementations, an implant for treating ICAS is configured for insertion into basilar anatomy, as shown in FIG. 1B. In some implementations, an implant for treating ICAS is configured to stabilize a plaque, reduce rupture or rupture potential, and/or prevent restenosis. In some implementations, an implant for treating ICAS is configured for use with dual antiplatelet therapy (DAPT) or single antiplatelet therapy (SAPT).

A coating material may be selected from, derived from, partially composed of, or produced from a combination of a number of materials, including but not limited to: fluorinated or perfluorinated polymers (e.g, polyvinylidene fluoride (PVDF) or copolymers thereof, fluorophosphazenes, etc.); plasma-deposited fluorine materials; zwitterionic substances; polyvinylpyrrolidone (PVP); phosphorylcholine (PC); poly(butyl methacrylate) (PBMA); polydimethyl siloxane (PDMS); albumin; glycosaminoglycan (GAG); sulfonated materials; glyme materials; polyethylene glycol (PEG)-based materials; carboxybetaine, sulfobetaine, or methacrylated versions thereof; self-assembled monolayers (e.g., fluorosilanes); heparin or heparin-like molecules or other anticoagulants; direct thrombin inhibitors (e.g., Hirudin, Bivalirudin, Lepirudin, Desirudin, Argatroban, Inogatran, Melagatran, ximelagatran, Dabigatran, etc.); curcumin; thrombomodulin; prostacyclin; DMP 728 (a platelet GPIIb/IIIa antagonist); chitosan or sulfated chitosan; hyaluronic acid; tantalum-doped titanium oxide; oxynitrides, oxide layers, inorganic materials such as diamond-like carbon (DLC) or fluorinated-DLC, and silicon carbide.

In some implementations, a coating comprises primarily heparin. The heparin coating may be created according to the methods described in U.S. Pat. No. 5,529,986, which is herein incorporated by reference in its entirety. Additionally, or alternatively, the heparin coating may be created using a photochemical crosslinker such as benzophenone according to the methods described in U.S. Pat. No. 7,550,444, which is herein incorporated by reference in its entirety. For example, a heparin coating may be applied to a polyvinylidene fluoride-co-hexafluoropropylene (PVDF-HFP) surface. The PVDF-HFP surface may be on a drug-eluting stent, for example, such that the heparin is applied over or under the PVDF-HFP surface. For example, polyethylene imine (PEI) is adsorbed from a solution (either from pure PEI or PEI diluted in water, methanol, ethanol or chloroform) onto the PVDF coating. A macromolecular complex of heparin with polylysine is formulated and applied to the PEI layer, as described in U.S. Pat. No. 5,529,986, which is herein incorporated by reference in its entirety. Chemisorption occurs and binds the heparin to the surface through one or more or a plurality of ionic interactions.

In some implementations, a heparin coating may be thin, for example in a range of from about 1 nm to about 1 micrometer, preferably less than 1 micrometer or less than 100 nm or less than 50 nm or less than 15 nm or less than 10 nm, as measured by transmission electron microscope focused ion beam (TEM-FIB). In some implementations, the heparin coating may be in a range of between about 5 nm to about 15 nm, about 5 nm to about 12 nm, about 4 nm to about 13 nm, preferably between about 5.4 nm and about 12 nm, even more preferably between about 8 nm and about 9 nm, as measured by TEM-FIB.

In some implementations, when these coatings are applied to an implant comprising nitinol, reduced or absent light exposure is desirable. An electropolished nitinol surface comprises titanium oxide, which can act as a photocatalyst and degrade many organic molecules. An electropolished nitinol device has a surface layer that is substantially depleted in nickel ions and is an amorphous TiOₓ (see, e.g., Nagaraja, S and Pelton, A., “Corrosion resistance of a Nitinol ocular microstent: Implications on biocompatibility”, J. Biomed Mater Res. 2020; 108B: 2681-2690, which is herein incorporated by reference in its entirety). Photocatalysis by TiO₂ is most efficient when ultraviolet (UV) light (e.g., at a wavelength of about less than 413 nm or less than about 420 or less than about 415 nm or between about 315 nm to about 415 nm) is used. The irradiation of titanium dioxide in the presence of oxygen and water generates hydroxyl radical species which may degrade heparin and other organic species (see, e.g., Blazkova, A., et al, “Photocatalytic degradation of heparin over titanium dioxide” J. Materials Science 30 (1995) 729-733, the contents of which are herein incorporated by reference in their entirety). The hydroxyl radicals created during this process can cause the degradation of organic species, including heparin. Furthermore, this process may also result in the implant surface becoming more or relatively hydrophilic, which can potentially interfere with interactions between the implant surface and various primers or coatings, such as the primers and/or coatings described herein. Thus, in some implementations, it is advantageous to modify the surface hydrophobicity of the implant after processing, for example, to increase the hydrophobicity of the implant surface (e.g., to become more hydrophobic) to improve bonding thereof with a primer and/or coating.

To prevent the photodegradation of heparin and like species, light exposure during manufacturing, storage, transportation, and end use can be minimized. This may be done by storing the coated devices in polyimide tubes, or other opaque storage containers which block UV light.

In some implementations, the coating comprises primarily plasma-deposited fluorine to form a hydrophobic surface. The fluorine may be derived from fluorocarbon gases (plasma fluorination), such as perfluoropropylene (C₃F₆), and the precursor molecules may be cross-linked on the device surface to form a more robust coating.

In some implementations, the coating consists primarily of plasma-deposited glyme. Glyme refers to glycol ether solvents, which share the same repeating unit as poly(ethylene oxide) (PEO) and poly(ethylene glycol) (PEG), and therefore exhibits some of the same biological properties as materials derived from those polymers. The glyme may be derived from tetraglyme (CH₃O(CH₂CH₂O)₄CH₃), for example, and the precursor molecules may be cross-linked on the device surface to form a more robust coating.

In some implementations, the coating consists primarily of phosphorylcholine biomaterials. Phosphorylcholine is the hydrophilic polar head group of some phospholipids, including many that form bi-layer cell membranes on red blood cells. Phosphorylcholine is zwitterionic, comprising a negatively charged phosphate covalently bonded to a positively charged choline group. The high polarity of the molecule is believed to confer phosphorylcholine biomaterials with a strong hydration shell that resists protein absorption and cell adhesion. Phosphorylcholine is commonly employed in coating coronary drug-eluting stents to help prevent restenosis and resist thrombosis. Polymeric phosphorylcholine biomaterials may attach both hydrophobic domains as well as phosphorylcholine groups to a polymer chain, with the hydrophobic domains serving to anchor the polymer chains to the surface to be coated and the phosphorylcholine groups orienting themselves toward the aqueous biological environment. Phosphorylcholine biomaterials may be used to coat metals, including stainless steel, nitinol, titanium, gold, and platinum; plastics, including polyolefins, polyvinyl chloride (PVC), poly(methyl methacrylate) (PMMA), polyethylene terephthalate (PET), polyurethane (PU), polycarbonate, polyamides, polyimides, polystyrene, and polytetrafluoroetylene (PTFE); rubbers, including silicone, latex, and polyisobutylene (PIB); glasses; ceramics; and biological tissues such as tooth enamel. Phosphorylcholine-conjugated polymers may also be used to form bulk biomaterials, in which the polymeric backbone is cross-linked.

In some implementations, the polymer backbone may be a methacrylate polymer which incorporates phosphorylcholine. In many implementations, phosphoryl choline groups will comprise at least 1%, 5%, 10%, 15%, 20%, 25%, or more than 25% of the functional groups attached to the polymer backbone. These polymers may be produced synthetically, such that the molecular structure may be precisely controlled, but may still closely mimic naturally occurring biomolecules. Various monomers may be included in phosphorylcholine polymers which alter its precise chemical properties and may be useful for tailoring phosphorylcholine biomaterials for drug delivery by affecting the material's interaction with drug payloads. Water content, hardness, and/or elasticity can be easily modulated with phosphorylcholine biomaterials. Phosphorylcholine biomaterials coatings may be applied to surfaces through reliable and highly reproducible solution-based techniques and are relatively simple to sterilize. Suitable compositions of phosphorylcholine may include Vertellus' PC 1036 and/or PC 1059.

In some implementations, the coating comprises primarily fluorinated or perfluorinated polymers applied via solution-based processing. Like the plasma-deposited fluorine surface, the fluorinated or perfluorinated polymers result in a hydrophobic surface. To facilitate attachment, a primer such as poly n-butyl methacrylate (PBMA), which may preferably be between about 264 and 376 kDa, may be first applied to the implant. An appropriate polymer precursor may be poly(vinylidene fluoride co-hexafluoropropylene) (PVDF-HFP) and may preferably comprise molecular weights between about 254 and 293 kDa. The PVDF-HFP may be applied via a solvent with a low surface tension to facilitate spreading and preferably a solvent that evaporates quickly. The polymer solution may be applied by dip coating or a spin or drying technique. Applying heat drying or forced air to the freshly coated device may reduce webbing. The fluorinated or perfluorinated polymers may be cross-linked on the implant surface to produce a more robust coating. Other suitable fluoropolymers may include polyvinylidene fluoride (PVDF), fluorophosphazenes, fluorinated ethylene propylene, tetrafluoroethylene, hexafluoropropylene, fluorinated silanes (e.g., perfluoroundecanoyl silane).

Exemplary, non-limiting examples of coating combinations include: PC, PEG, heparin, and PVP; PC and PEG; heparin, PEG, and PVP; PC and PVP; PEG-co-PBMA-co-PEG; PC and PBMA; PEG and PBMA, either in random or block architecture. For example, one or more polymers may be added to increase adhesiveness to the metal surface of the implant (e.g., PBMA), to increase biomimicry of the implant (e.g., phosphorylcholine), increase protein repellence of the implant (e.g., PEG), etc. The ratio and/or branching (e.g., linear, branched, hyperbranched, comb-brush, multi-arm star, etc.) of the two or more polymers may be optimized for the type of condition (e.g., aneurysm, intracranial atherosclerotic disease, etc.), location, time after inciting injury or incident, etc.

In some implementations, a polymer for coating a device can include a terpolymer comprising PC-co-X-co-PEG in a random or block configuration, where X comprises a metal adherence group such as PBMA. For example, the polymer configuration can include: PC-X-PEG; X-PC-PEG (block); X-PEG-PC (block); or X-(PEG-PC-PEG-PC-PEG-PC-PEG-PC) (random). The branching structure (e.g., linear, branched, hyperbranched, comb-brush, multiarm star, etc.) of the polymer can also be optimized.

In some implementations, the coating can include surface modifying additives (SMA) that are block co-polymers that contain one block that is miscible with bulk polymer and the other block is a functional block that is immiscible with the bulk polymer and is added during thermal processing. In some implementations, SMA processing may be modified to further accelerate surface blooming through secondary processes such as temperature optimizing for slightly less than bulk polymer glass transition (Tg), but above SMA-Tg to minimize thermal property change of the bulk polymer while allowing SMA migration. In some implementations, the extruded or molded part forming tubes or wires can be exposed to a solvent environment that plasticizes the bulk polymer that can accelerate SMA migration. In some implementations, a good solvent (e.g., up to 80% solubility in the solvent) may be used for the SMA migration but a marginal solvent (e.g., no more than 0.5% solubility in the solvent) for the bulk polymer, so that the bulk polymer plasticizes to form tubes, wires, and/or films. Table 1 summarizes possible solvents for SMA migration, where X=insoluble, O=soluble, #=partially soluble, and *=unknown. The solvent exposure may be direct contact or a solvent-humid environment with solvent exposure time duration in a fixed or cyclic on-off time. In some implementations, the SMA and bulk polymer can be electrosprayed, electrospun, or solution spun to form tubes, wires, and/or films.

TABLE 1
Solvents for SMA Migration

| Good solvents for | | | | |
|---|---|---|---|---|
| SMA migration, | | | | |
| PVDF HFP | PVP | PNIPAAM | CMC | PEBAX |
| Acetone | X | ◯ | ◯ | X |
| Methyl Ethyl Keytone | *Most | * | * Most | X (dep. on |
| | likely X | | likely X | the grade) |
| Cyclohexanone | X | ◯ | * Most | * |
| | | | likely X | |
| Ethyl Acetate | X | ◯ | * Most | X |
| | | | likely X | |
| Dichloromethane | ◯ | ◯ | * Most | ◯ |
| | | | likely X | |
| Dimethylformamide | ◯ | ◯ | * most | ◯ |
| | | | likely ◯ | |
| Dimethylacetamide | ◯ | ◯ | * Most | ◯ |
| | | | likely X | |
| Tetrahydrofuran | X | X | * Most | X |
| | | | likely ◯ | |
| N-methylpyrrolidone | ◯ | ◯ | * Most | ◯ |
| | | | likely X | |

In some implementations, SMA may be used as a coating in a catheter system for outside diameter/inside diameter (OD/ID) lubricity by the hydrophilic block of an SMA including polyvinylpyrrolidone (PVP), polyethylene glycol (PEG), poly(acrylamide) (PAAm), poly(n-isopropylacrylamide) (NIPAAM), carboxymethyl cellulose (CMC), and other polymers.

In some implementations, SMA may be used as a coating in a catheter system as an OD/ID low frictional surface. For example, in such applications, the SMA may be within the Fluorinated block including, but not limited to, hexafluoropolypropylene (HFP), vinylidene fluoride (VDF), and other fluoropolymers.

In some implementations, SMA may be used as a coating in a polymeric implant or a catheter system for OD/ID thromboresistance. For example, in such applications, the SMA may be within the functional block including, but not limited to, PEG, polycarbonate (PC), polyvinylidene difluoride (PDVF), terpolymer of tetrafluoroethylene, hexafluoropropylene, and vinylidene fluoride (THV), and other polymers. In some implementations, the OD and the ID can be coated asymmetrically to have two different SMA listed above. For example, PEG may be used on the OD and PVDF on the ID.

In some implementations, the SMA architecture may be modified using unsaturated allyl or acrylate or —SH groups on the SMA. Following bloom to the surface, these groups can be used to crosslink with a surface coating, including a hydrophilic coating for a catheter. In some implementations, a tri-block architecture (flanking blocks being immiscible with bulk polymer), instead of a di-block architecture, increases thermodynamic driving force to the surface through various block size ratios and the molecular weights of the flanking and bulk polymer blocks. In some implementations, tri-block architecture (flanking blocks being miscible with bulk polymer), instead of di-block architecture, increases the stability on the surface of the polymer through various block size ratios and the molecular weights of the flanking and bulk polymer.

In some implementations, the SMA architecture may be modified to include a thromboresistant head group, for example fluorine or PEG, so that following bloom to the surface, thromboembolic resistance is conferred to the surface of the implant that is in contact with blood.

The coating material(s) may be applied to the implant surface according to a number of processes, depending on the composition selected. These processes may include but are not limited to: plasma vapor deposition; glow discharge deposition; chemical vapor deposition; low pressure chemical vapor deposition; physical vapor deposition (liquid or solid source); plasma-enhanced chemical vapor deposition; plasma-assisted chemical vapor deposition; thermal cracking (e.g., with fluoropolymers such as Parylene), spray coating; dip coating; spin coating; magnetron sputtering; sputter deposition; ion plating; powder coating; thermal spray coating; silanization; and/or layer-by-layer polymerization. Some processes (e.g., silanization or layer-by-layer polymerization) may be particularly useful for forming thin coatings. The application processes may be broadly categorized as vapor deposition processes or solution-based processes. In some implementations, the vapor deposition processes may proceed according to equilibrium reactions or non-equilibrium reactions and may use stable precursors or easily vaporized active precursors. Vapor deposition processes may be particularly well-suited for fabrication of conformal coatings, in which a particular composition is applied only selectively to distinct regions of the device, especially where complex patterns or geometries are involved. Vapor depositions can be performed relatively quickly and can easily produce thin high-integrity coatings (e.g., less than 20 nm, 20-50 nm, 50-75 nm, 75-100 nm, 100-150 nm, 150-300 nm, 300-500 nm, greater than 500 nm, or a thickness from any range there between). Solution-based processes may result in highly reliable molecular architectures and can be readily amenable to sterilization without altering the molecular architecture and/or biological activity. Many of the materials may be cured subsequent to application by heat melting and/or by cross-linking.

In some implementations, the implant may be primed prior to application of a coating. Priming the implant can facilitate attachment of the coating to the implant (e.g., to the struts or wires of the implant). Priming of the surface may be by mechanical means, such as media blasting, sanding, scribing, etc. Mechanical priming may increase the surface area of the implant. Increasing the surface area may promote adhesion of coating molecules and/or cells (e.g., endothelial cells). In some implementations, electropolishing of the implant can be deoptimized to attain at least some surface roughness on the implant. Priming of the surface may be by chemical means such as etching (e.g., plasma etching), or other surface functionalization, such as bombardments with hydrogen or nitrogen ions to activate molecular bonding sites. Priming of the surface may be by pre-coatings with substrates that help with adherence of the final polymer coating, such as vapor-deposition of substrates (e.g., parylene, silane, etc.), sputtered coatings, and/or electroplated coatings (e.g., with platinum, gold, aluminum oxide). In some implementations, multiple layers of a coating or multiple coatings may be applied to the implant. Priming of the implant can include growing extra material (e.g., nitinol) on the surface. Priming can be performed by a sacrificial particle technique, wherein particles are attached to the surface, extra material of the implant is grown over such particles, and then the implant is heat cycled to cause the particle, the extra material grown over it, and at least some material that became attached to such extra material to be removed from the implant surface to create a microporous surface. Priming may be performed on the underlying implant and/or on one or more coatings of the implant. In some implementations, the priming layer may be reacted off of portions of the implant, for example using thermal treatment, photochemical treatment, sonic treatment, and/or treatment with an electromagnetic field.

In some implementations, the surface hydrophobicity of the implant can be increased to improve primer and/or coating adhesion, particularly when such primer(s) and/or coating(s) are applied directly to a nitinol implant surface. Nitinol implants that are electropolished, for example, to reduce the risk of corrosion thereof, can resultingly have a thin surface layer of titanium oxide. This layer of titanium oxide can change its surface wettability in response to UV light. For example, the air-water contact angle of electropolished nitinol has been reported as generally ranging from 45 to 95 degrees (e.g., from very hydrophilic to very hydrophobic). Primers such as PEI or PAV can bind to surfaces using the hydrophobic effect, thus such a variation in the nitinol surface free energy can have an impact on primer and coating adhesion.

The method of cleaning an implant can increase the surface hydrophobicity of the implant. For example, bleach can be used to clean an implant. A hydrocarbon can be applied to the implant to increase the surface hydrophobicity thereof. For example, an alkane (hexane, heptane, octane, or higher alkane) or a solvent with a high boiling point that is largely immiscible with water, such as cyclohexanone, can be applied to the implant to increase its hydrophobicity. A fluorocarbon can be applied to the implant to increase the surface hydrophobicity thereof. For example, perfluorooctane (CF₃(CF₂)₆CF₃), Fluorinert FC-40 or FC-70, or 1,1,2,2,9,9,10,10-Octafluoro [2,2]paracyclophane can be applied to the implant to increase its hydrophobicity. An implant surface can be modified using silanes, such as an aminosilane such as (3-Aminopropyl) triethoxysilane or such as carboxy-silane triol, to increase the hydrophobicity thereof. For example, an implant surface can be modified using carboxyethylsilanetriol, disodium salt; or N-(trimethoxysilylpropyl)ethylenediamine, triacetic acid, trisodium salt (available from Gelest (Mitsubishi Chemical)) to increase the surface hydrophobicity thereof. The carboxy functional groups provided by these silanes may bond covalently with poly(allyl amine) hydrochloride. The implant surface can be modified using flurosilanes such as (tridecafluoro-1,1,2,2-tetrahydrooctyl) silane (available from Gelest (Mitsubishi Chemical)) or 1H,1H,2H,2H-Perfluorooctyltriethoxysilane (available from Millipore-Sigma) or Trifluoropropyltrimethoxysilane (available from Shin-Etsu) to increase the hydrophobicity thereof. In some implementations, the silane and primer may be combined into a single step, for example, with dimethoxysilylmethylpropyl modified (polyethylenimine), or trimethoxysilylpropyl modified (polyethylenimine) (available from Gelest).

Alternatively or in addition to increasing the surface hydrophobicity of a nitinol implant, there are various other ways to improve the bonding and/or durability of bond of a coating as described herein (e.g., a heparin coating) with a nitinol implant surface. A nitinol implant surface can be modified by physical vapor deposition of a tantalum layer, such as offered by Denton Vacuum (Mooresetown, NJ), to increase the hydrophobicity and/or improve the bonding thereof. A nitinol implant surface can be coated with aluminum oxide (Al₂O₃) by vapor deposition to increase the hydrophobicity and/or improve the bonding thereof. A nitinol implant surface can be sputter coated with carbon or platinum to increase the hydrophobicity and/or improve the bonding thereof. A nitinol implant surface can be roughened to increase the hydrophobicity and/or improve the bonding thereof. A nitinol implant surface can be roughened by mechanical abrasion, such as microblasting, to increase the hydrophobicity and/or improve the bonding thereof. A nitinol implant surface can be roughened by wet chemical etching, for example, with chemical etchants H₂SO₄/H₂O₂, HCl/H₂SO₄, and NH₄OH/H₂O₂, to increase the hydrophobicity and/or improve the bonding thereof. A nitinol implant surface can be coated with polydimethylsiloxane (PDMS), for example, with molecular weights as low as about 100 and as high as about 100,000, to increase the hydrophobicity and/or improve the bonding thereof. In some implementations, a nitinol implant surface can be roughened by laser irradiation followed by coating with a solution of PDMS, and then curing the PDMS, to increase the hydrophobicity and/or improve the bonding thereof. A nitinol implant surface can be roughened by magnetic field-assisted electrical discharge machining or by mangetoelectropolishing to increase the hydrophobicity and/or improve the bonding thereof. A nitinol implant surface may be made hydrophobic or more hydrophobic by the addition of a doping element into the nitinol allow, such as NiTiTa or NiTiCr. A nitinol implant surface may be made hydrophobic or more hydrophobic by plasma treatment, for example using a fluorocarbon or HF gas. A nitinol implant surface may be made hydrophobic or more hydrophobic by deposition of a coating from P2i. In some implementations, a titanium oxide layer on the surface of a nitinol implant can be made hydrophobic or more hydrophobic by exposure to infrared light. A nitinol implant surface can be coated with parylene, including parylene-C, parylene-N, parylene-F, parylene-D, parylene-HT, and parylene-AF4, to increase the hydrophobicity and/or improve the bonding thereof.

Additionally or alternatively, the implant can include a complete or partial luminal layer of endothelial cells or be seeded with endothelial cells prior to implantation.

In some implementations, the coating can be treated to increase an adhesion of the coating to the implant. For example, all solvent or substantially all solvent may be removed from the coating to allow the polymer chains to rearrange and/or compact. Exemplary implementations to improve coating adhesion include, but are not limited to: chemical etching, particulate etching, saturating the environment with evaporated solvent, heat treatment, and/or post coating solvent dip or spray, each of which will now be described in turn.

Chemical etching (e.g., with HF, HF+HNO₃, etc.) may provide a textured surface that may allow for the coating to have more surface area for adhesion. This method can produce a wide range of surface roughness. Particulate etching (e.g., with plastic parts or baking soda) can also roughen the surface of the device. This can act the same way as the chemical etching but will leave larger defects because the etching particles are larger.

Further, saturating the environment with evaporated solvent that is the same solvent used in the solution to coat the implant allows for the coating to evenly spread over the implant surface without allowing the coating to dry before a uniform or substantially uniform coating is produced.

Heat treatment of the coated implant after the coating is completed can increase solvent removal from the coating and smooth the surface of the coating. The heat treatment may occur at a temperature of 30° C. to 80° C. Alternatively or additionally, heat treatment at a high temperature can remove solvent as well as allow the polymer chains to arrange in a tightly packed formation, producing an even thinner and smoother coating. For example, the heat treatment may occur at a temperature of 81° C. to 250° C.

A post coating solvent dip or spray may also or alternatively smooth the surface of the coating and/or reduce a thickness of the coating. In such implementations, a suitable solvent is one that was used in the original coating solution and/or one that dissolves the polymer. The dip or spray step may occur over a short interval of time or reach equilibrium before the original coating is fully removed.

In some implementations, plasma cleaning prior to coating leaves a slight charge on the surface of the stent implant and can allow for a smoother coating.

The coating is preferably thin to reduce the risk of debris creating dangerous emboli, especially in neurovascular applications. For the same reason, the coating is preferably durable and not prone to produce debris upon friction created when the implant is expanded (e.g., when the struts may rub against each other or parts of its delivery device). In some implementations, the coating is no greater than about 300 nm thick. Coating materials that are mechanically robust and do not flake or fracture after coating may be particularly suitable for thicker coatings (e.g., 300 nm thick coatings). In some implementations, the coating is no greater than about 3 nm, 5 nm, 8 nm, 10 nm, 15 nm, 20 nm, 25 nm, 30 nm, 40 nm, 50 nm, 60 nm, 75 nm, 100 nm, 150 nm, 200 nm, or 300 nm thick. In some implementations, coatings may be greater than 300 nm thick or less than 3 nm (e.g., Angstrom levels). Thinner coatings (e.g., 75 nm thick or less) may provide robust performance in endothelialization and/or anti-thrombogenicity while minimizing the coating's mechanical contribution to the flow characteristics through the central lumen of the implant. Thinner coatings may be less likely to produce particulate debris of larger sizes that could pose risks of embolization, such as stroke. In preferred implementations, the coating may be between about 4-15 nm thick, 5-20 nm thick, 5-30 nm thick, 25-50 nm thick, 30-50 nm thick, 30-40 nm thick, 40-50 nm thick, 35-40 nm thick, 40-60 nm thick, 50-60 nm thick, less than 25 nm thick, less than 15 nm thick, greater than 4 nm thick, or greater than 60 nm thick. Coatings within optimal ranges may provide sufficient surface coverage and reduced thrombogenicity while minimizing potential toxicity concerns. For example, in some implementations, even if all the coating were stripped from the device, the amount of coating material in the thin coating would be below a toxicity threshold. The coating coverage and thickness may be determined by scanning electron microscopy (SEM). In some implementations, 100% surface coverage is achieved. In some implementations, less than 100% surface coverage is achieved (e.g., 25%, 50%, 75%, 80%, 90%, less than 25%, between 90-100%, or any range there between). In some implementations, the device may not need 100% surface coverage to achieve sufficient anti-thrombogenic properties. Durability may be evaluated by performing SEM before and after simulated fatigue. Also, in preferred implementations, the coated implant satisfies the USP 788 standard. That is, it produces no more than 600 particles that are 25 μm or larger and no more than 6000 particles that are between 10 μm and 25 μm. Furthermore, the implant preferably does not generate particulate less than about 2 μm in size.

The coating or coatings may be applied differentially to different regions of the implant. In some implementations, the inner diameter of the implant lumen is applied with a coating which optimizes thromboresistance while the outer diameter of the implant is applied with a coating that optimizes endothelialization or vice-versa. Alternatively, the outer diameter of the implant may be uncoated (e.g., only an inner diameter of the implant is coated), to reduce the risk of embolic debris as the implant is advanced through the delivery device and deployed in a blood vessel. In some implementations, the coating is optimized to promote endothelialization toward the middle of the implant (e.g., along a portion configured to be positioned proximate an aneurysm neck) and to reduce thrombosis towards the proximal and distal ends of the implant. Promoting endothelialization near the aneurysm neck may facilitate growth of an intimal layer which occludes the aneurysm from the blood vessel. Various combinations of the aforementioned spatial distribution may also be applied. One specific implementation may include anti-CD 34 endothelial progenitor cell (EPC) capture coating in the middle segment of the implant apposed against the aneurysm sac opening while the proximal and distal ends are coated with PVDF-HFP. Another implementation may include a spatially distributed pattern of PVDF-HFP and EPC capture coating intermixed on the inner diameter of the implant. The distribution pattern metric may be quantified by spatial periodicity of the EPC domains and size of the EPC domains, combination of these two metrics will determine the overall area fraction of EPC domains and PVDF-HFP domains. One special case will be 100% coverage with EPC capture coating. The spatial distribution of multifunctional coating patterns will provide thromboembolic protection at different timescales acute (t=0−1 d), sub-acute (t=1−30 d), and long term (>30 d). Mechanistically, the EPC/PVDF-HFP patterned coating will be thromboembolism-resistant by modulating blood protein and platelets on the surface and at the near-surface region. An additional biological outcome will be faster isolation and sealing of the aneurysm sac from the parent vessel. Furthermore, such a variation in properties along the length of the implant may be attained as a gradient in properties rather than as distinct regions with distinct properties. The difference in properties may be accomplished by altering the composition of the coating and/or by differentially processing the coating during its application. The composition of the coating at any point may comprise one or more of the materials discussed above. In some implementations, such conformal coating strategies may be used to promote endothelialization of the implant along the aneurysm neck, such that the aneurysm eventually becomes sealed off from the native lumen of the blood vessel.

At least some of the surface modifications described herein can be categorized as true coatings (25-1000 nm, or up to 5000 nm). Coatings may be deposited macroscopically, for examples PVDF-HFP, THV, PC-PBMA, PEG-PBMA. Alternatively coatings may be applied using a surface grafting approach (thickness in the range of 2-25 nm or up to 100 nm; molecular level surface reaction; examples Fluorination, PC-grafting, PEG grafting, or heparin grafting).

Different regions of the coated implant may achieve thromboresistance through different mechanisms of action. For example, the coating may act to prevent platelets from sticking-effective in relatively high shear, high velocity regions and not so much in stasis, low flow regions where thrombin-fibrin are more likely to initiate and grow thrombus. Therefore it may be important to minimize potential stasis points. This may be accomplished by minimizing total leading edge area and rounding the leading edge of each strut or strut portion that obstructs blood flow and optionally also rounding the trailing edges of struts that face the downstream direction. Also when the concave side of an apex faces upstream, the apex can provide a potential stasis point. Presetting the ‘downstream’ pointing apexes so they are biased radially outwardly allows them to embed a little more deeply into the adjacent vessel wall and lower the profile of the apex to reduce interference with blood flow. The upstream pointing apexes may be biased radially outwardly as well.

The physical design of the implant may impact its biocompatibility, particularly by the manner in which it alters natural blood flow. Platelet activation may be reduced by decreasing the stress platelets experience as blood flows across the implant. Both the amount of device material the blood encounters as it flows (i.e. the fraction of the blood vessel cross section occupied by the device) as well as the angle at which the device interfaces the blood flow (the take-off angle) can influence the stress experienced by platelets and their resulting activation.

The implant may be a permanent or temporary intravascular scaffold, such as a deployable vascular stent or a temporary scaffold. In some implementations, the implant may be an aneurysm treatment device. In such implementations, the implant provides mechanical support for the coils or other embolic implant, to prevent them from falling into the blood stream and enables a higher packing density of coils. In some implementations, the implant may temporarily retain the coils or implants within the aneurysm. Once the packing density of the coils is high enough, the coils may exert sufficient pressure on each other to retain the coils within the aneurysm and prevent them from falling through the aneurysm neck and into the blood stream. In some implementations, the implant may remain implanted within the blood vessel and may facilitate retention of the coils within the aneurysm. The implant may extend beyond the edge of the aneurysm neck by at least about 3 mm or 4 mm or more in both proximal and distal directions to mechanically support the borders.

Prior to expansion, the implants described herein may be sized to be received within a tubular delivery sheath/catheter with an internal diameter of about 0.41 mm to about 0.54 mm (e.g., the outer diameter of the implant may be about 0.40 mm to about 0.48 mm collapsed). In various implementations, a central portion (or portion of the implant that interfaces an aneurysm) has gaps between struts (e.g., the largest dimension of the gap) that are less than about 0.125 mm, less than about 0.150 mm inches, less than about 0.175 mm, less than about 0.225 mm, less than about 0.250 mm, less than about 0.275 mm, less than about 0.300 mm, less than about 0.325 mm, less than about 0.350 mm, less than about 0.375 mm, less than about 0.400 mm, or more than about 0.400 mm. In some implementations, the gaps are preferably no more than about 0.200 mm to prevent escape of the coils and to promote a high coil packing density. In some implementations, the gaps between struts of the implants described herein may be as small as practical but large enough to allow a micro-catheter to pass therethrough (0.500 mm to 1.1 mm). In some implementations, the gaps between struts near proximal and/or distal ends of the implants described herein may be larger than gaps between struts positioned adjacent the aneurysm neck (e.g., near the middle of the implant). Areas with larger gap dimensions may create localized areas of low-density compared to areas with smaller gap dimensions. In some implementations, interstitial gaps in areas of low-density may have about 105%, 110%, 115%, 120%, 125%, 130%, 135%, 140%, 150%, 175%, 200%, 250%, 300%, 400%, 500%, 600% 700%, 800%, 900%, 1000%, 2000%, 5000%, between 100% and 105%, more than 5000%, or any percentage in a range there between, larger areas or dimensions (e.g., diameter) than interstitial gaps in areas of high-density.

Although the intraluminal implants, devices, systems and methods disclosed herein are described in a particular manner which can provide certain advantages, such description is not intended to be limiting. The intraluminal implants described herein can be implanted in various vessels and/or passageways of a patient, including vessels (e.g., veins, arteries) of the patient's vascular system, the patient's lymphatic system, the patient's reproductive system, etc.

Any and/or all of the implementations and/or features of the intraluminal implants, devices, systems and methods described and/or illustrated herein can be applied to and/or utilize the various devices, systems and methods described and/or illustrated in U.S. Provisional Patent Application No. 63/281,923, filed Nov. 22, 2021, titled “NEUROVASCULAR DEVICES HAVING THREE DIMENSIONAL CONFIGURATIONS AND SURFACE CHEMISTRIES FOR ENHANCED THROMBORESISTANCE AND/OR ENDOTHELIALIZATION,” and incorporated by reference herein in its entirety. For example, any and/or all of the implementations and/or features of the intraluminal implants, devices, systems and method described and/or illustrated herein, such as a thromboresistant intraluminal implant and associated delivery device, can be applied in U.S. Provisional Patent Application No. 63/281,923.

### Intraluminal Implant

FIG. 1A illustrates a simplified representation of the anatomy of a subject 1 with an implant delivery catheter 1100 used to establish a percutaneous path through the subject's vasculature to a neurovascular site of the subject for delivery of an implant 100 via a delivery wire 600. FIG. 1B illustrates a simplified representation of the basilar and non-basilar anatomy of the subject 1. The implants, devices, and systems described herein can be configured to access and deploy within the anatomy shown in FIG. 1B or elsewhere in the subject's body as described herein.

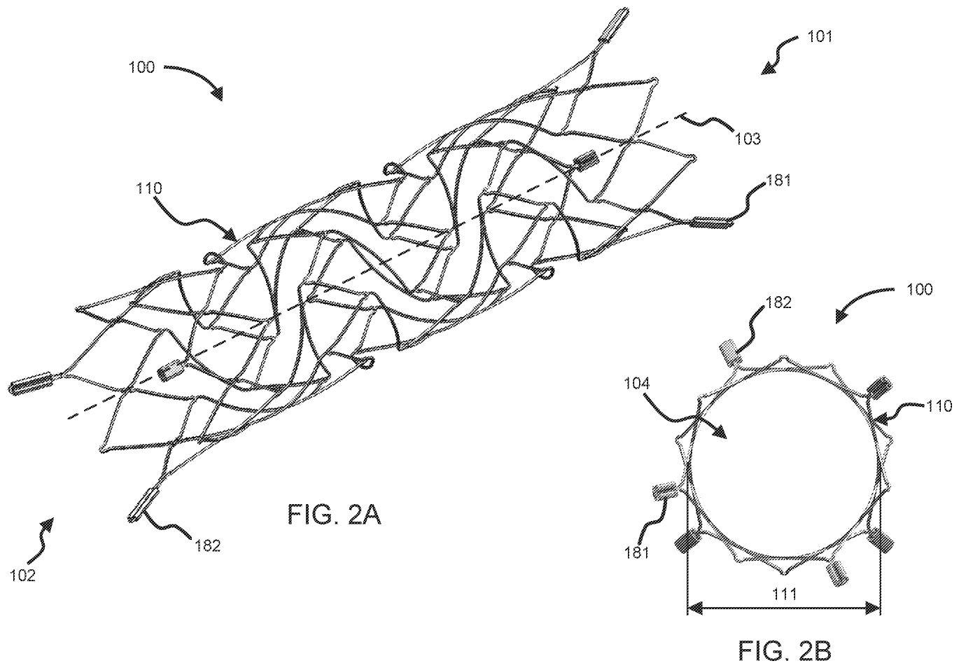

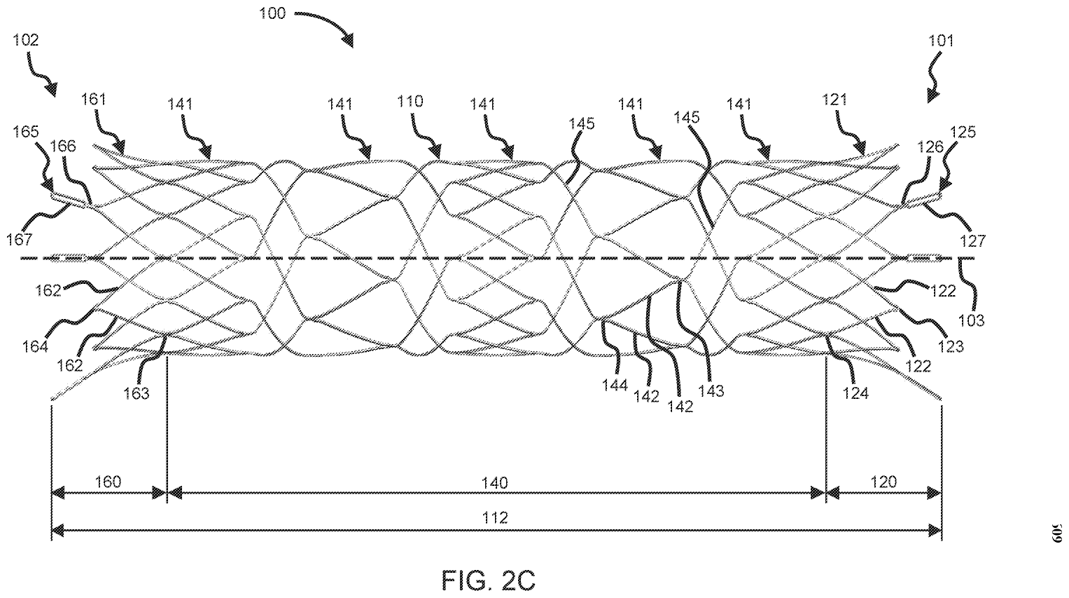

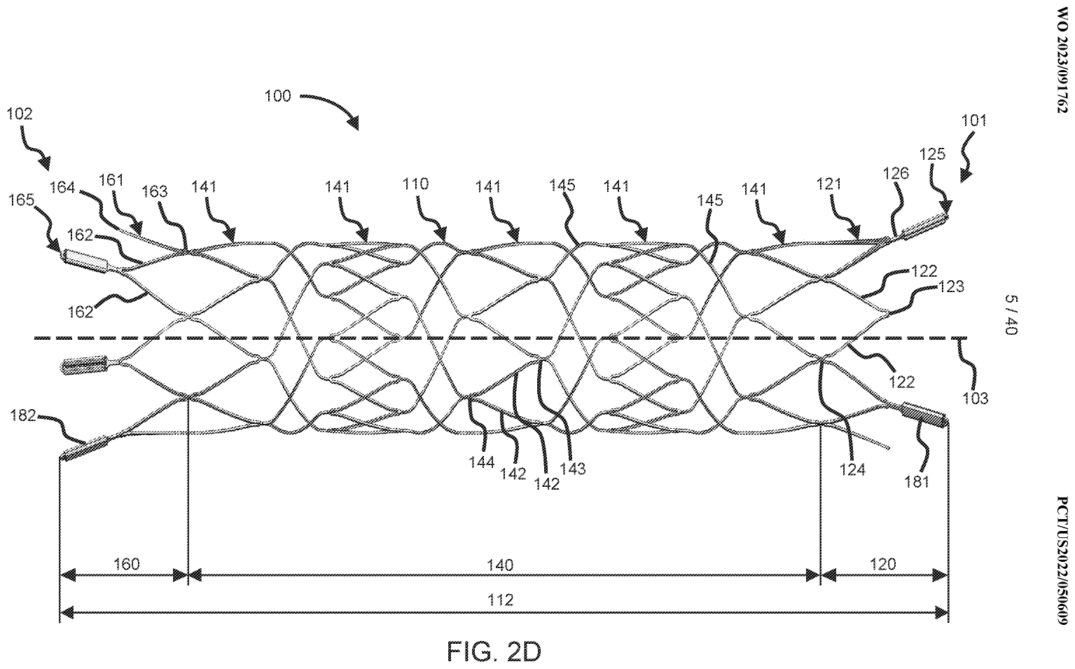

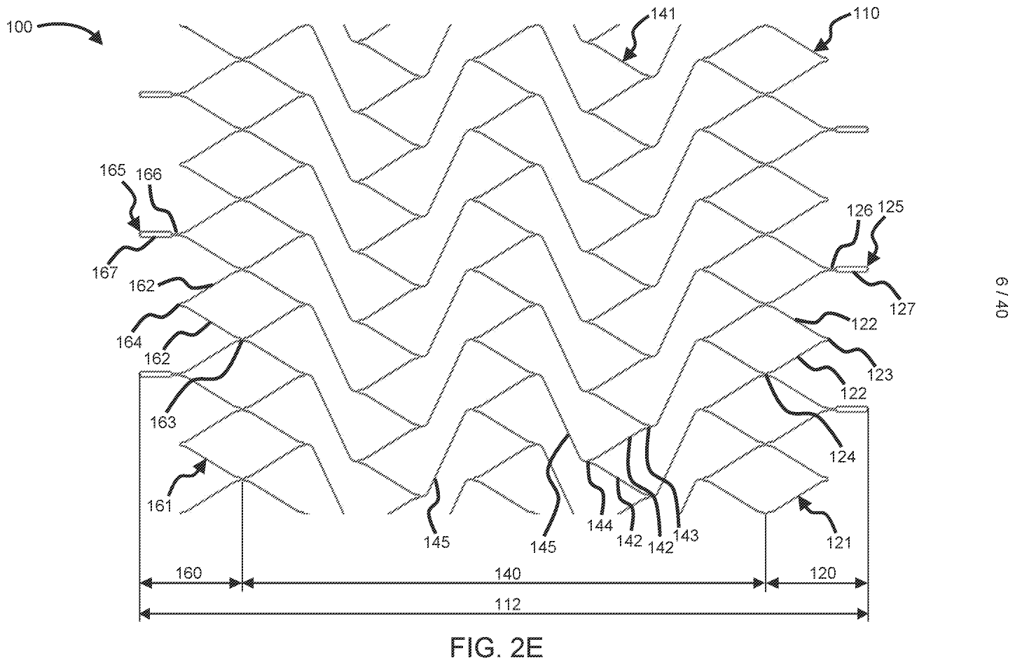

FIGS. 2A-2E illustrate an implementation of an intraluminal implant 100. FIG. 2A is a perspective view, FIG. 2B is an end view, FIGS. 2C-2D are side views, and FIG. 2E is a flattened pattern view of the implant 100. The implant 100 can be cut (e.g., laser cut) from tubing to form the generally tubular frame 110 shown. The tubing used to form the implant 100 can be a shape memory and/or superelastic material, such as nitinol. Furthermore, the tubing cut to form the tubular frame 110 can be expanded tubing that is about the same diameter as the final implant diameter (e.g. the unconstrained/expanded diameter), which can be an advantageous method of manufacturing over shape setting to the final diameter. In some implementations, the implant can be cut from tubing that is later shape set to the final diameter, or the implant can be made of wire shape set to the configurations shown and described herein. The tubular frame 110 of the implant 100 can generally be cut, shape set, media blasted (e.g., to remove any layer of carbon resulting from shape setting), and electropolished. Electropolishing can advantageously round off edges of the tubular frame 110. The implant 100 can be self-expanding and have a collapsed or crimped configuration for delivery and an expanded configuration for implantation.

As shown in at least FIG. 2A, the implant 100 can generally have a proximal end 101 and a distal end 102 with the tubular frame 110 defining a lumen 104 having a longitudinal axis 103. Further as shown, the implant 100 can include one or more radiopaque markers. Such radiopaque markers can be disposed at or adjacent the proximal end 101 and/or the distal end 102. For example, and as shown in FIG. 2A, the implant 100 can include one or more proximal radiopaque markers 181 at the proximal end 101, and one or more distal radiopaque markers 182 at the distal end 102. In some implementations, the implant 100 can include at least one proximal radiopaque marker 181, such as one, two, three, four, five, or more proximal radiopaque markers 181. In some implementations, the implant 100 can include at least one distal radiopaque marker 182, such as one, two, three, four, five, or more distal radiopaque markers 182. Such radiopaque markers can be connected (e.g., crimped) to the implant 100 (e.g., connected to the tubular frame 110 of the implant 100) or be an integral part of the implant 100. The radiopaque markers 181, 182 can aid in visualization of the implant 100 during delivery and implantation. In some implementations, the radiopaque markers 181, 182 are sized and configured to be at or just above the threshold of visibility when imaged during delivery of the implant 100. Such sizing and configuration of the radiopaque markers 181, 182 can help ensure the radiopaque markers themselves do not produce thrombi after implantation and provide for a thromboresistant implant 100.

As further shown in at least FIG. 2A, portions or ends of the implant 100 can be flared radially outward. For example, a portion of the implant 100 adjacent the proximal end 101 can be flared radially outward in the proximal direction, and/or a portion of the implant 100 adjacent the distal end 102 can be flared radially outward in the distal direction. Such flaring can advantageously: ensure apposition between the proximal and distal ends of the implant 100 (and any radiopaque markers of the implant 100, such as radiopaque markers 181, 182) and a wall of a vessel in which the implant 100 is implanted, particularly at or near turns or curves in the vessel; ensure the lowest profile possible at the proximal and distal ends of the implant 100 when implanted in a vessel, which can minimize or eliminate any effects of the implant 100 on the flow of bodily fluid (e.g., blood) through the vessel at the site of implantation; aid in securement of the implant 100 at the desired position during delivery and when implanted in a vessel; and/or aid in preventing migration of the implant 100 from the desired position when implanted in a vessel. In some embodiments, the implant 100 does not have flared proximal and/or distal portions or ends.

The implant 100 (e.g., the tubular frame 110 of the implant 100) can have a thickness (e.g., wall thickness) of between about 10 microns (μm) to about 100 μm, about 20 μm to about 90 μm, about 25 μm to about 80 μm, about 30 μm to about 70 μm, about 35 μm to about 60 μm, about 40 μm to about 55 μm, about 40 μm to about 50 μm, about 40 μm, about 41 μm, about 42 μm, about 43 μm, about 44 μm, about 45 μm, about 46 μm, about 47 μm, about 48 μm, about 49 μm, less than about 60 μm, less than about 50 μm, or more than about 25 μm. Such thin wall thickness can advantageously minimize or eliminate any effects of the implant 100 on the flow of bodily fluid (e.g., blood) through the vessel at the site of implantation. The width of the struts of the implant 100, such as the plurality of ring struts 122, 142, 162, the plurality of linking struts 145, the one or more proximally extending struts 125, and/or the one or more distally extending struts 165, can be about the same as their wall thickness (e.g., the wall thickness of the tubular frame 110). In some implementations, the width of the plurality of linking struts 145 is less than the width of the plurality of ring struts 122, 142, 162. Such a configuration can make the implant 100 more flexible, kink resistant, and/or conformal to an adjacent vessel wall. In some implementations, the widths of the struts of the implant 100 are about the same as one another. In some implementations, at least some of the widths of the struts of the implant 100 are different from one another.

The implant 100 (e.g., the tubular frame 110 of the implant 100) can have a diameter 111 of between about 1 mm to about 6 mm, about 1.5 mm to about 5.5 mm, about 2 mm to about 5 mm, about 2.5 mm to about 4.5 mm, about 3 mm to about 4 mm, about 3 mm, about 4 mm, less than about 5 mm, or more than about 2 mm. Such diameter can be measured along a central portion of the implant 100 (e.g., not including the flared distal and proximal ends/portions if included) when in its expanded/unconstrained state.

The implant 100 (e.g., the tubular frame 110 of the implant 100) can have a length 112 of between about 5 mm to about 70 mm, about 8 mm to about 65 mm, about 10 mm to about 60 mm, about 12 mm to about 55 mm, about 15 mm to about 50 mm, about 15 mm, about 16 mm, about 20 mm, about 23 mm, about 30 mm, about 40 mm, about 50 mm, less than about 50 mm, less than about 25 mm, or more than about 12 mm. Such length can be measured when the implant 100 is in its expanded/unconstrained state.

FIG. 2C shows a side view of the implant 100 without radiopaque markers (e.g., showing only the tubular frame 110), while FIG. 2D shows a side view of the implant 100 with radiopaque markers 181, 182. Generally, the implant 100 can include the tubular frame 110 and the radiopaque markers 181, 182. As shown in these side views, the implant 100 (e.g., the tubular frame 110) can generally include a plurality of longitudinally spaced apart rings that extend along a circumference of the tubular frame 110. The plurality of rings can generally be connected to one another by a plurality of linking struts that extend at least partially along the circumference of the tubular frame 110.

With continued reference to FIGS. 2C-2D, the implant 100 (e.g., the tubular frame 110) can include a proximal portion 120, a distal portion 160, and a central portion 140 between the proximal portion 120 and the distal portion 160. The proximal portion 120 can be located adjacent the proximal end 101, and the distal portion 160 can be located adjacent the distal end 102. The proximal portion 120 can include a ring 121 that extends along the circumference of the tubular frame 110. The ring 121 can include a plurality of ring struts 122, with adjacent pairs of ring struts joining at a plurality of proximal apexes 123 and a plurality of distal apexes 124 to form a chevron pattern as shown. Similarly, the distal portion 160 can include a ring 161 that extends along the circumference of the tubular frame 110. The ring 161 can include a plurality of ring struts 162, with adjacent pairs of ring struts joining at a plurality of proximal apexes 163 and a plurality of distal apexes 164 to form a chevron pattern as shown. In implementations of the implant 100 that include flared proximal and/or distal ends/portions, such flaring can begin at the proximal and/or distal portions 120, 160, respectively (e.g., where the proximal and/or distal portions 120, 160 connect to the central portion 140). The central portion 140 can include a plurality of longitudinally spaced apart rings 141 that extend along the circumference of the tubular frame 110. Each ring of the plurality of rings 141 can include a plurality of ring struts 142, with adjacent pairs of ring struts joining at a plurality of proximal apexes 143 and a plurality of distal apexes 144 to form a chevron pattern as shown. While FIGS. 2C-2D show an implant 100 with a central portion 140 having 5 rings 141, the implant 100 can include less than 5 rings 141, 5 rings 141, or more than 5 rings 141.

The central portion 140 can also include a plurality of linking struts 145 that extend at least partially along the circumference of the tubular frame 110. Each linking strut of the plurality of linking struts 145 can connect a distal apex of one ring of the plurality of rings 141 to a proximal apex of an adjacent ring of the plurality of rings 141 as shown. Also as shown, each linking strut of the plurality of linking struts 145 can connect each one of the plurality of distal apexes 144 of one ring of the plurality of rings 141 of the central portion 140 to each one of the plurality of proximal apexes 143 of an adjacent ring of the plurality of rings 141 of the central portion 140 except for at each one of a plurality of distal apexes of a distal most ring of the central portion 140 and except for at each one of a plurality of proximal apexes of a proximal most ring of the central portion 140 such that the central portion 140 does not comprise any free apexes (e.g., no unconnected apexes). In other words, the implant 100 can be configured to have no untethered apexes between its proximal end 101 and its distal end 102, although it may have free apexes at its proximal end 101 and its distal end 102 as shown. Such configuration can advantageously aid in repositioning of the implant 100 if needed during delivery since there are no apexes to catch on a distal edge/end of a delivery catheter and/or on tissue. Furthermore, such configuration can advantageously aid in repositioning or removal of the implant after implantation of the implant 100.

As further shown in at least FIGS. 2C-2D, each distal apex of the plurality of distal apexes of the distal most ring of the central portion 140 can connect to a respective proximal apex of the plurality of proximal apexes 163 of the ring 161 of the distal portion 160. Similarly, each proximal apex of the plurality of proximal apexes of the proximal most ring of the central portion 140 can connect to a respective distal apex of the plurality of distal apexes 124 of the ring 121 of the proximal portion 120. Furthermore, as shown, each distal apex of the plurality of distal apexes 144 of a ring of the plurality of rings 141 of the central portion 140 can be rotationally offset from each proximal apex of the plurality of proximal apexes 143 of an adjacent ring of the plurality of rings 141 of the central portion 140. In such a configuration, at least a portion of each linking strut of the plurality of linking struts 145 connecting such rotationally offset distal apexes 144 and proximal apexes 143 can extend along a helical path at least partially around the circumference of the tubular frame 110. Such helical path can extend in a first helical direction between a set of adjacent rings 141 of the central portion 140 and extend in a second helical direction that is generally opposite the first helical direction between the next set of adjacent rings 141 of the central portion 140 as shown. In other words, a row of linking struts 145 (e.g., joining a pair of adjacent rings 141) can extend at least partially around the circumference of the tubular frame 110 in one helical direction, and a next row of linking struts 145 (e.g., joining a next pair of adjacent rings 141) can extend at least partially around the circumference of the tubular frame 110 in an opposite helical direction. As shown, the plurality of linking struts 145 can be configured such that they do not overlap one another.

The implant 100 (e.g., the tubular frame 110 of the implant 100) can thus generally include rings, such as rings 141 that can have a chevron-like configuration, that alternate longitudinally with linking struts 145 as described above. Combined, such structure of the tubular frame 110 can provide for a highly conformable implant 100 to minimize implant-to-vessel malapposition. Also, such structure of the tubular frame 110 can allow for self-expansion of the implant 100 to a variety of different diameters and configurations of an adjacent vessel wall, rather than expanding to a substantially constant diameter throughout the length of the implant 100. For example, such configuration of the rings (such as rings 141, 121, and 161) can advantageously provide radial compliance of the implant 100 (e.g., the tubular frame 110 of the implant 100), such as to allow the implant 100 to expand and contract to conform to a vessel wall (e.g., an internal vessel wall). Furthermore, such helical winding of the plurality of linking struts 145 can advantageously provide longitudinal compliance of the implant 100 (e.g., the tubular frame 110 of the implant 100), such as to allow the implant 100 to expand along and conform to an outer part of a bend or turn of a vessel and contract along and conform to an inner part of a bend or turn of a vessel. Additionally, such alternating helical path of adjacent rows of linking struts 145, when present, can help resolve any torque or twisting of the implant 100.

In some implementations, the diameter of the implant 100 can be adjusted by increasing or decreasing the number of the plurality of ring struts 122, 142, and 162 that make up the rings 121, 141, and 161 of the proximal, central, and distal portions, respectively. With an increase or decrease in the number of the plurality of rings struts 142, the number of the plurality of linking struts 145 can increase or decrease in kind to ensure there are no unconnected distal apexes 144 and/or no unconnected proximal apexes 143. In some implementations, the length of the implant 100 can be adjusted by increasing or decreasing the number of the plurality of rings 141. With an increase or decrease in the number of the plurality of rings 141, the number of the plurality of linking struts 145 can also increase or decrease.

As shown in at least FIG. 2C and FIG. 2E, the implant 100 (e.g., the tubular frame 110 of implant 100) can include one or more generally proximally extending struts 125 and/or one or more generally distally extending struts 165. Such one or more generally proximally extending struts 125 can extend from a respective one or more proximal apex of the plurality of proximal apexes 123 of the ring 121 of the proximal portion 120. Similarly, such one or more generally distally extending struts 165 can extend from a respective one or more distal apex of the plurality of distal apexes 164 of the ring 161 of the distal portion 160. Each of the one or more generally proximally extending struts 125 and each of the one or more generally distally extending struts 165 can be configured to connect to a radiopaque marker, such as proximal radiopaque markers 181 and distal radiopaque markers 182, respectively. Each of the one or more generally proximally extending struts 125 can include a neck portion 126 and a connection portion 127, the connection portion 127 disposed proximal to the neck portion 126 and configured to connect to a proximal radiopaque marker 181. Similarly, each of the one or more generally distally extending struts 165 can include a neck portion 166 and a connection portion 167, the connection portion 167 disposed distal to the neck portion 166 and configured to connect to a distal radiopaque marker 182. For example, the connection portions 127, 167 can have an oblong shape with a through hole configured to receive a crimped on radiopaque marker. In some implementations, the implant 100 (e.g., the tubular frame 110 of the implant 100) can include at least one proximally extending strut 125, such as one, two, three, four, five, or more proximally extending struts 125. The number of proximally extending struts 125 can correspond to the number of proximal radiopaque markers 181. Similarly, in some implementations, the implant 100 (e.g., the tubular frame 110 of the implant 100) can include at least one distally extending strut 165, such as one, two, three, four, five, or more distally extending struts 165. The number of distally extending struts 165 can correspond to the number of distal radiopaque markers 182. The proximally extending struts 125 and/or the distally extending struts 165, when included, can extend in the proximal and distal directions, respectively, at an angle with the longitudinal axis 103, such as to continue as an extension of the outward radial flaring of the proximal and/or distal portions 120, 160. The proximally extending struts 125 and/or the distally extending struts 165, in combination with the proximal radiopaque markers 181 and/or the distal radiopaque markers 182, respectively, can be configured to releasably couple with a delivery wire for delivery of the implant 100 as will be described further below.

The implant 100 (e.g., the tubular frame 110 of the implant 100) can be configured to have a minimal abluminal surface area (e.g., outer surface area, which would be the surface area in contact with a vessel wall in which the implant 100 is implanted). For example, the implant 100 (e.g., the tubular frame 110 of the implant 100) can have an abluminal surface area of between about 3% to about 11%, about 4% to about 10%, about 5% to about 9%, about 4%, about 5%, about 5.5%, about 5.8%, about 6%, about 6.5%, about 7%, about 7.5%, about 8%, about 8.1%, about 8.5%, more than about 3%, or less than about 10%.

The implant 100 (e.g., the tubular frame 110 of the implant 100) can be configured to have a minimal end view surface area. In other words, the implant 100 can be configured to occupy a minimal fraction of the vessel cross section in which it is implanted. For example, the implant 100 (e.g., the tubular frame 110 of the implant 100) when viewed down its longitudinal axis 103 in an end view in its unconstrained/expanded state can occupy less than about 20%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, between about 3% to about 7%, between about 4% to 6%, about 4.5%, or about 5.9% of the cross sectional area defined by the outer diameter of the implant 100. In some implementations, the central portion 140 of the implant 100 (e.g., of the tubular frame 110) when viewed down its longitudinal axis 103 in an end view in its unconstrained/expanded state can occupy less than about 20%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, between about 3% to about 7%, between about 4% to 6%, about 4.5%, or about 5.9% of the cross sectional area defined by the outer diameter of the central portion 140 of the implant 100.

In some implementations, the implant 100 (e.g., the tubular frame 110 of the implant 100) is configured to have less malappositions between the implant 100 and an inner wall of a vessel in which it is deployed on an inside of a bend of the vessel than on an outside of the bend of the vessel. Such configuration can advantageously limit or eliminate potential areas of low flow or stagnant flow at an inside of a bend of the vessel and provide for a thromboresistant implant 100.

The implant 100 can have a mass of between about 0.50 mg and about 6.00 mg, between about 1.00 mg and about 4.00 mg, of about 2.00 mg, of about 2.10 mg, of about 2.20 mg, of about 2.30 mg, of about 2.40 mg, of about 2.50 mg, of about 2.60 mg, of about 2.70 mg, of about 2.80 mg, of about 2.90 mg, of about 3.00 mg, of at least about 0.50 mg, or no more than about 4.00 mg. For example, an implant 100 as described herein with a diameter of about 3.0 mm and a length of about 15 mm can have a mass of about 2.04 mg. As another example, an implant 100 as described herein with a diameter of about 3.0 mm and a length of about 20 mm can have a mass of about 2.09 mg. In another example, an implant 100 as described herein with a diameter of about 3.0 mm and a length of about 23 mm can have a mass of about 2.36 mg. As another example, an implant 100 as described herein with a diameter of about 4.0 mm and a length of about 20 mm can have a mass of about 2.50 mg. In another example, an implant 100 as described herein with a diameter of about 4.0 mm and a length of about 23 mm can have a mass of about 2.69 mg.

### Coating

The implant 100 can have a coating as described herein, such as a thromboresistant coating. For example, the implant 100, which includes the tubular frame 110 and any radiopaque markers when included such as radiopaque markers 181, 182, can have a heparin coating. The heparin coating can include a single layer or multiple layers. In some implementations, the coating of implant 100 can include a polyamine layer (e.g., a cationic polyamine layer) attached to the surface of the implant 100, and a heparin complex layer attached to the polyamine layer (e.g., attached via ionic interactions or covalent bonds). Furthermore, in some implementations, such a polyamine layer followed by a heparin complex layer can be repeatedly deposited so as to form multiple layers on the implant 100. For example, the implant 100 can have a polyamine layer, a heparin complex layer, a polyamine layer, a heparin complex layer, and so on repeatedly. Such repeated layering can produce an implant 100 having two alternating layers of polyamine and heparin, three alternating layers of polyamine and heparin, four alternating layers of polyamine and heparin, or more. The heparin coating of the implant 100, when included, can completely cover the implant 100 such that the implant 100 does not have any bare or uncoated portions. In some implementations, the heparin coating of the implant 100 is configured to be a permanent coating (e.g., a non-eluting coating). In some implementations, the heparin coating can be applied to a polymer layer (e.g., fluoropolymer) that has been applied to the surface of the implant 100. In some implementations, the heparin coating is applied directly to the surface of the implant 100, which can be a nitinol surface as described herein.

The heparin coating of the implant 100 can have a thickness of less than about 60 nm, less than about 50 nm, less than about 40 nm, less than about 30 nm, less than about 25 nm, less than about 20 nm, less than about 15 nm, less than about 14 nm, less than about 13 nm, less than about 12 nm, less than about 11 nm, less than about 10 nm, less than about 9 nm, less than about 8 nm, less than about 7 nm, less than about 6 nm, less than about 5 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, about 5 nm, about 4 nm, between about 3 nm to about 60 nm, between about 4 nm to about 30 nm, or between about 5 nm to about 20 nm. Such thickness of the heparin coating can be measured in the dry state (e.g., vacuum) using transmission electron microscope focused ion beam (TEM-FIB) imaging. Furthermore, such thickness of the heparin coating can be an average thickness of the thickness measured at various locations of the implant 100. The heparin coating of the implant 100 can have a uniform or substantially uniform thickness. For example, the thickness of the heparin coating of the implant 100 can be within three, two, or one standard deviations of the average thickness measured. A thin heparin coating can confer certain advantages. For example, if the entire coating were to delaminate and form a single embolic particle, it would be less than about 101 μm in diameter. If the entire coating delaminated and formed 10 μm in diameter particles, there would be only about 1000 particles, at least about six times below the limit from USP 788.

The heparin coating of the implant 100 can have a mass of less than about 1.50 μg, less than about 1.25 μg, less than about 1.00 μg, less than about 0.90 μg, less than about 0.80 μg, less than about 0.70 μg, less than about 0.60 μg, less than about 0.55 μg, less than about 0.50 μg, less than about 0.45 μg, less than about 0.40 μg, less than about 0.35 μg, less than about 0.30 μg, less than about 0.25 μg, about 0.75 μg, about 0.70 μg, about 0.65 μg, about 0.60 μg, about 0.55 μg, about 0.50 μg, about 0.45 μg, about 0.40 μg, about 0.35 μg, about 0.30 μg, between about 0.25 μg to about 0.75 μg, or between about 0.30 μg to about 0.60 μg.

The heparin coating of the implant 100 can have an activity (e.g., surface activity) of more than about 10 pmol AT/cm², more than about 15 pmol AT/cm², more than about 20 pmol AT/cm², more than about 25 pmol AT/cm², more than about 30 pmol AT/cm², more than about 35 pmol AT/cm², more than about 40 pmol AT/cm², more than about 45 pmol AT/cm², more than about 50 pmol AT/cm², more than about 55 pmol AT/cm², more than about 60 pmol AT/cm², more than about 65 pmol AT/cm², more than about 70 pmol AT/cm², about 20 pmol AT/cm², about 25 pmol AT/cm², about 30 pmol AT/cm², about 35 pmol AT/cm², about 40 pmol AT/cm², about 45 pmol AT/cm², about 50 pmol AT/cm², about 55 pmol AT/cm², about 60 pmol AT/cm², about 65 pmol AT/cm², or about 70 pmol AT/cm² as measured by an antithrombin (AT) binding assay.

The implant 100, when having a heparin coating as described herein, can have a ratio of the mass of the heparin coating to the total surface area of the implant 100 of between about 0.005 μg/mm² to about 0.011 μg/mm², about 0.007 μg/mm² to about 0.009 μg/mm², greater than about 0.005 μg/mm², greater than about 0.007 μg/mm², greater than about 0.008 μg/mm², less than about 0.015 μg/mm², less than about 0.009 μg/mm², about 0.008 μg/mm², or about 0.009 μg/mm².

The implant 100, when having a heparin coating as described herein, can have a ratio of the mass of the heparin coating to the abluminal surface area of the implant 100 of between about 0.01 μg/mm² to about 0.06 μg/mm², about 0.02 μg/mm² to about 0.05 μg/mm², about 0.03 μg/mm² to about 0.04 μg/mm², greater than about 0.01 μg/mm², greater than about 0.02 μg/mm², greater than about 0.03 μg/mm², less than about 0.06 μg/mm², less than about 0.05 μg/mm², about 0.03 μg/mm², about 0.035 μg/mm², or about 0.04 μg/mm².

The implant 100, when having a heparin coating as described herein, can have a ratio of the mass of the heparin coating to the wall thickness of the implant 100 of between about 0.005 μg/mm to about 0.015 μg/mm, about 0.007 μg/mm to about 0.014 μg/mm, about 0.008 μg/mm to about 0.013 μg/mm, greater than about 0.005 μg/mm, greater than about 0.007 μg/mm, greater than about 0.008 μg/mm, less than about 0.015 μg/mm, less than about 0.013 μg/mm, about 0.008 μg/mm, about 0.009 μg/mm, about 0.010 μg/mm, about 0.011 μg/mm, about 0.012 μg/mm, or about 0.013 μg/mm.

The implant 100, when having a heparin coating as described herein, can have a ratio of the thickness of the heparin coating to the wall thickness of the implant 100 (e.g., the tubular frame 120) of about 0.00005 or greater, such as about 0.00016 or greater.

The implant 100, when having a heparin coating as described herein, can have a ratio of the activity of the heparin coating to the wall thickness of the implant 100 of greater than about 0.30 pmol AT/cm²/μm, greater than about 0.35 pmol AT/cm²/μm, greater than about 0.40 pmol AT/cm²/μm, greater than about 0.45 pmol AT/cm²/μm, greater than about 0.50 pmol AT/cm²/μm, greater than about 0.55 pmol AT/cm²/μm, greater than about 0.60 pmol AT/cm²/μm, greater than about 0.65 pmol AT/cm²/μm, greater than about 0.70 pmol AT/cm²/μm, greater than about 0.75 pmol AT/cm²/μm, greater than about 0.80 pmol AT/cm²/μm, greater than about 0.85 pmol AT/cm²/μm, greater than about 0.90 pmol AT/cm²/μm, greater than about 0.95 pmol AT/cm²/μm, greater than about 1.00 pmol AT/cm²/μm, greater than about 1.10 pmol AT/cm²/μm, greater than about 1.15 pmol AT/cm²/μm, greater than about 1.20 pmol AT/cm²/μm, greater than about 1.25 pmol AT/cm²/μm, greater than about 1.30 pmol AT/cm²/μm, greater than about 1.35 pmol AT/cm²/μm, greater than about 1.40 pmol AT/cm²/μm, greater than about 1.45 pmol AT/cm²/μm, greater than about 1.50 pmol AT/cm²/μm, about 0.45 pmol AT/cm²/μm, about 0.50 pmol AT/cm²/μm, about 0.55 pmol AT/cm²/μm, about 0.60 pmol AT/cm²/μm, about 0.65 pmol AT/cm²/μm, about 0.70 pmol AT/cm²/μm, about 0.75 pmol AT/cm²/μm, about 0.80 pmol AT/cm²/μm, about 0.85 pmol AT/cm²/μm, about 0.90 pmol AT/cm²/μm, about 0.95 pmol AT/cm²/μm, about 1.00 pmol AT/cm²/μm, about 1.10 pmol AT/cm²/μm, about 1.15 pmol AT/cm²/μm, about 1.20 pmol AT/cm²/μm, about 1.25 pmol AT/cm²/μm, about 1.30 pmol AT/cm²/μm, or about 1.35 pmol AT/cm²/μm.

In some implementations, the implant 100 does not include a graft, a covering, or a liner. For example, in some implementations the implant 100 includes only a coating as described herein.

Table 2 below summarizes exemplary configurations and characteristics of implants 100 in accordance with some aspects of this disclosure.

TABLE 2
Exemplary Implant Configurations and Characteristics

| | | | | | Ratio of | Ratio of | Ratio of | |
|---|---|---|---|---|---|---|---|---|
| | | | | | Average | Mass of | Mass of | Ratio of |
| | | | | | Heparin | Heparin | Heparin | Mass of |
| | | | | | Coating | Coating | Coating | Heparin |
| | | | | | Activity to | to | to | Coating |
| | | Implant | Implant | | Implant | Implant | Implant | to Wall |
| Implant | Implant | Total | Abluminal | Implant | Wall | Total | Abluminal | thickness |
| Size | Wall | Surface | Surface | Abluminal | Thickness | Surface | Surface | of |
| (dia x | Thickness | Area | Area | Surface | (pmol | Area | Area | Implant |
| length, mm) | (μm) | (mm²) | (mm²) | Area (%) | AT/cm²/μm) | (μg/mm2) | (μg/mm2) | (μg/mm) |
| 3 × 15 | 43 | 39.9 | 9.54 | 8.08 | 0.8605 | 0.0087 | 0.0364 | 0.0081 |
| 3 × 20 | 43 | 50.64 | 12.64 | 8.11 | 0.8605 | 0.0087 | 0.0349 | 0.0103 |
| 3 × 23 | 43 | 58.01 | 14.2 | 8.12 | 0.8605 | 0.0087 | 0.0356 | 0.0117 |
| 4 × 20 | 43 | 52.17 | 12.51 | 5.79 | 0.8605 | 0.0087 | 0.0363 | 0.0106 |
| 4 × 23 | 43 | 62.83 | 14.56 | 5.81 | 0.8605 | 0.0087 | 0.0376 | 0.0127 |

### Apposition

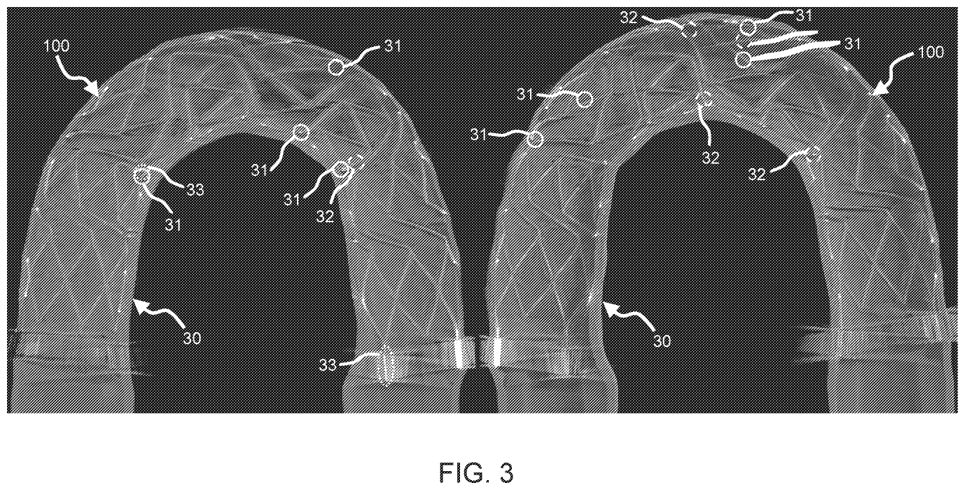

FIG. 3 shows an intraluminal implant 100 in accordance with FIGS. 2A-2E in an apposition bend test. The implant 100, which in this case has a diameter of 3 mm, is shown deployed centered inside a flexible silicone U-bent tube 30 having a bend radius of 4.9 mm and an inner diameter of 3 mm. The image at the left shows one half of the implant 100 within the U-bent tube and the image at the right shows the other half of the implant 100 within the U-bent tube. Encircled are locations of malapposition between the implant 100 and the inner wall of the U-bent silicone tube 30 in this test, with encircled locations 31 having a malapposition of less than 0.10 mm, encircled locations 32 having a malapposition of greater than or equal to 0.10 mm and less than 0.20 mm, and encircled locations 33 having a malapposition of greater than or equal to 0.20 mm. In this example, the implant 100 had 15 locations that were measured to have at least some malapposition between the implant 100 (e.g., a strut of the implant) and the inner wall of the U-bent silicone tube. The maximum measured malapposition between the implant 100 (e.g., a strut of the implant) and the inner wall of the U-bent silicone tube was 0.375 mm. Furthermore, the average measured malapposition between the implant 100 (e.g., a strut of the implant) and the inner wall of the U-bent silicone tube was 0.116 mm.

Implants as described herein (e.g., implant 100) can be configured to have about 50 or less, about 30 or less, about 25 or less, about 20 or less, about 15 or less, about 10 or less, or about 5 or less locations of at least some malapposition between the implant and a flexible silicone U-bent tube 30 as described in the apposition bend test above. Implants as described herein (e.g., implant 100) can be configured to have a maximum malapposition between the implant and a flexible silicone U-bent tube 30 as described in the apposition bend test above of about 1.00 mm or less, about 0.75 mm or less, about 0.50 or less, about 0.40 mm or less, about 0.375 mm or less, about 0.35 or less, about 0.325 mm or less, about 0.30 mm or less, about 0.275 mm or less, about 0.25 mm or less, about 0.225 mm or less, about 0.20 mm or less, about 0.175 mm or less, about 0.15 mm or less, about 0.125 mm or less, about 0.10 mm or less, about 0.075 mm or less, or about 0.05 mm or less. Furthermore, implants as described herein (e.g., implant 100) can be configured to have an average malapposition between the implant and a flexible silicone U-bent tube 30 as described in the apposition bend test above of 0.35 or less, about 0.325 mm or less, about 0.30 mm or less, about 0.275 mm or less, about 0.25 mm or less, about 0.225 mm or less, about 0.20 mm or less, about 0.175 mm or less, about 0.15 mm or less, about 0.125 mm or less, about 0.120 mm or less, about 0.115 mm or less, about 0.10 mm or less, about 0.075 mm or less, about 0.05 mm or less, or about 0.025 mm or less.

### Implant Variants

FIG. 4 illustrates an implant 400 that is a variant of the implant 100 described with respect to FIGS. 2A-2E. The implant 400 can be similar to the implant 100 in some or many respects. For example, the implant 400 can have a generally tubular frame 410 with a proximal end 401, a distal end 402, and a plurality of longitudinally spaced apart rings 441 that extend along a circumference of the tubular frame 410 the same or similar to the tubular frame 110 and the plurality of rings 141 of the implant 100. Each ring of the plurality of rings 441 of the implant 400 can include a plurality of ring struts 442, with adjacent pairs of ring struts joining at a plurality of proximal apexes 443 and a plurality of distal apexes 444 to form a chevron pattern as shown the same or similar to the plurality of rings 141, the plurality of ring struts 142, the plurality of proximal apexes 143, and the plurality of distal apexes 144 of the implant 100. Furthermore, the implant 400 can include a plurality of linking struts 445 that extend at least partially along the circumference of the tubular frame 410, with each linking strut of the plurality of linking struts 445 connecting a distal apex of one ring of the plurality of rings 441 to a proximal apex of an adjacent ring of the plurality of rings 441 as shown the same or similar to the linking struts 145 of the implant 100. The implant 400 can include a thromboresistant coating, such as a heparin coating, the same or similar to the coating that can be included on implant 100.

The implant 400 can differ from the implant 100 in that it can exclude a proximal portion having a ring and/or a distal portion having a ring as can be including in the implant 100 (e.g., proximal portion 120 with ring 121 and/or distal portion 160 with ring 161), although in some implementations the implant 400 can include such proximal and/or distal portions. The implant 400 can also differ from the implant 100 in that it can exclude flared ends/portions as can be included in the implant 100, although in some implementations the implant 400 can include such flared ends/portions. The implant 400 can differ from the implant 100 in that it can exclude one or more proximally extending struts and/or one or more distally extending struts along with radiopaque markers as can be included in the implant 100 (e.g., the one or more proximally extending struts 125, the one or more distally extending struts 165, and the radiopaque markers 181, 182), although in some implementations the implant 400 can include such one or more proximally extending struts, such one or more distally extending struts, and/or such radiopaque markers.

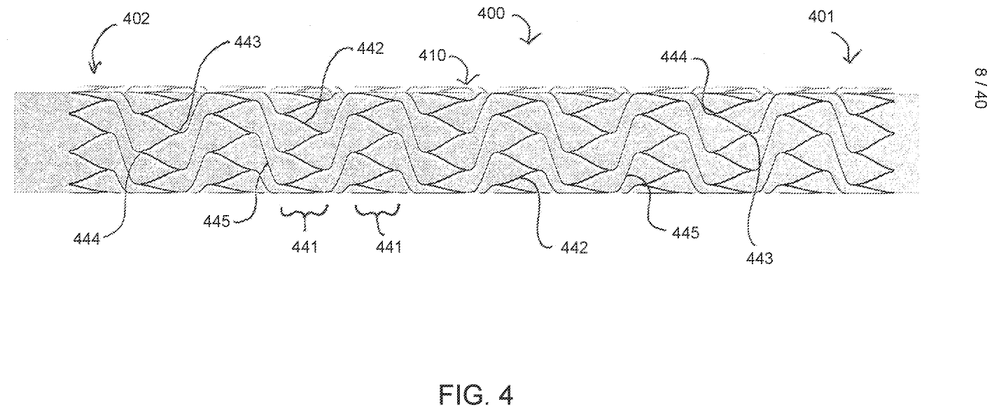

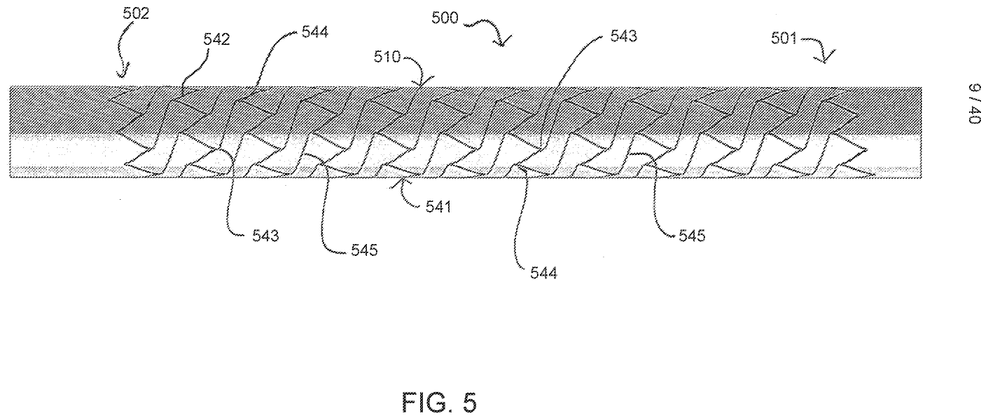

FIG. 5 illustrates an implant 500 that is a variant of the implant 100 described with respect to FIGS. 2A-2E. The implant 500 can be similar to the implant 100 in some or many respects. For example, the implant 500 can have a generally tubular frame 510 with a proximal end 501 and a distal end 502 the same or similar to the generally tubular frame 110 of implant 100. The implant 500 can differ from the implant 100 in that instead of having longitudinally spaced apart rings 141 (e.g., discrete rings spaced longitudinally along the length of the implant), the implant 500 can include a continuous ring 541 that revolves helically around the circumference of the tubular frame 510. The ring 541 can include a plurality of ring struts 542, with adjacent pairs of ring struts joining at a plurality of proximal apexes 543 and a plurality of distal apexes 544 to form a chevron pattern as shown. Furthermore, the implant 500 can include a plurality of linking struts 545 that extend at least partially along the circumference of the tubular frame 510, with each linking strut of the plurality of linking struts 545 connecting a distal apex of the plurality of distal apexes 544 to a proximal apex of the plurality of proximal apexes 543 similar to the linking struts 145 of the implant 100. The implant 500 can include a thromboresistant coating, such as a heparin coating, the same or similar to the coating that can be included on implant 100.

The implant 500 can further differ from the implant 100 in that it can exclude a proximal portion having a ring and/or a distal portion having a ring as can be including in the implant 100 (e.g., proximal portion 120 with ring 121 and/or distal portion 160 with ring 161), although in some implementations the implant 500 can include such proximal and/or distal portions. The implant 500 can also differ from the implant 100 in that it can exclude flared ends/portions as can be included in the implant 100, although in some implementations the implant 500 can include such flared ends/portions. The implant 500 can differ from the implant 100 in that it can exclude one or more proximally extending struts and/or one or more distally extending struts along with radiopaque markers as can be included in the implant 100 (e.g., the one or more proximally extending struts 125, the one or more distally extending struts 165, and the radiopaque markers 181, 182), although in some implementations the implant 500 can include such one or more proximally extending struts, such one or more distally extending struts, and/or such radiopaque markers.

### Delivery System

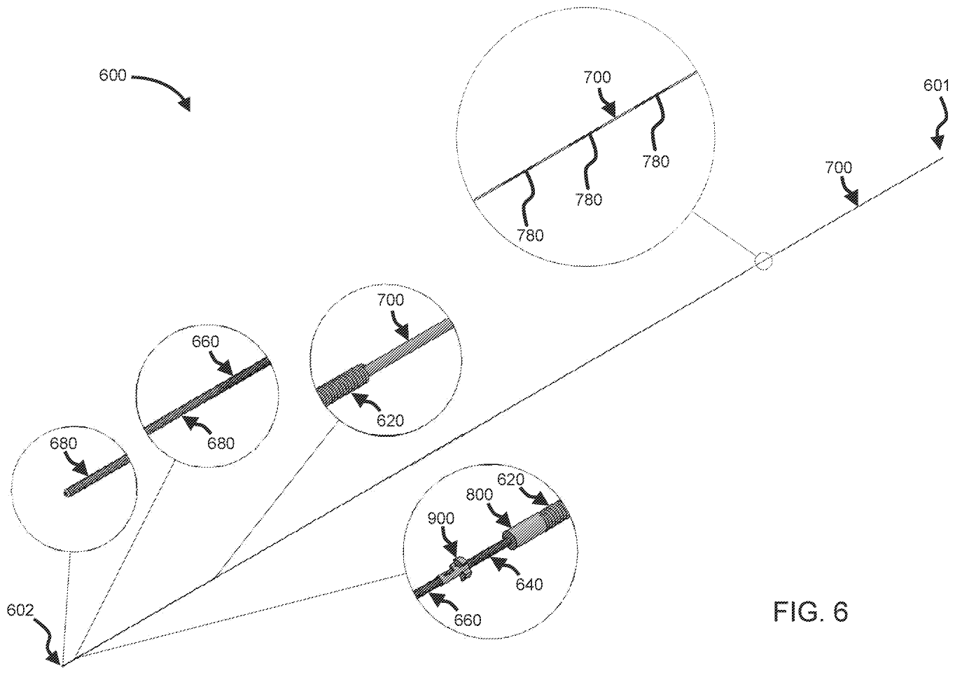

FIG. 6 illustrates a delivery wire 600 in accordance with some aspects of this disclosure. The delivery wire 600 can extend generally longitudinally between its proximal end 601 and its distal end 602. The deliver wire 600 can include a core wire 700, a proximal coil 620, a bumper 800, a distal coil 640, a coupler 900, a spacer coil 660, and a radiopaque coil 680 as shown. As will be described herein, the delivery wire 600 can be configured to travel through an implant delivery catheter and to deliver an implant as described herein, such as the implant 100.

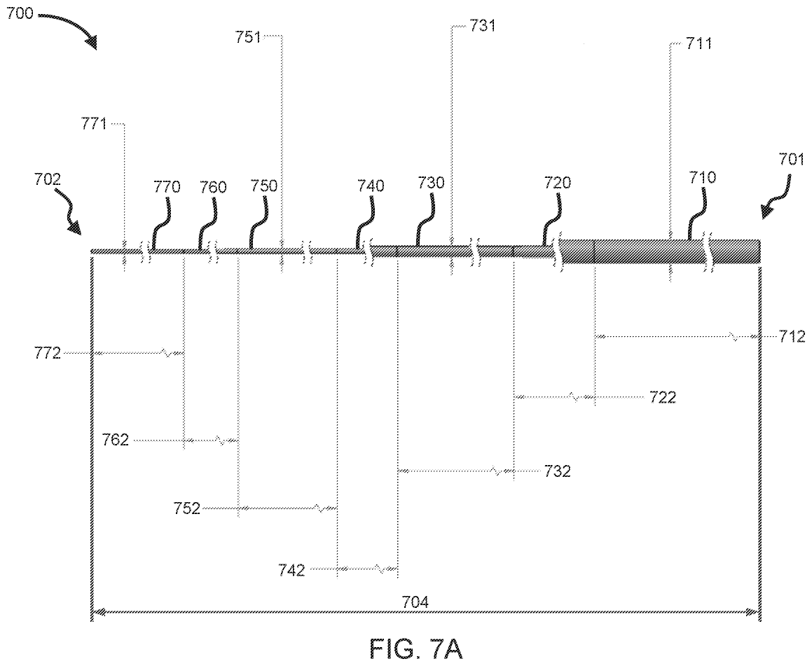

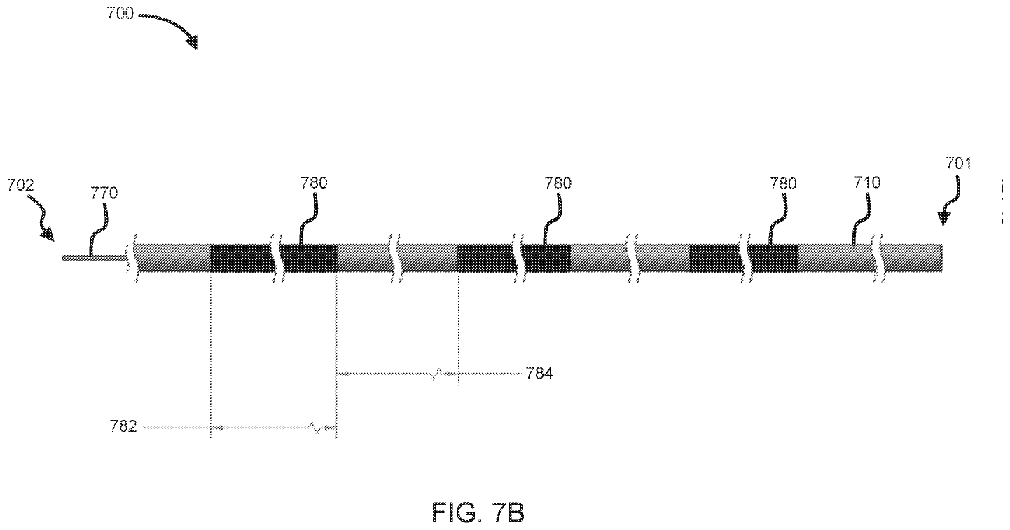

FIGS. 7A-7B illustrate side views of the core wire 700 of the delivery wire 600 of FIG. 6 in accordance with some aspects of this disclosure. The core wire 700 can extend generally longitudinally over its length 704 between its proximal end 701 and its distal end 702. The core wire 700 can include one or more substantially constant diameter sections and one or more tapered sections to produce a core wire 700 having a greater diameter at its proximal end 701 than at its distal end 702. For example and as shown in FIGS. 7A-7B, the core wire 700 can include a first constant diameter section 710 having a diameter 711 and a length 712, a first tapered section 720 having a length 722, a second constant diameter section 730 having a diameter 731 and a length 732, a second tapered section 740 having a length 742, a third constant diameter section 750 having a diameter 751 and a length 752, a third tapered section 760 having a length 762, and a fourth constant diameter section 770 having a diameter 771 and a length 772. As shown, the first constant diameter section 710 can extend distally from the proximal end 701 of the core wire 700, the first tapered section 720 can extend distally from the first constant diameter section 710, the second constant diameter section 730 can extend distally from the first tapered section 720, the second tapered section 740 can extend distally from the second constant diameter section 730, the third constant diameter section 750 can extend distally from the second tapered section 740, the third tapered section 760 can extend distally from the third constant diameter section 750, and the fourth constant diameter section 770 can extend distally from the third tapered section 760 and terminate at the core wire's distal end 702. In some implementations, the core wire 700 is a stainless steel spring wire (e.g., type 304 stainless steel) that is ground to create the one or more constant diameter sections and the one or more tapered sections.

The core wire 700 can have a length 704 of at least about 1000 mm. In some implementations, the core wire 700 has a length 704 of about 1900 mm. In such implementations, the length 712 of the first constant diameter section 710 can be about 1500 mm, the length 722 of the first tapered section can be about 60 mm, the length 732 of the second constant diameter section 730 can be about 200 mm, the length 742 of the second tapered section 740 can be about 40 mm, the length 752 of the third constant diameter section 750 can be about 88 mm, the length 762 of the third tapered section 760 can be about 4 mm, and the length 772 of the fourth constant diameter section 770 can be about 8 mm. The core wire 700 can have a maximum diameter of about 0.75 mm or less. In some implementations, the core wire 700 has a maximum diameter of about 0.3810 mm. In such implementations, the diameter 711 of the first constant diameter section 710 can be about 0.3810 mm, the diameter 731 of the second constant diameter section 730 can be about 0.1778 mm, the diameter 751 of the third constant diameter section 750 can be about 0.0762 mm, and the diameter 771 of the fourth constant diameter section 770 can be about 0.0559 mm. Furthermore, in such implementations the first tapered section 720 can taper over its length 722 from the diameter 711 of the first constant diameter section to the diameter 731 of the second constant diameter section, the second tapered section 740 can taper over its length 742 from the diameter 731 of the second constant diameter section to the diameter 751 of the third constant diameter section, and the third tapered section 760 can taper over its length 762 from the diameter 751 of the third constant diameter section to the diameter 771 of the fourth constant diameter section. Although exemplary lengths and diameters for the core wire 700 and its sections 710, 720, 730, 740, 750, 760, and 770 have been provided, any of such lengths and/or diameters can be less than or greater than those given, and/or such lengths and/or diameters can scale as the core wire is reduced or increased in length and/or diameter. While not shown, in some implementations the core wire 700 can include a lumen configured to receive a guidewire therethrough (and thus the delivery wire 600 can be configured to have a guidewire extend therethrough).

With reference to FIG. 7B, the core wire 700 can include one or more markers 780. The one or more markers 780 can be configured as one or more visual indicators useful for the delivery of an implant as described herein. For example, the core wire 700 can be laser marked to create the one or more markers 780. As shown in FIG. 7B, the core wire 700 can include three markers 780, although the core wire can include one, two, three, four, five, or more markers 780. Also shown in FIG. 7B, the markers 780 can each have a length 782 and can be spaced apart from one another by a length 784. In some implementations, the length 782 of the markers 780 can be between about 6 mm and about 14 mm (e.g., about 10 mm) with a spacing length 784 of between about 6 mm and about 14 mm (e.g., about 10 mm). The length 782 of the markers 780 can be the same or different, and the spacing length 784 therebetween can be the same or different. The markers 780 can be included on the first constant diameter section 710. For example, a distal-most end of a distal-most marker 780 can be positioned about 1350 mm distal of the proximal end 701 of the core wire 700.

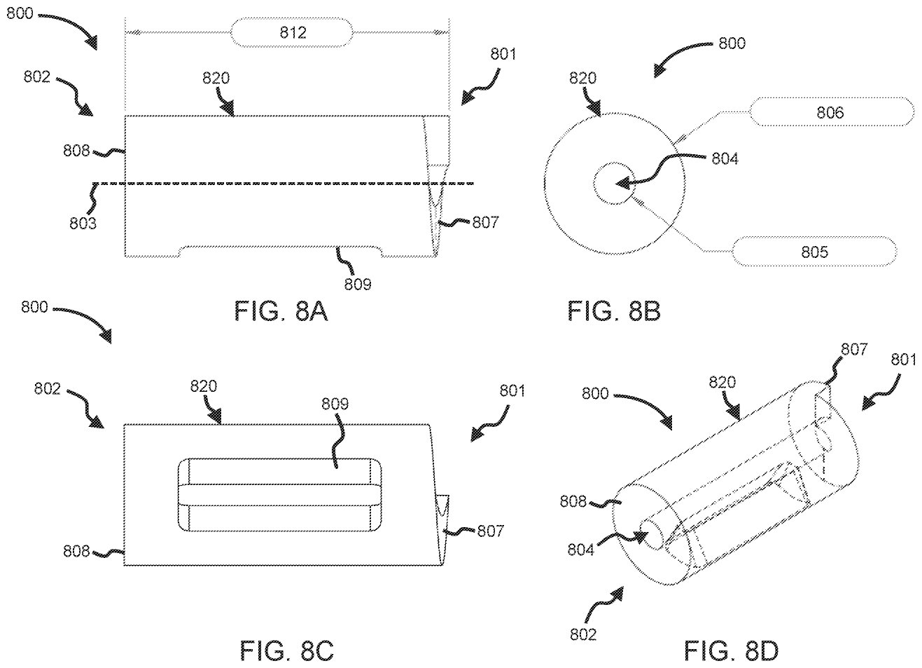

FIGS. 8A-8D illustrate a bumper 800 of the delivery wire 600 of FIG. 6 in accordance with some aspects of this disclosure. FIG. 8A shows a side view, FIG. 8B shows an end view, FIG. 8C shows another side view, and FIG. 8D shows a perspective view of the bumper 800. The bumper 800 can have a generally tubular body 820 having a proximal end 801, a distal end 802, a length 812 and a longitudinal axis 803 extending between the proximal end 801 and the distal end 802, an outer diameter 806, and an inner diameter 805 defining a lumen 804. As shown, the bumper 800 can have a helical cut face 807 at its proximal end 801. Such helical cut face 807 can be configured to mate with an end of a coil, such as a distal end of the proximal coil 620. Further as shown, the bumper 800 can have a substantially flat face 808 at its distal end 802. The bumper 800 can be configured to attach to the core wire 700, for example, over the core wire 700. For such attachment, the bumper 800 can have a through hole 809 that extends from the inner diameter 805 and through the outer diameter 806 (e.g., through a thickness of the tubular body 820 of the bumper 800) along a side of the bumper 800. The through hole 809 can be configured for a weld to attach the bumper 800 to the core wire 700.

The length 812 of the bumper 800 can be between about 0.500 mm and about 0.9 mm, for example about 0.762 mm. The outer diameter 806 of the bumper 800 can be between about 0.200 mm and about 0.400 mm, for example about 0.330 mm. The inner diameter 805 of the bumper 800 can be between about 0.070 mm and about 0.130 mm, for example about 0.0965 mm. The bumper 800 can be made of stainless steel (e.g., 304 stainless steel).

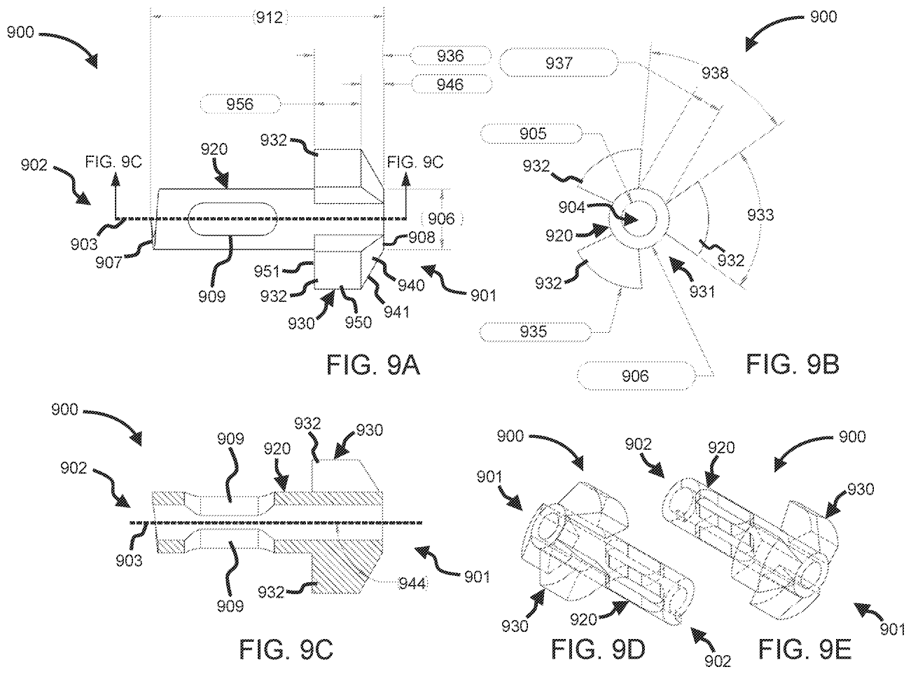

FIGS. 9A-9E illustrate a coupler 900 of the delivery wire 600 of FIG. 6 in accordance with some aspects of this disclosure. FIG. 9A shows a side view, FIG. 9B shows an end view, FIG. 9C shows a cross-sectional side view as indicated in FIG. 9A, and FIGS. 9D-9E show perspective views of the coupler 900. The coupler 900 can have a generally tubular body 920 having a proximal end 901, a distal end 902, a length 912 and a longitudinal axis 903 extending between the proximal end 901 and the distal end 902, an outer diameter 906, an inner diameter 905 defining a lumen 904, and a hub 930 adjacent the proximal end 901. The tubular body 920 of the coupler 900 can have a helical cut face 907 at its distal end 902. Such helical cut face 907 can be configured to mate with an end of a coil, such as a proximal end of the spacer coil 660. Further as shown, the tubular body 920 of the coupler 900 can have a substantially flat face 908 at its proximal end 901. The coupler 900 can be configured to attach to the core wire 700, for example, over the core wire 700. For such attachment, the coupler 900 can have one or more through holes 909 that extend from the inner diameter 905 and through the outer diameter 906 (e.g., through a thickness of the tubular body 920 of the coupler 900) along a side of the coupler 900. The one or more through holes 909 can be configured for a weld to attach the coupler 900 to the core wire 700.

The coupler 900 can be configured for releasable engagement with an implant as described herein, such as the implant 100, for delivery of the implant. For this, the hub 930 can extend radially outward of the outer diameter 906 of the tubular body 920 and have one or more slots 931 configured to releasably receive therein at least a portion of an implant as described herein. For example, the one or more slots 931 can be configured to releasably receive therein the neck portions 126 of the one or more proximally extending struts 125 of the implant 100 (e.g., each slot can receive a neck portion of a proximally extending strut).

With continued reference to FIGS. 9A-9E, the one or more slots 931 of the hub 930 can have a width 937 at the outer diameter 906 of the tubular body 920 and have a slot angle 938. The one or more slots 931 in the hub 930 can define hub portions 932 having a hub portion angle 933. The hub 930 can have a hub diameter 935 and a hub length 936. Furthermore, the hub 930 can have a proximal portion 940 having a proximal portion length 946 and a proximal face 941 that can be at an angle 944 relative to the longitudinal axis 903, and a distal portion 950 having a distal portion length 956 and a distal face 951. The angle 944 of the proximal face 941 can advantageously aid in retraction of the delivery wire 600 within a catheter after it has been extended distally therefrom (e.g., the angle can help prevent catching of the delivery wire 600 on a catheter tip), such as will be described with respect to delivery of an implant. The hub 930 can have three hub portions 932 and three slots 931 therebetween as shown, however the hub 930 can be configured to have less than three of each or more than three of each.

The length 912 of the coupler 900 can be between about 0.300 mm and about 1.000 mm, for example about 0.635 mm. The outer diameter 906 of the tubular body 920 of the coupler 900 can be between about 0.065 mm and about 0.265 mm, for example about 0.165 mm. The inner diameter 905 of the tubular body 920 of the coupler 900 can be between about 0.05 mm and about 0.1965 mm, for example about 0.0965 mm. The diameter 935 of the hub 930 can be between about 0.200 mm and about 0.500 mm, for example about 0.381 mm. The length 936 of the hub 930 can be between about 0.050 mm and about 0.400 mm, for example about 0.178 mm. The width 937 of the one or more slots 931 at the outer diameter 906 of the tubular body 920 can be between about 0.030 mm and about 0.130 mm, for example about 0.086 mm. The slot angle 938 of the one or more slots 931 can be between about 20 degrees and about 80 degrees, for example about 50 degrees. The hub portion angle 933 of the hub portions 932 can be between about 40 degrees and about 110 degrees, for example about 70 degrees. The proximal portion length 946 of the proximal portion 940 can be between about 0.020 mm and about 0.080 mm, for example about 0.051 mm. The angle 944 of the proximal face 941 can be between about 15 degrees and about 115 degrees, for example about 60 degrees, relative to the longitudinal axis 903. The distal portion length 956 of the distal portion 950 can be between about 0.020 mm and about 0.230 mm, for example about 0.127 mm. The coupler 900 can be made of stainless steel (e.g., 304 stainless steel).

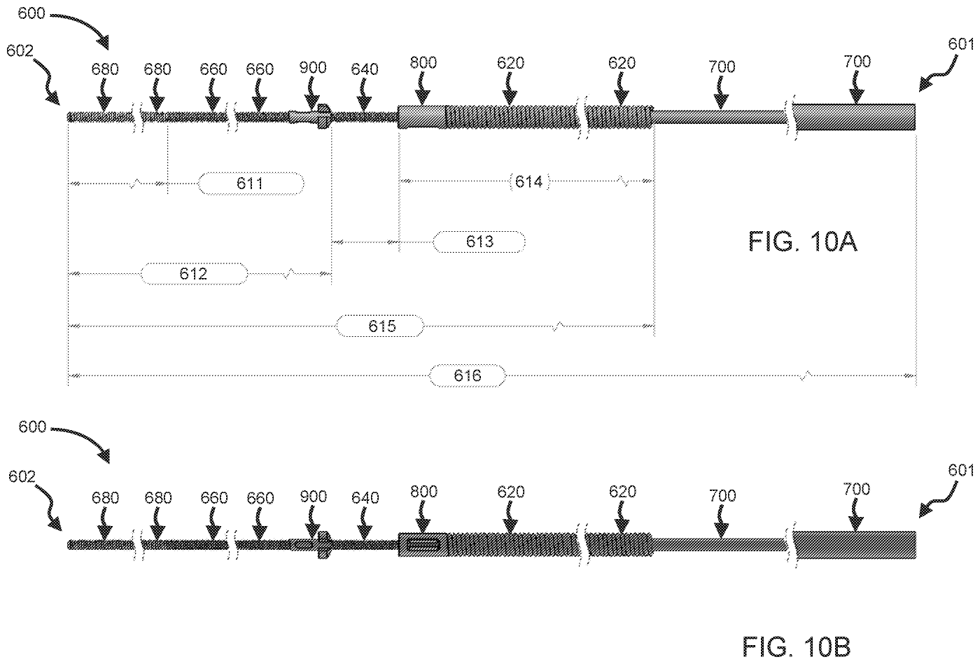

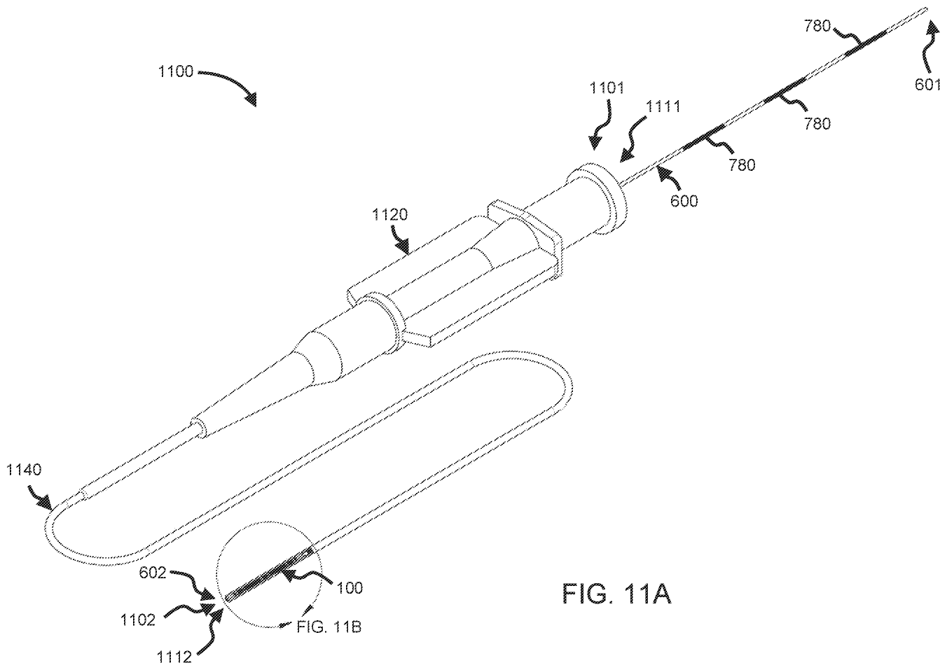

FIGS. 10A-10B illustrate side views of the delivery wire 600 of FIG. 6 in accordance with some aspects of this disclosure. As previously mentioned, the delivery wire 600 can include the core wire 700 described with respect to FIGS. 7A-7B, the proximal coil 620, the bumper 800 described with respect to FIGS. 8A-8D, the distal coil 640, the coupler 900 described with respect to FIGS. 9A-9E, the spacer coil 660, and the radiopaque coil 680 as shown. The core wire 700 can extend through each of the proximal coil 620, the bumper 800, the distal coil 640, the coupler 900, the spacer coil 660, and the radiopaque coil 680.

The proximal coil 620 can be made of stainless steel (e.g., type 304 stainless steel) spring wire having a wire diameter of about 0.0635 mm and wound into a coil having an inner diameter of about 0.203 mm and an outer diameter of about 0.381 mm. The length of the proximal coil 620 can between about 200 mm and about 400 mm, for example about 292 mm.

The distal coil 640 can be made of stainless steel (e.g., type 304 stainless steel) spring wire having a wire diameter of about 0.025 mm and wound into a coil having an inner diameter of about 0.076 mm and an outer diameter of about 0.152 mm. The length of the distal coil 640 can be between about 0.500 mm and about 1.500 mm, for example about 1.02 mm. In some embodiments, the length of the distal coil 640 can be about or greater than about a length of the proximal radiopaque markers 181 and/or a length of the connection portion 127 of the one or more proximally extending struts 125 of the implant 100.

The spacer coil 660 can be made of stainless steel (e.g., type 304 stainless steel) spring wire having a wire diameter of about 0.025 mm and wound into a coil having an inner diameter of about 0.076 mm and an outer diameter of about 0.152 mm. The length of the spacer coil 660 can be between about 2.000 mm and about 10.000 mm, for example about 3.277 mm, about 5.461 mm, or about 6.807 mm. In some embodiments, the length of the spacer coil 660 can be adjusted based on the length of the implant, such as the implant 100.

The radiopaque coil 680 can be made of a radiopaque material (e.g., 92/8 platinum-tungsten) wire having a wire diameter of about 0.030 mm and wound into a coil having an inner diameter of about 0.076 mm and an outer diameter of about 0.152 mm. The length of the radiopaque coil 680 can be between about 10.000 mm and about 35.000 mm, for example about 17.221 mm, about 21.387 mm, about 22.631 mm, or about 25.121 mm. In some embodiments, the length of the radiopaque coil 680 can be adjusted based on the length of the implant, such as the implant 100. In some embodiments, the length of the radiopaque coil 680 can be configured to be about the same length of the implant 100 after it is deployed inside the vessel 5, which can include a foreshortened length of the implant 100. In such embodiments, the length of the spacer coil 660 can correspondingly be adjusted based on the length of the radiopaque coil 680 and the configuration of the implant 100 (e.g., the length and diameter of implant 100).

With continued reference to FIGS. 10A-10B, a proximal end of the proximal coil 620 can attach to (e.g., be welded to) the core wire 700, with the core wire 700 extending through the proximal coil 620. In some embodiments, the proximal end of the proximal coil 620 attaches to the core wire 700 along the second constant diameter section 730 of the core wire 700. A distal end of the proximal coil 620 can attach to the proximal end 801 of the bumper 800, with the core wire 700 extending through the lumen 804 of the bumper 800. For example, the distal end of the proximal coil 620 can mate with the helical cut face 807 at the proximal end 801 of the bumper 800 and attach thereto (e.g., be welded thereto). The bumper 800 can attach to (e.g., be welded to) the core wire 700 via the through hole 809. In some embodiments, the bumper 800 attaches to the core wire 700 along the third constant diameter section 750 of the core wire 700. The distal end 802 of the bumper 800 can be positioned adjacent a proximal end of the distal coil 640, with the core wire 700 extending through the distal coil 640. For example, the proximal end of the distal coil 640 can rest against the flat face 808 at the distal end 802 of the bumper 800. The proximal end 901 of the coupler 900 can be positioned adjacent a distal end of the distal coil 640, with the core wire 700 extending through the lumen 904 of the coupler 900. For example, the flat face 908 at the proximal end 901 of the coupler 900 can rest against the distal end of the distal coil 640. The coupler 900 can attach to (e.g., be welded to) the core wire 700 via the one or more through holes 909. In some embodiments, the coupler 900 attaches to the core wire 700 along the third constant diameter section 750 of the core wire 700. A proximal end of the spacer coil 660 can attach to (e.g., be welded to) the distal end 902 of the coupler 900, with the core wire 700 extending through the spacer coil 660. For example, the proximal end of the spacer coil 660 can mate with the helical cut face 907 at the distal end 902 of the coupler 900 and attach thereto (e.g., be welded thereto). A proximal end of the radiopaque coil 680 can attach to (e.g., be welded to) the distal end of the spacer coil 660, with the core wire 700 extending through the radiopaque coil 680. The distal end of the radiopaque coil 680 can be attached to (e.g., be welded to) the distal end 702 of the core wire 700. For example, the distal end of the radiopaque coil 680 can form a bullet nosing end with the distal end 702 of the core wire 700 to create a rounded, tapered distal end 602 of the delivery wire 600. In some embodiments, the radiopaque coil 680 attaches to the core wire 700 at the distal end of the fourth constant diameter section 770 of the core wire 700.

As shown in FIGS. 10A-10B, the proximal coil 620 can attach to the core wire 700 at a length 615 from the distal end 602 of the delivery wire 600. The length 615 can be at least about 200 mm or at least about 300 mm, for example about 312 mm. The proximal coil 620 and the bumper 800, when mated as shown, can have a combined length 614 of between about 150 mm and about 315 mm, for example about 292 mm. The distal end 802 of the bumper 800 and the proximal end 901 of the coupler 900 can be separated by a length 613, which can be the same as the length of the distal coil 640. The length 613 can be between about 0.500 mm and about 1.500 mm, for example about 1.02 mm. In some embodiments, the length 613 can be about or greater than about a length of the proximal radiopaque markers 181 and/or a length of the connection portion 127 of the one or more proximally extending struts 125 of the implant 100. The portion of the delivery wire 600 between the proximal end 901 of the coupler 900 and the distal end 602 of the delivery wire 600 can have a length 612. The length 612 can be between about 15.00 mm and about 30.00 mm, for example about 21.08 mm. The length 611 can be the same as the length of the radiopaque coil 680, which can be between about 10.000 mm and about 35.000 mm, for example about 17.221 mm, about 21.387 mm, about 22.631 mm, or about 25.121 mm. The lengths 612 and 611 (and thus the lengths of the coupler 900, the spacer coil 660 and/or the radiopaque coil 680) can be adjusted based on the length of an implant the delivery wire 600 is configured to deliver, for example the implant 100 described herein. The length 616 can be the same as the length of the core wire 700, which can be at least about 1000 mm, for example about 1900 mm. While examples of the configurations, connections, and relative positioning of the components of the delivery wire 600 have been described above, it is intended that modifications to such configurations, connections and relative positioning fall within the scope of this disclosure.

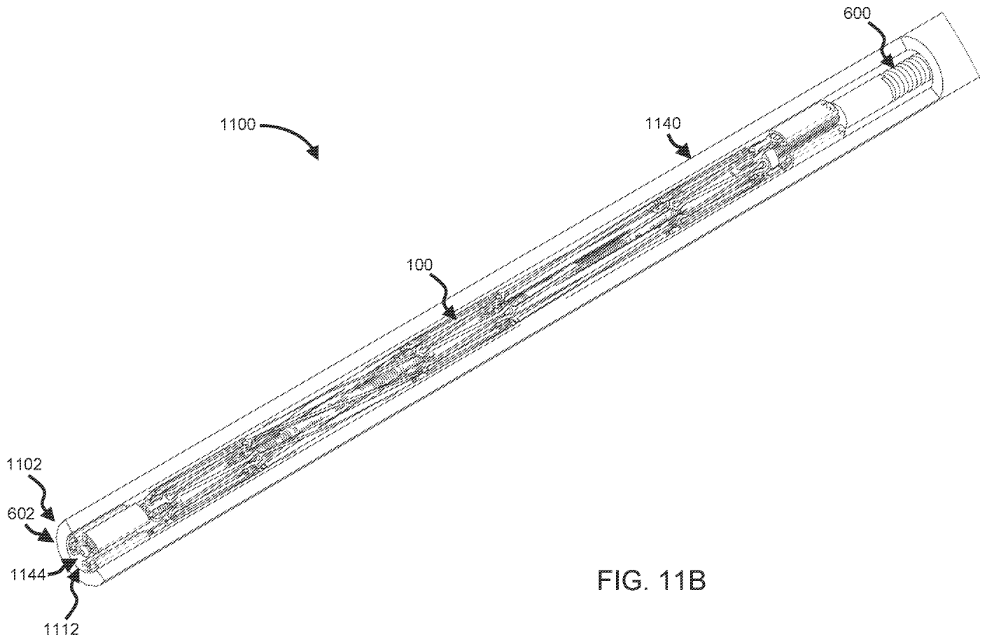

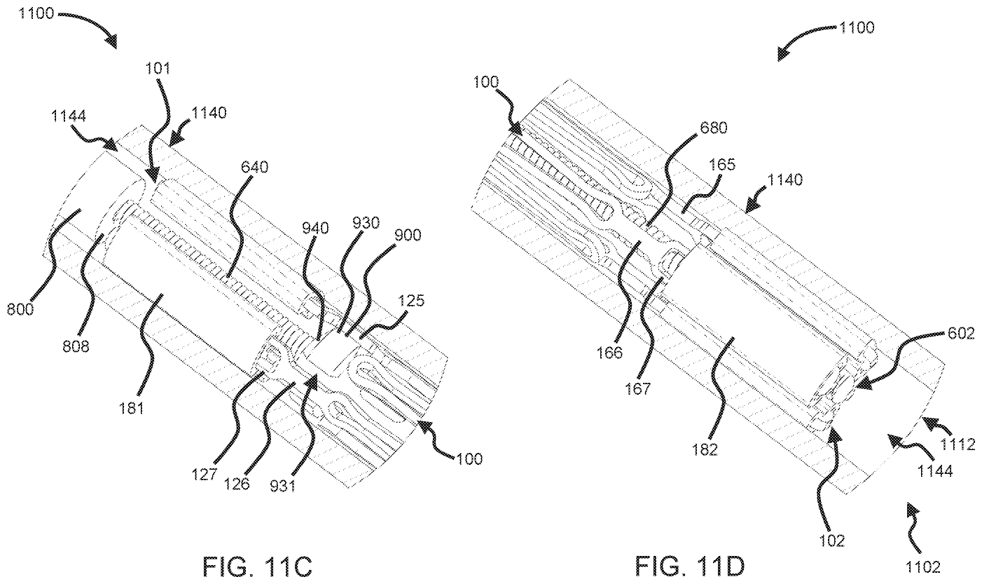

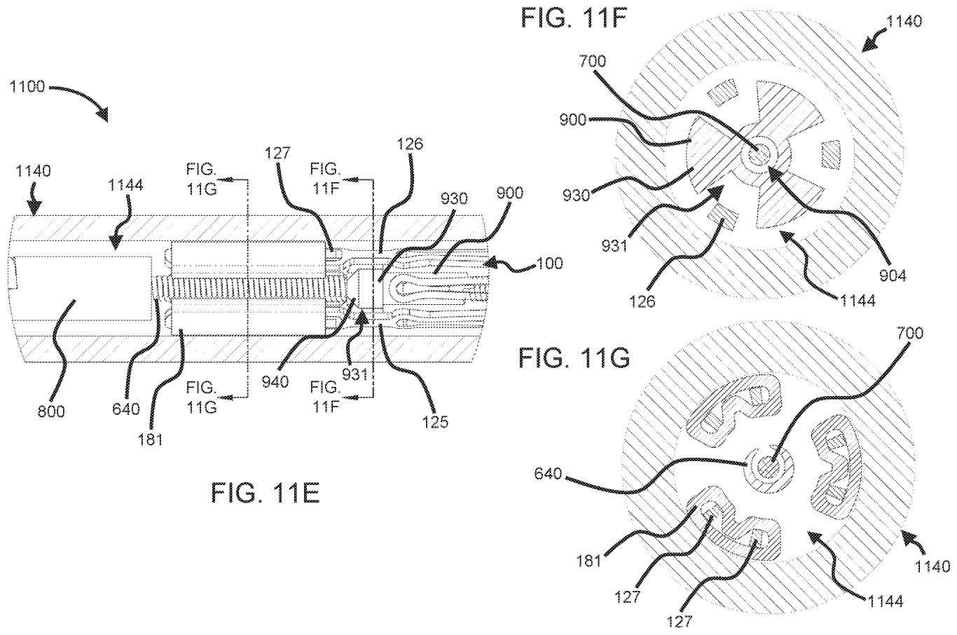

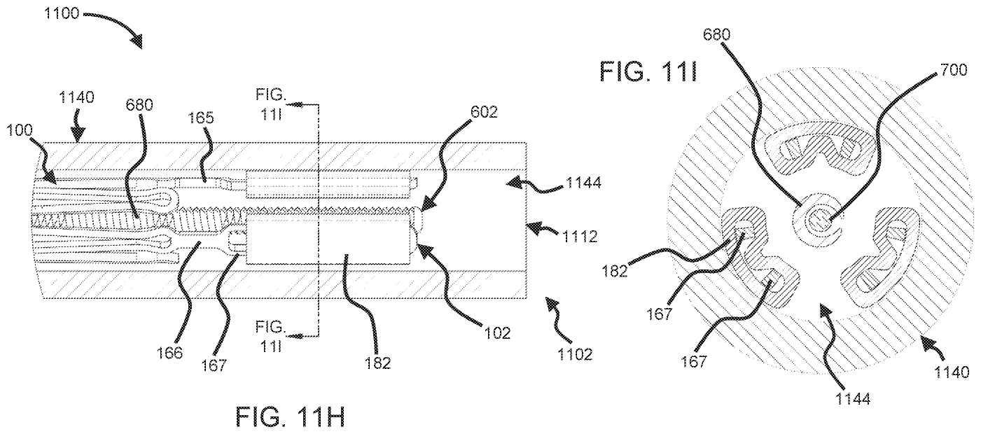

FIGS. 11A-11I illustrate an intraluminal implant delivery system 1100 in accordance with some aspects of this disclosure. The intraluminal implant delivery system 1100, which can also be referred to herein as an implant delivery system or implant deployment system, can include the delivery wire 600 described herein, an implant as described herein (e.g., implant 100), and a catheter 1140. FIG. 11A shows a perspective view of the implant delivery system 1100. FIG. 11B shows a close-up perspective view of the distal end of the implant delivery system 1100 with a portion of the wall of the catheter 1140 removed from view. FIGS. 11C-11D show close-up perspective views of the proximal end 101 and the distal end 102, respectively, of the implant 100 in a collapsed configuration within the implant delivery system 1100 with a portion of the wall of the catheter 1140 removed from view. FIGS. 11E-11G show side and related cross-sectional views of the proximal end 101 of the implant 100 in a collapsed configuration within the implant delivery system 1100. FIGS. 11H-11I show a side and related cross-sectional view of the distal end 102 of the implant 100 in a collapsed configuration within the implant delivery system 1100.

With reference to FIG. 11A, the catheter 1140 can have a generally tubular body with a lumen 1144 extending between an access port 1111 at its proximal end 1101 and an exit port 1112 at its distal end 1102. As shown, the catheter 1140 can include a hub 1120 adjacent the access port 1111 at its proximal end 1101. The access port 1111 can be configured to attach to a hemostatic valve, such as a rotating hemostatic valve (not shown). The lumen 1144 of the catheter 1140 can have a greater diameter at its proximal end, such as through at least a portion of the access port 1111 and/or through at least a portion of the hub 1120, and narrow to a smaller diameter within the access port 1111 or the hub 1120 or distal of the hub 1120. In some embodiments, the lumen 1144 can have substantially the same diameter along the entire length of the catheter 1140 including through the access port 1111 and the hub 1120. For example, the lumen 1144 of the catheter 1140 can have an internal diameter of between about 0.300 mm to about 0.600 mm, such as about 0.419 mm. An outer diameter of the catheter 1140 distal of the hub 1120 can vary along the length of the catheter or it can be substantially the same along its length. For example, the outer diameter of the catheter 1140 distal of the hub 1120 can be between about 0.400 mm to about 1.000 mm, such as about 0.787 mm near its proximal end distal of the hub 1120 and about 0.610 mm at its distal end 1102. The effective length of the catheter 1140 (e.g., the length of the catheter 1140 distal of the hub 1120) can be between about 100 cm and 200 cm, for example about 150 cm. Furthermore, the catheter 1140 can be a hybrid structure having one or more braided sections and/or one or more coiled sections making up the wall of the catheter 1140. While example configurations of the catheter 1140 have been described above, it is intended that modifications to such configurations fall within the scope of this disclosure. For example, the catheter 1140 can be configured to maintain an implant 100 in a collapsed configuration over the delivery wire 600 while the implant 100 and the delivery wire 600 are within the lumen 1144 of the catheter 1140. As another example, the catheter 1140 can be configured to access a neurovascular site of a subject 1 for delivery of an implant 100.

As mentioned above, FIG. 11B shows a close-up of the distal end 1102 of the implant delivery system 1100 with the implant 100 in its collapsed configuration over the delivery wire 600 and within the lumen 1144 of the catheter 1140 with a portion of the wall of the catheter 1140 removed for clarity. As shown, the lumen 1144 of the catheter 1140 can be configured to work with the delivery wire 600 to keep the implant 100 in its collapsed configuration.

FIGS. 11C-11I further show the interaction between the implant 100, the delivery wire 600, and the catheter 1140 of the implant delivery system 1100. As shown in FIG. 11C and FIGS. 11E-11F, the hub 930 of the coupler 900 can be configured to receive at least a portion of the implant 100. For example, the one or more slots 931 of the hub 930 can be configured to receive the neck portion 126 of the one or more proximally extending struts 125 of the implant 100. With such configuration, as the delivery wire 600 is moved (e.g., longitudinally) relative to the lumen 1144 of the catheter 1140, the implant 100 is made to move along with the delivery wire 600 by the interaction between the coupler 900 (e.g., the hub 930 of the coupler 900), the implant 100 (e.g., the neck portion 126 of the one or more proximally extending struts 125 of the implant 100), and the catheter 1140 (e.g., the lumen 1144 of the catheter 1140). To explain further, the hub diameter 935 of the hub 930 and the lumen 1144 can be configured to prevent the neck portion 126 of the one or more proximally extending struts 125 of the implant 100 from moving out of the one or more slots 931 of the hub 930 while the hub 930 is disposed within the lumen 1144. Furthermore, the one or more proximally extending struts 125 of the implant 100 can be configured to widen distal and proximal to the neck portion 126 such that the distal face 951 and proximal face 941 of the hub 930 can push against such widened portions upon distal or proximal movement of the delivery wire 600 relative to the lumen 1144. Thus, as the delivery wire 600 is moved distally and/or proximally relative to the catheter 1140 while the hub 930 is located within the lumen 1144, the implant 100 correspondingly moves distally and/or proximally.

In some implementations, the distal face 951 of the hub 930 can interact with other portions of the implant 100 to move the implant 100 distally when the delivery wire 600 is moved distally relative to the catheter 1140. Alternatively, or in combination, the bumper 800 (e.g., the flat face 808 of the bumper 800) can interact with a portion of the implant 100 (e.g., the connector portion 127 of the one or more proximally extending struts 125 and/or the proximal radiopaque markers 181) to move the implant 100 distally when the delivery wire 600 is moved distally relative to the catheter 1140. In some implementations, the proximal face 941 of the hub 930 can interact with other portions of the implant 100 to move the implant 100 proximally when the delivery wire 600 is moved proximally relative to the catheter 1140. For example, the proximal face 941 can interact with the connector portion 127 of the one or more proximally extending struts 125 and/or the proximal radiopaque markers 181 to move the implant 100 proximally when the delivery wire 600 is moved proximally relative to the catheter 1140.

With continued reference to FIGS. 11A-11H, portions of the delivery wire 600 can be configured to pass through the implant 100 (e.g., the lumen 104 of the implant 100) when the implant 100 is collapsed thereupon and the neck portion 126 of the one or more proximally extending struts 125 are disposed within the one or more slots 931 of the coupler 900. For example, FIGS. 11C, 11E, and 11G show the connector portion 127 of the one or more proximally extending struts 125 of the implant 100 and proximal radiopaque markers 181 connected thereto collapsed about the delivery wire (e.g., collapsed about the distal coil 640 and the core wire 700 extending therethrough) between the coupler 900 (e.g., the proximal face 941 of the hub 930 of the coupler 900) and the bumper 800 (e.g., the distal flat face 808 of the bumper 800). As another example, FIGS. 11D, 11H, and 11I show portions of the implant 100 (e.g., some or all portions of the implant 100 other than the one or more proximally extending struts 125) collapsed about the coupler 900, the spacer coil 660, and the radiopaque coil 680. As shown in these same figures, the distal end of the 102 of the implant 100 can be substantially aligned with the distal end 602 of the delivery wire 600 when the implant 100 is collapsed about the delivery wire 600 and within the lumen 1144 of the catheter 1140.

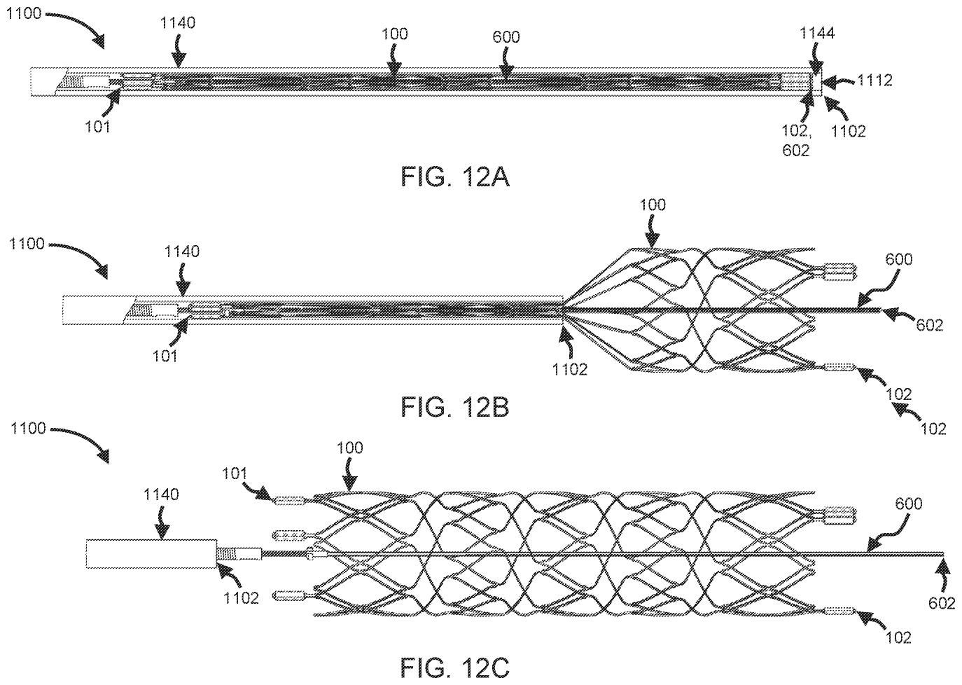

FIGS. 12A-12C illustrate delivery of the intraluminal implant 100 via the delivery wire 600 and the catheter 1140. FIG. 12A shows the implant 100 collapsed about the delivery wire 600 with distal ends 102 and 602 of the implant 100 and delivery wire 600, respectfully, substantially aligned with one another (e.g., in the longitudinal direction) and adjacent the exit port 1112 of the catheter 1140. This can be the relative positioning used during advancement of the implant delivery system 1100 within the subject 1 to the desired site of implantation. FIG. 12B shows the implant 100 partially expanded with a portion of the implant 100 (e.g., starting with its distal end 102) extending distally from the exit port 1112 at the distal end 1102 of the catheter 1140. Also shown is at least a portion of the delivery wire 600 (e.g., starting with its distal end 602) extending distally from the exit port 1102 of the catheter 1140. To attain such partial expansion of the implant 100, the delivery wire 600 can be moved distally relative to the catheter 1140 and/or the catheter 1140 can be moved proximally relative to the delivery wire 600. In some implementations, it is advantageous to simultaneously move the catheter 1140 proximally and the delivery wire 600 distally during implant 100 deployment. In the partially expanded state (which can also be referred to herein as a partially deployed state) shown in FIG. 12B, relative movement between the delivery wire 600 and the catheter 1140 can either withdraw the implant 100 back into the catheter 1140 or continue on and eventually release the implant 100 altogether from the exit port 1112 of the catheter 1140 and from the delivery wire 600 as shown in FIG. 12C. As will be discussed herein, the implant delivery system 1100 can be configured such that the implant 100 can be able to be withdrawn within the lumen 1144 of the catheter 1140 (e.g., for adjusting position or removing from the subject 1) while the hub 930 of the coupler 900 of the delivery wire 600 stays within the lumen 1144 of the catheter 1140 and/or at least a portion of the one or more proximally extending struts 125 stays within the lumen 1144 of the catheter 1140.

In some implementations, it is desirable to advance the implant delivery system 1100 within the subject 1 with the distal end 102 of the implant 100 and the distal end 602 of the delivery wire 600 spaced apart from the exit port 1112. For example, it can be desirable to advance the implant delivery system 1100 within the subject 1 with the distal end 102 of the implant 100 and the distal end 602 of the delivery wire 600 recessed within the lumen 1144 of the catheter 1140. Such relative positioning of the implant 100 and delivery wire 600 with the catheter 1140 can advantageously allow the distal tip of the catheter 1140 to at least partially deflect while traversing through the subject. In some implementations, it is desirable to advance the implant delivery system 1100 within the subject 1 with the distal end 102 of the implant 100 and the distal end 602 of the delivery wire 600 substantially aligned with the exit port 1112. In some embodiments, the implant delivery system 1100 can be advanced within the subject 1 while the implant 100 and delivery wire 600 are moved proximally and/or distally relative to the exit port 1112 of the catheter 1140 to tune a flexibility of the distal tip or distal portion of the catheter 1140 during advancement. Such tuning of the flexibility during advancement can improve the ability of the implant delivery system 1100 to navigate vessels of the subject 1.

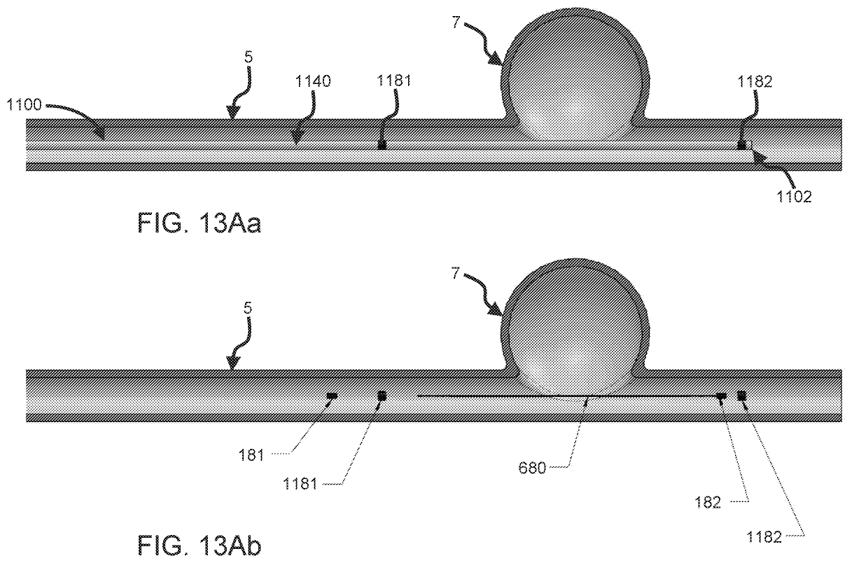

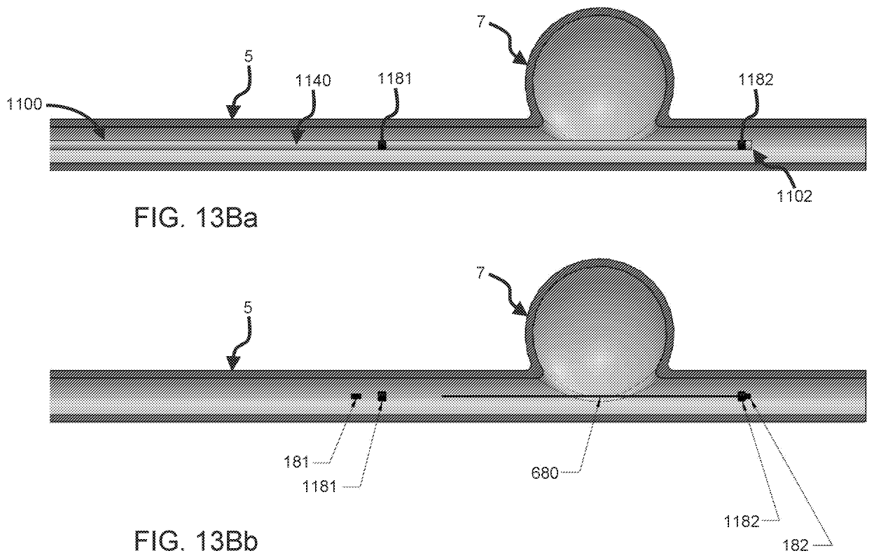

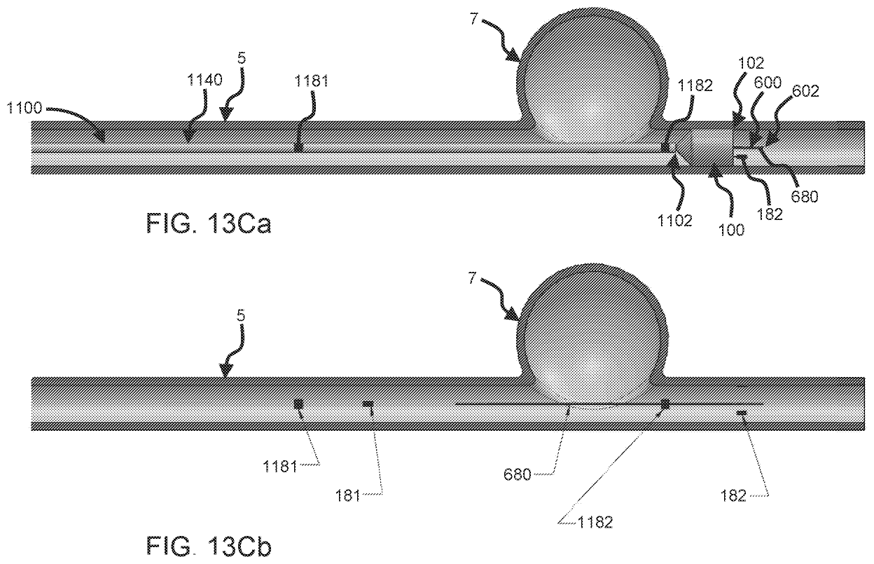

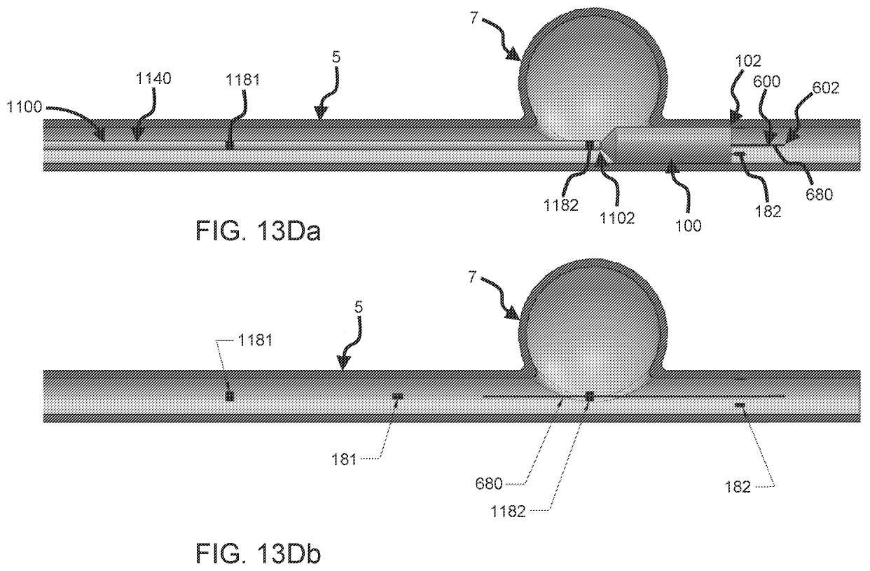

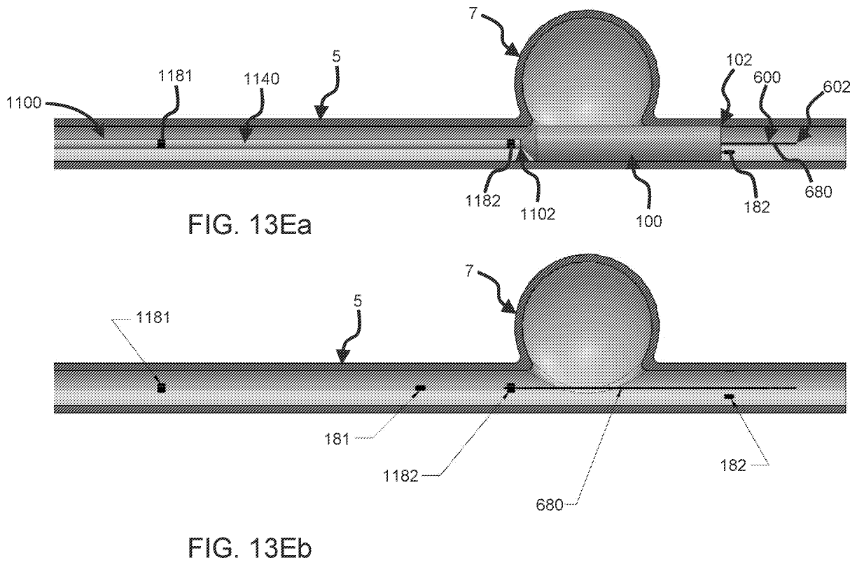

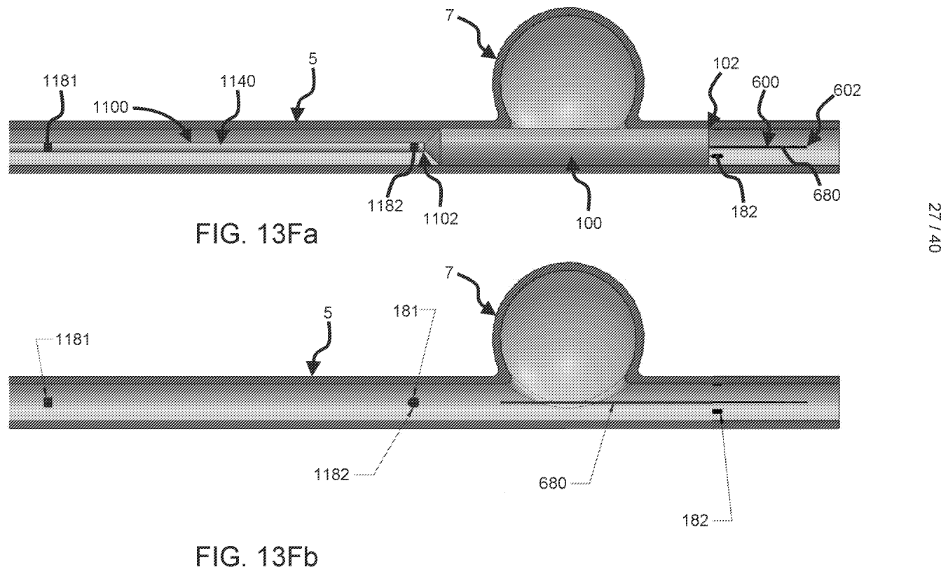

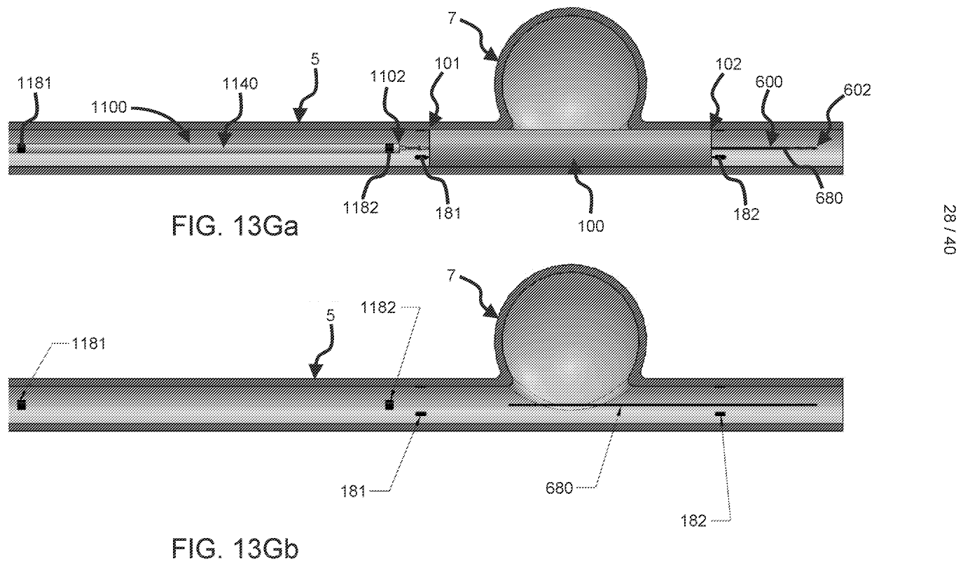

FIGS. 13Aa-13Gb illustrate delivery of an intraluminal implant 100 adjacent an aneurysm 7 in a vessel 5 in accordance with some aspects of this disclosure. Throughout FIGS. 13Aa-13Gb, the top panel (e.g., 13Aa, 13Ba, 13Ca, 13Da, 13Ea, 13Fa, 13Ga) shows an open cross-section of the vessel 5 in which the implant 100 is being deployed and a schematic representation of the implant delivery system 1100 (e.g., the catheter 1140, the delivery wire 600, and the implant 100), and the bottom panel (e.g., 13Ab, 13Bb, 13Cb, 13Db, 13Eb, 13Fb, 13Gb) shows a similar open cross-section but instead showing the radiopaque features of the implant delivery system 1100 (e.g., the catheter 1140, the delivery wire 600, and the implant 100) that a care provider would visualize during delivery of the implant under radiographic imaging and/or fluoroscopy.

FIGS. 13Aa-13Ab show the implant delivery system 1100 positioning the implant 100 (hidden from view) within the catheter 1140 adjacent the aneurysm 7 in a state prior to deployment of the implant 100. As shown, the catheter 1140 can include a distal radiopaque marker 1182 adjacent its distal end 1102 and a proximal radiopaque marker 1181 spaced proximally from the distal end 1102. Furthermore, as shown, in the pre-deployment state the distal end of the radiopaque coil 680 and at least one distal radiopaque marker 182 of the implant 100 can be substantially aligned longitudinally with one another and can be proximal of the distal radiopaque marker 1182 of the catheter 1140 (indicating the distal ends 102 and 602 of the implant 100 and the delivery wire 600, respectively, are within the lumen 1144 of the catheter 1140).

FIGS. 13Ba-13Bb show the implant delivery system 1100 in a state where the distal end of the radiopaque coil 680, the at least one distal radiopaque marker 182 of the implant 100, and the distal radiopaque marker 1182 of the catheter 1140 are substantially aligned with one another. Such positioning can be attained by moving the catheter 1140 proximally relative to the delivery wire 600 (and thus the implant 100) and/or moving the delivery wire 600 distally relative to the catheter 1140. In such positioning, the distal ends 102 and 602 of the implant 100 and the delivery wire 600, respectively, can be located within the lumen 1144 and adjacent the exit port 1112 of the catheter 1140.

FIGS. 13Ca-13Cb show the implant delivery system 1100 in a state where the implant 100 is partially deployed (e.g., about 25% deployed) within the vessel 5 and in a position adjacent but distal to the aneurysm 7. As shown, the distal end of the radiopaque coil 680 and the at least one distal radiopaque marker 182 of the implant 100 are both distal of the distal radiopaque marker 1182 of the catheter 1140. Also shown, the distal end of the radiopaque coil 680 is distal of the at least one distal radiopaque marker 182 of the implant 100. Such positioning can be attained by moving the catheter 1140 proximally relative to the delivery wire 600 (and thus the implant 100) and/or moving the delivery wire 600 distally relative to the catheter 1140 more so than shown in FIGS. 13Ba-13Bb. In such positioning, the distal end 602 of the delivery wire 600 can extend distal of the distal end 102 of the implant 100 and distal and out of the exit port 1112 of the catheter 1140. Furthermore, the distal end 102 and at least a portion of the implant 100 can extend distal and out of the exit port 1112 of the catheter 1140 and expand radially outward against the internal wall of the vessel 5.

FIGS. 13Da-13Db show the implant delivery system 1100 in another state where the implant 100 is partially deployed (e.g., about 50% deployed) and where more of the implant 100 is deployed within the vessel 5 than in the partially deployed state shown in FIGS. 13Ca-13Cb. The implant 100 in this partially deployed state extends at least partially across the aneurysm 7. As shown, the distal end of the radiopaque coil 680 and the at least one distal radiopaque marker 182 of the implant 100 are both more distal of the distal radiopaque marker 1182 of the catheter 1140 than shown in FIGS. 13Ca-13Cb. Also shown, the distal end of the radiopaque coil 680 is more distal of the at least one distal radiopaque marker 182 of the implant 100 than shown in FIGS. 13Ca-13Cb. Such positioning can be attained by moving the catheter 1140 proximally relative to the delivery wire 600 (and thus the implant 100) and/or moving the delivery wire 600 distally relative to the catheter 1140 more so than shown in FIGS. 13Ca-13Cb. In such positioning, the distal end 602 of the delivery wire 600 can extend further distal of the distal end 102 of the implant 100 and further distal and out of the exit port 1112 of the catheter 1140 than shown in FIG. 13C. Furthermore, more of the implant 100 can extend distal and out of the exit port 1112 of the catheter 1140 and expand radially outward against the internal wall of the vessel 5 where surrounded thereby.

FIGS. 13Ea-13Eb show the implant delivery system 1100 in another state where the implant 100 is partially deployed (e.g., about 75% deployed) and at the resheathable limit. In other words, FIGS. 13Ea-13Eb show the implant delivery system 1100 in a position at which further deployment of the implant 100 can cause the implant 100 to fully deploy within the vessel 5. As shown, more of the implant 100 is deployed within the vessel 5 than in the partially deployed state shown in FIGS. 13Da-13Db. The implant 100 in this partially deployed and resheathable limit state extends further across the aneurysm 7 than shown in FIGS. 13Da-13Db. As shown, the distal end of the radiopaque coil 680 and the at least one distal radiopaque marker 182 of the implant 100 are both more distal of the distal radiopaque marker 1182 of the catheter 1140 than shown in FIGS. 13Da-13Db. Also shown, the distal end of the radiopaque coil 680 can be more distal of the at least one distal radiopaque marker 182 of the implant 100 than shown in FIGS. 13Da-13Db. Further still shown, the proximal end of the radiopaque coil 680 can substantially align with the distal radiopaque marker 1182 of the catheter 1140, which can advantageously indicate that the implant 100 is at the resheathable limit. Such positioning can be attained by moving the catheter 1140 proximally relative to the delivery wire 600 (and thus the implant 100) and/or moving the delivery wire 600 distally relative to the catheter 1140 more so than shown in FIGS. 13Da-13Db. In such positioning, the distal end 602 of the delivery wire 600 can extend further distal of the distal end 102 of the implant 100 and further distal and out of the exit port 1112 of the catheter 1140 than shown in FIGS. 13Da-13Db. Furthermore, more of the implant 100 can extend distal and out of the exit port 1112 of the catheter 1140 and expand radially outward against the internal wall of the vessel 5 where surrounded thereby.

FIGS. 13Fa-13Fb show the implant delivery system 1100 in a state where the implant 100 is nearly fully deployed (e.g., about 95% deployed) and, in some implementations, past the resheathable limit. As shown, more of the implant 100 is deployed within the vessel 5 than in the partially deployed state shown in FIGS. 13Ea-13Eb. The implant 100 in this nearly fully deployed state extends across the aneurysm 7. As shown, the distal end of the radiopaque coil 680 and the at least one distal radiopaque marker 182 of the implant 100 are both more distal of the distal radiopaque marker 1182 of the catheter 1140 than shown in FIGS. 13Ea-13Eb. Also shown, the distal end of the radiopaque coil 680 can be more distal of the at least one distal radiopaque marker 182 of the implant 100 than shown in FIGS. 13Ea-13Eb. Further still shown, the proximal end of the radiopaque coil 680 can be distal of the distal radiopaque marker 1182 of the catheter 1140 and the at least one proximal radiopaque markers 181 of the implant can be substantially aligned with the distal radiopaque marker 1182 of the catheter 1140, which can advantageously indicate that the implant 100 is past the resheathable limit. Such positioning can be attained by moving the catheter 1140 proximally relative to the delivery wire 600 (and thus the implant 100) and/or moving the delivery wire 600 distally relative to the catheter 1140 more so than shown in FIGS. 13Ea-13Eb. In such positioning, the distal end 602 of the delivery wire 600 can extend further distal of the distal end 102 of the implant 100 and further distal and out of the exit port 1112 of the catheter 1140 than shown in FIGS. 13Ea-13Eb. Furthermore, more of the implant 100 can extend distal and out of the exit port 1112 of the catheter 1140 and expand radially outward against the internal wall of the vessel 5 where surrounded thereby.

FIGS. 13Ga-13Gb show the implant delivery system 1100 in a state where the implant 100 is fully deployed within the vessel 5. As shown, more of the implant 100 is deployed within the vessel 5 than in the nearly fully deployed state shown in FIGS. 13Fa-13Fb. The implant 100 in this fully deployed state extends across the aneurysm 7 (e.g., at least partially distal and at least partially proximal of the aneurysm 7) and is no longer connected to the delivery wire 600. As shown, the distal end of the radiopaque coil 680 and the at least one distal radiopaque marker 182 of the implant 100 are both more distal of the distal radiopaque marker 1182 of the catheter 1140 than shown in FIGS. 13Fa-13Fb. Also shown, the distal end of the radiopaque coil 680 can be more distal of the at least one distal radiopaque marker 182 of the implant 100 than shown in FIGS. 13Fa-13Fb. Further still shown, the at least one proximal radiopaque marker 181 of the implant 100 can be distal of the distal radiopaque marker 1182 of the catheter 1140, which can advantageously indicate that the implant 100 is fully deployed and no longer connected to the delivery wire 600. Such positioning can be attained by moving the catheter 1140 proximally relative to the delivery wire 600 (and thus the implant 100) and/or moving the delivery wire 600 distally relative to the catheter 1140 more so than shown in FIGS. 13Fa-13Fb. In such positioning, the distal end 602 of the delivery wire 600 can extend further distal of the distal end 102 of the implant 100 and further distal and out of the exit port 1112 of the catheter 1140 than shown in FIGS. 13Fa-13Fb. Furthermore, none of the implant 100 can be contained by the catheter 1140 and all of the implant 100 including its proximal end 101 can expand radially outward against the internal wall of the vessel 5 where surrounded thereby.

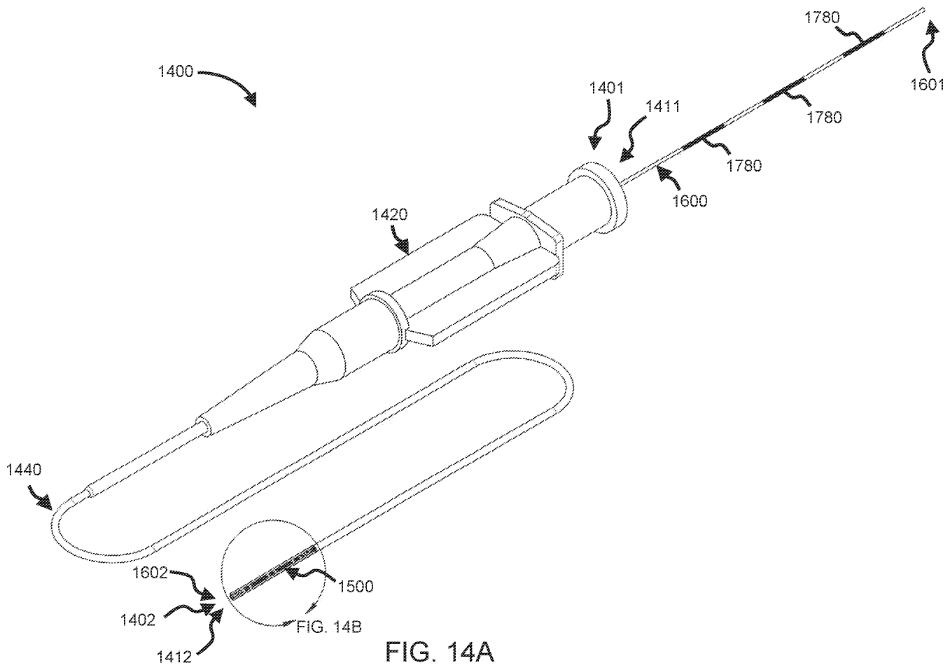

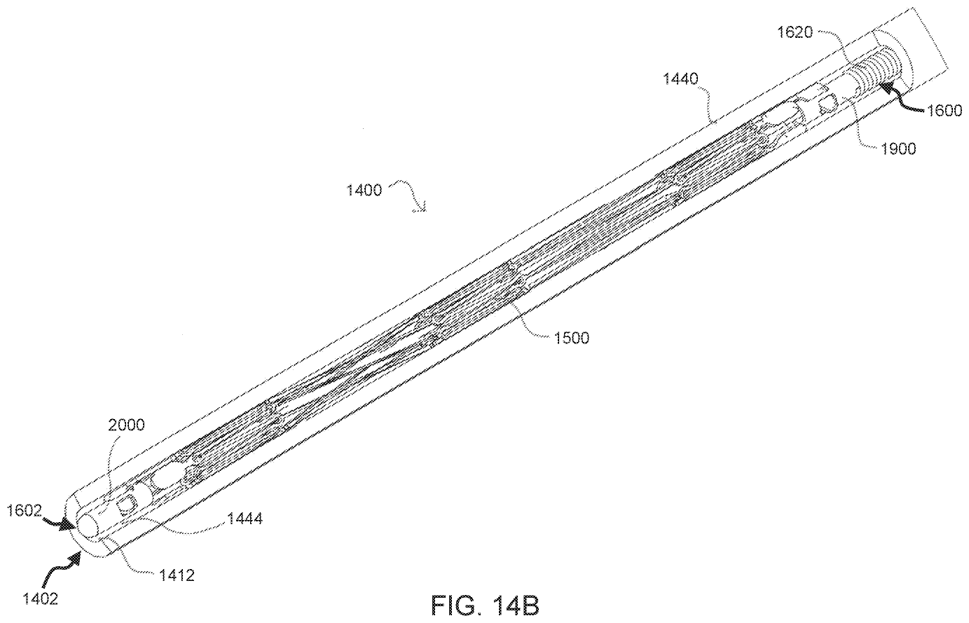

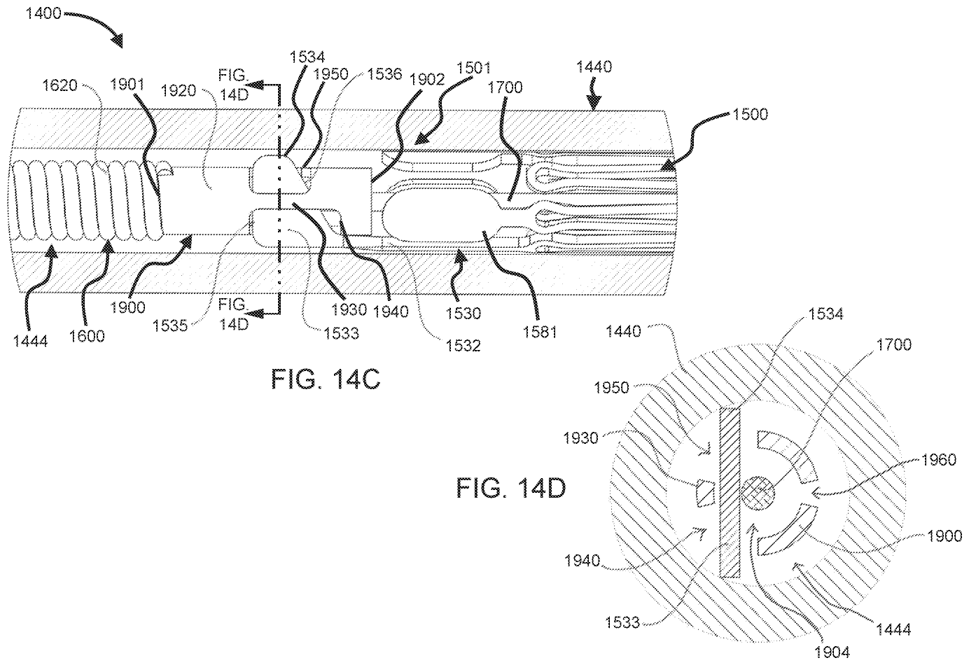

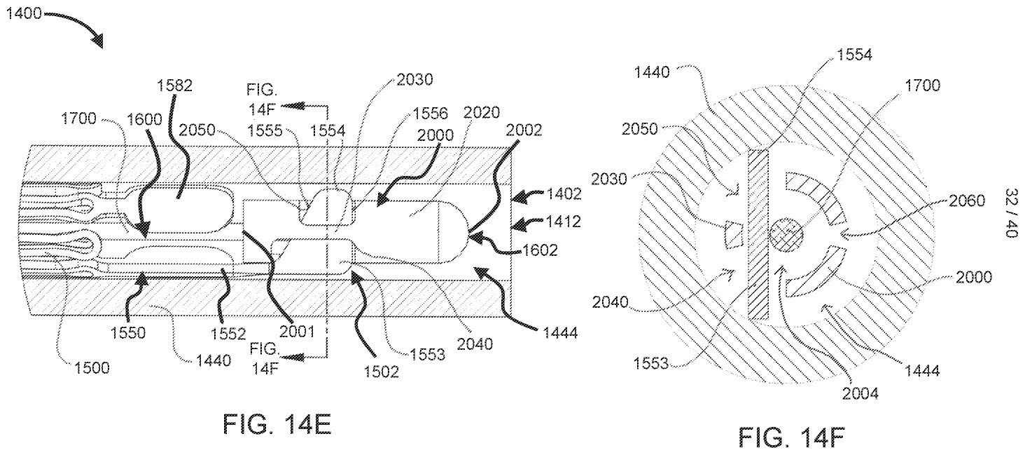

FIGS. 14A-14F illustrate another implementation of an intraluminal implant delivery system 1400 in accordance with some aspects of this disclosure. FIG. 14A shows a perspective view of the implant delivery system 1400. FIG. 14B shows a close-up perspective view of the distal end of the implant delivery system 1400 with a portion of the wall of the catheter 1440 removed from view. FIGS. 14C-14D a side and related cross-sectional view of the proximal end 1501 of the implant 1500 in a collapsed configuration within the implant delivery system 1400. FIGS. 14E-14F show a side and related cross-sectional view of the distal end 1502 of the implant 1500 in a collapsed configuration within the implant delivery system 1400.

The intraluminal implant delivery system 1400 can be similar to or the same as the implant delivery system 1100 in some or many respects and/or include any of the functionality of the implant delivery system 1100. For example, the implant delivery system 1400 can have a catheter 1440 with a generally tubular body with a lumen 1444 extending between an access port 1411 at its proximal end 1401 and an exit port 1412 at its distal end 1402 similar to or the same as the catheter 1140 with proximal end 1101, access port 1111, exit port 1112, distal end 1102, and lumen 1144. Furthermore, the catheter 1440 can include a hub 1420 adjacent the access port 1411 at its proximal end 1401 similar or the same as the hub 1120 of catheter 1140. The implant delivery system 1400 can also be configured to delivery an implant 1500 collapsed about a delivery wire 1600 similar to the implant 100 and the delivery wire 600 of implant delivery system 1100, although the implant 1500 and the delivery wire 1600 can be configured differently. For example and as shown, the delivery wire 1600 can extend generally longitudinally between its proximal end 1601 and its distal end 1602 similar to or the same as the delivery wire 600. The delivery wire 1600 can also have a core wire 1700 with one or more markers 1780 similar to or the same as the core wire 700 with markers 780 of the delivery wire 600. The delivery wire 1600 can differ from the delivery wire 600 in that it can have a proximal coil 1620, a proximal coupler 1900, and in some implementations a distal coupler 2000, the proximal coupler 1900 and the distal coupler 2000 configured to interact with the implant 1500 for delivery thereof. Similar to the delivery wire 600, the delivery wire 700 can be configured to travel through the catheter 1440 and to deliver the implant 1500.

The proximal coupler 1900 of the delivery wire 1600 can have a generally tubular body 1920 with a lumen 1904 extending between its proximal end 1901 and its distal end 1902. As shown in at least FIGS. 14C-14D, the proximal coupler 1900 can have one or more windows configured to receive and releasably connect with at least a portion of the implant 1500 for delivery thereof. For example, the proximal coupler 1900 can have a first window 1940, a second window 1950, and a third window 1960. The first window 1940 and the second window 1950 can at least partially align with one another across a side of the proximal coupler 1900 and be separated from one another by a strut 1930. The third window 1960 can provide a through hole via which the proximal coupler 1900 can be attached (e.g., welded) to the core wire 1700 of the delivery wire 1600.

The distal coupler 2000 of the delivery wire 1600, when included, can have a generally tubular body 2020 with a lumen 2004 extending at least partially from its proximal end 2001 to its distal end 2002 and with a closed rounded distal end 2002. As shown in at least FIGS. 14E-14F, the distal coupler 2000 can have one or more windows configured to receive and releasably connect with at least a portion of the implant 1500 for delivery thereof. For example, the distal coupler 2000 can have a first window 2040, a second window 2050, and a third window 2060. The first window 2040 and the second window 2050 can at least partially align with one another across a side of the distal coupler 2000 and be separated from one another by a strut 2030. The third window 2060 can provide a through hole via which the distal coupler 2000 can be attached (e.g., welded) to the core wire 1700 of the delivery wire 1600.

The implant 1500 can be the same as or similar to the implants 100, 400, and/or 500 in some or many respects and/or include any of the functionality of the implants 100, 400, and/or 500. For example, the implant 1500 can include rings 1541 comprising a plurality of ring struts 1542 that join at a plurality of proximal apexes 1543 and a plurality of distal apexes 1544 joined by a plurality of linking struts 1545 similar or the same as the rings 141, 121, 161, the plurality of ring struts 142, 122, 162, the plurality of proximal apexes 143, 123, 163, the plurality of distal apexes 144, 124, 164, and the plurality of linking struts 145 of the implant 100. The implant 1500 can differ from the implants 100, 400, and/or 500 in how it is configured to releasably connect with its associated delivery wire 1600. As shown in at least FIGS. 14C-14D, the implant 1500 can have a proximally extending strut 1530 configured to releasably connect/interact with the proximal coupler 1900 of the delivery wire 1600 for delivery of the implant 1500. The proximally extending strut 1530 can have a neck portion 1532 and a proximal flag 1533, with the proximal flag having a proximal surface 1535, a free end 1534, and a distal surface 1536. The neck portion 1532 can be configured to extend proximally past one or more proximal radiopaque markers 1581 that extend proximally from the implant 1500 when the implant 1500 is in its collapsed state within the catheter 1444 and about the delivery wire 1600. The proximal flag 1533 can extend proximally from the neck portion 1532 and be configured to releasably connect with the proximal coupler 1900 of the delivery wire 1600. For this, the free end 1534 of the proximal flag 1533 can insert within the first window 1940 and the second window 1950 of the proximal coupler 1900 such that the proximal flag 1533 at least partially extends through the first window 1940 and the second window 1950. Also, the proximal surface 1535 and/or distal surface 1536 can be angled relative to the longitudinal axis of the implant 1500 as shown to aid in the connection and interaction between the proximal flag 1533 and the proximal coupler 1900. The strut 1930 of the proximal coupler 1900 can prevent the proximal flag 1533 from slipping radially outward from the proximal coupler 1900, and the lumen 1444 of the catheter 1440 can prevent the proximal flag 1533 from slipping out from the first window 1940 and the second window 1950 while the proximal coupler 1900 remains inside the lumen 1444 of the catheter 1440. Furthermore, the proximal surface 1535 and the distal surface 1536 of the proximal flag 1533 can interact with corresponding proximal and distal surfaces of the first window 1940 and the second window 1950 for advancement or retraction of the implant 1500 relative to the catheter 1440 when the delivery wire 1600 is correspondingly advanced or retracted relative to the catheter 1440.

If the implant 1500 has sufficient pushability, the implant 1500 and the delivery wire 1600 may releasably connect via the proximal flag 1533 and the proximal coupler 1900 alone. If the implant 1500 does not have sufficient pushability, the implant 1500 and the delivery wire 1600 can, in addition to the releasably connection via the proximal flag 1533 and the proximal coupler 1900, releasably connect in a similar fashion via a distal flag 1553 of a distally extending strut 1550 of the implant 1500 and the distal coupler 2000. As shown in at least FIGS. 14E-14F, the implant 1500 can have the distally extending strut 1550 configured to releasably connect/interact with the distal coupler 2000 of the delivery wire 1600 for delivery of the implant 1500. The proximally extending strut 1550 can have a neck portion 1552 and the distal flag 1553, with the distal flag having a proximal surface 1555, a free end 1554, and a distal surface 1556. The neck portion 1552 can be configured to extend distally past one or more proximal radiopaque markers 1582 that extend distally from the implant 1500 when the implant 1500 is in its collapsed state within the catheter 1444 and about the delivery wire 1600. The distal flag 1553 can extend distally from the neck portion 1552 and be configured to releasably connect with the distal coupler 2000 of the delivery wire 1600. For this, the free end 1554 of the distal flag 1553 can insert within the first window 2040 and the second window 2050 of the distal coupler 2000 such that the distal flag 1553 at least partially extends through the first window 2040 and the second window 2050. Also, the proximal surface 1555 and/or distal surface 1556 can be angled relative to the longitudinal axis of the implant 1500 as shown to aid in the connection and interaction between the distal flag 1553 and the distal coupler 2000. The strut 2030 of the distal coupler 2000 can prevent the distal flag 1553 from slipping radially outward from the distal coupler 2000, and the lumen 1444 of the catheter 1440 can prevent the distal flag 1553 from slipping out from the first window 2040 and the second window 2050 while the distal coupler 2000 remains inside the lumen 1444 of the catheter 1440. Furthermore, the proximal surface 1555 and the distal surface 1556 of the distal flag 1553 can interact with corresponding proximal and distal surfaces of the first window 2040 and the second window 2050 for advancement or retraction of the implant 1500 relative to the catheter 1440 when the delivery wire 1600 is correspondingly advanced or retracted relative to the catheter 1440. If the implant 1500 has sufficient pushability to advance and/or retract through the catheter 1440 via the connection between the proximal flag 1533 and the proximal coupler 1900 alone, the implant 1500 may not have the distally extending strut 1550 and the delivery wire 1600 may not have the distal coupler 2000.

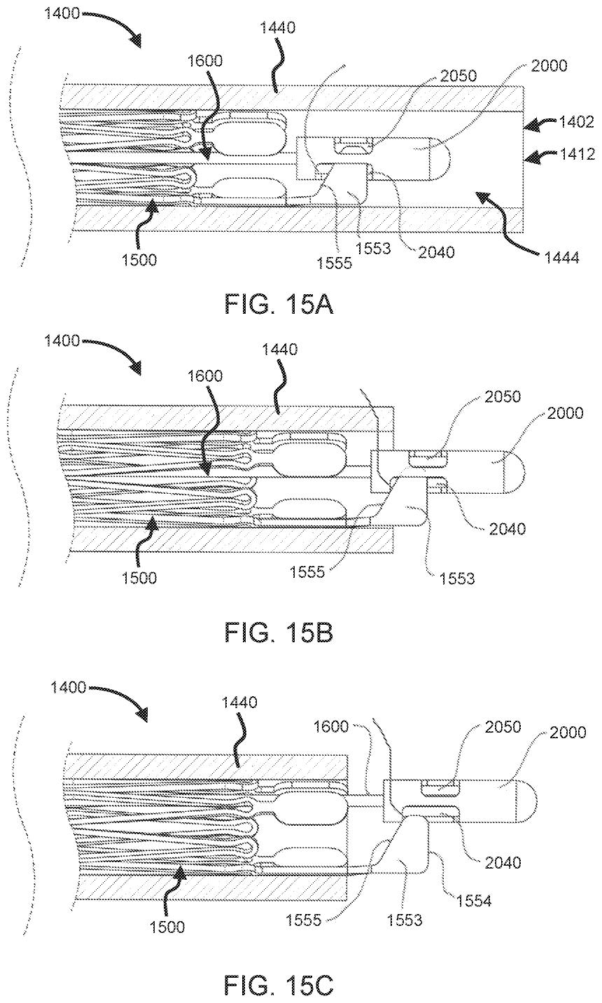

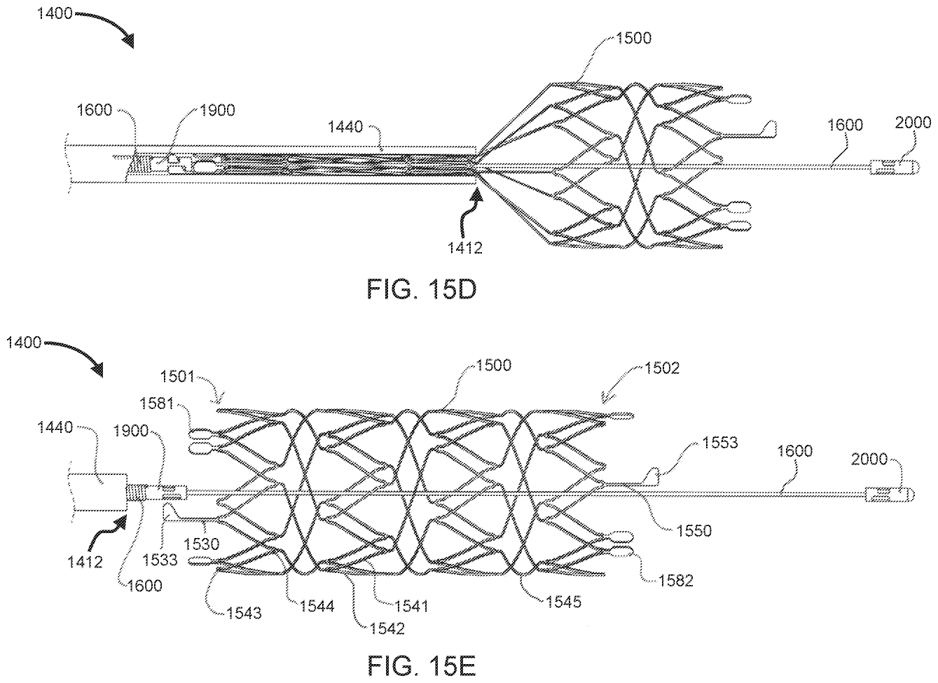

FIGS. 15A-15E illustrate delivery of the implant 1500 via the delivery wire 1600 and catheter 1440 in accordance with some aspects of this disclosure. FIG. 15A shows the implant 1500 collapsed about the delivery wire 1600 within the lumen 1444 of the catheter 1440 with the distal flag 1550 inserted within the first window 2040 and the second window 2050 of the distal coupler 2000 and adjacent the exit port 1412 of the catheter 1440. This can be the relative positioning used during advancement of the implant delivery system 1400 within the subject 1 to the desired site of implantation. FIG. 15B shows the implant 1500 still collapsed about the delivery wire 1600 with a portion of the implant 1500 extending distally from the exit port 1412 at the distal end 1402 of the catheter 1140. Also shown is at least a portion of the delivery wire 1600 extending distally from the exit port 1412 of the catheter 1440, and the distal flag 1550 beginning to disconnect from the distal coupler 2000. To attain such partial extension of the implant 1500 and delivery wire 1600 and such partial disconnection therebetween, the delivery wire 1600 can be moved distally relative to the catheter 1440 and/or the catheter 1440 can be moved proximally relative to the delivery wire 1600. In some implementations, it is advantageous to simultaneously move the catheter 1440 proximally and the delivery wire 1600 distally during implant 1500 deployment. In the partially extended state shown in FIG. 15B, relative movement between the delivery wire 1600 and the catheter 1440 can either withdraw the implant 1500 back into the catheter 1440 and fully reconnect the distal flag 1550 and the distal coupler 2000 or continue on and eventually release the flag 1550 from the distal coupler 2000 as shown in FIG. 15C. In other words, once the distal flag 1550 is extended distally completely out the exit port 1412 from within the lumen 1444 of the catheter 1440, the distal flag 1553 can disconnect from the distal coupler 2000. FIG. 15D shows the implant 1500 partially expanded with a portion of the implant 1500 extending distally from the exit port 1412 at the distal end 1402 of the catheter 1440. Also shown is at least a portion of the delivery wire 1600 extending distally from the exit port 1412 of the catheter 1440. To attain such partial expansion of the implant 1500, the delivery wire 1600 can be moved distally relative to the catheter 1440 and/or the catheter 1440 can be moved proximally relative to the delivery wire 1600 more so than shown in FIG. 15C. In the partially expanded state (which can also be referred to herein as a partially deployed state) shown in FIG. 15D, relative movement between the delivery wire 1600 and the catheter 1440 can either withdraw the implant 1500 back into the catheter 1440 or continue on and eventually release the implant 1500 altogether from the exit port 1412 of the catheter 1440 and from the delivery wire 1600 as shown in FIG. 15E. Such relative movement can be attained by the releasable connection between the proximal flag 1533 and the proximal coupler 1900 as discussed above. The implant delivery system 1400 can be configured such that the implant 1500 can be able to be withdrawn within the lumen 1444 of the catheter 1440 (e.g., for adjusting position or removing from the subject 1) while the coupler 1900 of the delivery wire 1600 stays within the lumen 1444 of the catheter 1440. In implementations where the implant 1500 is without the distally extending strut 1550 with the distal flag 1553 the delivery wire 1600 is without the distal coupler 2000, the implant 1500 can be deployed from the catheter 1440 via the interaction between the proximal flag 1533 and the proximal coupler 1900 as discussed above.

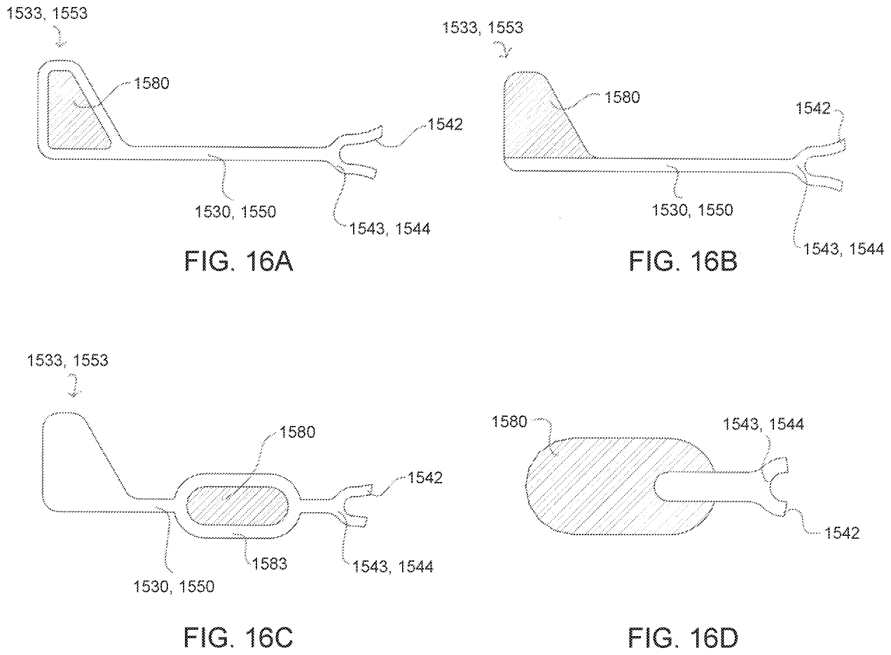

FIGS. 16A-16D illustrate various implementations of radiopaque markers of the implant 1500 in accordance with some aspects of this disclosure. Each of FIGS. 16A-16D show a proximal and/or distal end of the implant 1500 where a plurality of ring struts 1542 connect at either one of a plurality of proximal apexes 1543 or one of a plurality of distal apexes 1544, respectively. FIG. 16A shows an implementation wherein the proximal flag 1533 and/or distal flag 1553 includes a radiopaque material 1580 (e.g., within a portion of the flag), with such proximal flag 1533 extending from a corresponding proximally extending strut 1530 or such distal flag 1553 extending from a corresponding distally extending strut 1550. FIG. 16B shows an implementation wherein the proximal flag 1533 and/or distal flag 1553 comprises a radiopaque material 1580, with such proximal flag 1533 extending from a corresponding proximally extending strut 1530 or such distal flag 1553 extending from a corresponding distally extending strut 1550. FIG. 16C shows an implementation wherein the proximally extending strut 1530 and/or the distally extending strut 1550 includes an islet 1583 incorporating a radiopaque material 1580. FIG. 16D shows an implementation of the proximal radiopaque markers 1581 and/or the distal radiopaque markers 1582 where such markers include a radiopaque material 1580

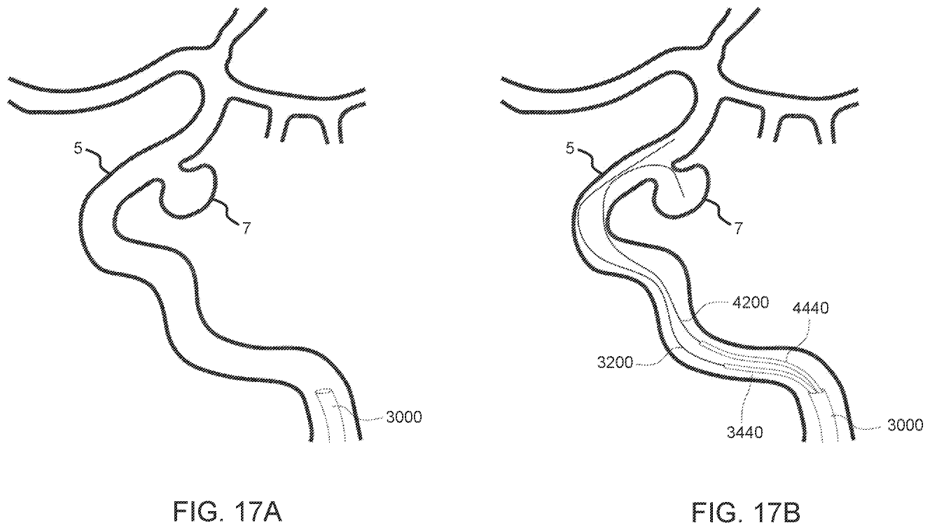

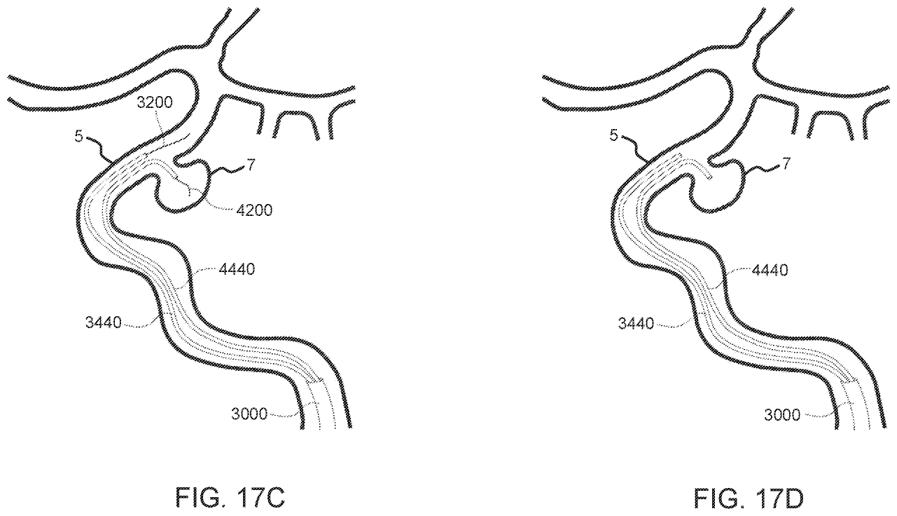

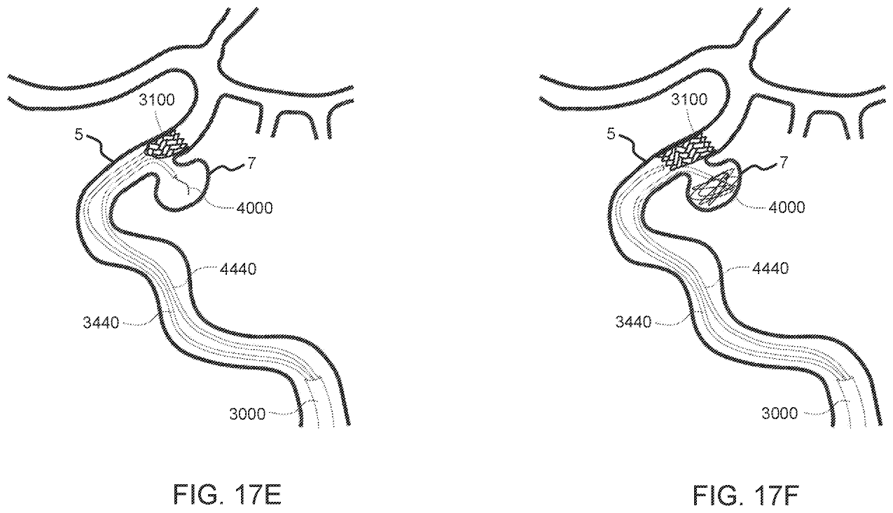

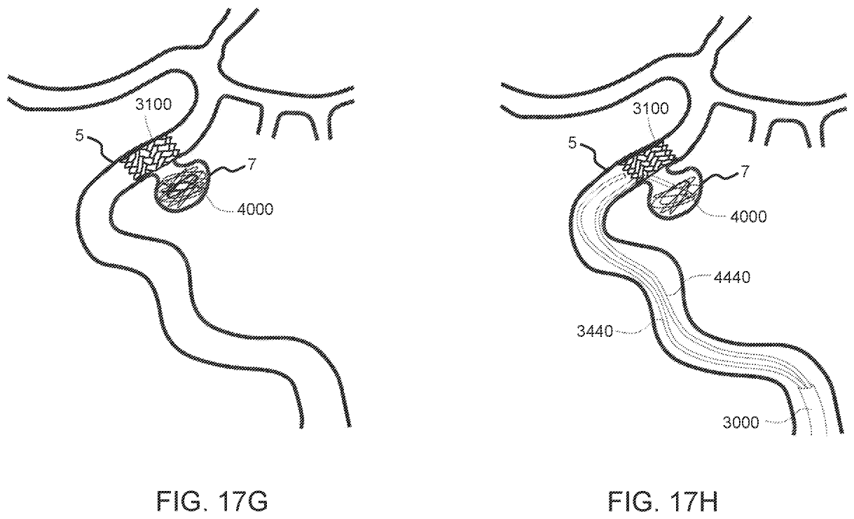

FIGS. 17A-17G illustrate a method of treating an aneurysm 7 of a vessel 5 in accordance with some aspects of this disclosure. The method described with respect to FIGS. 17A-17G is intended to be a general, non-limiting method for treating an aneurysm using any of the implant delivery systems and/or components thereof described herein, such as the implant delivery system 1100, the implant delivery system 1400, or variants thereof. FIGS. 17A-17G show the general progression of a method of deploying coil(s) 4000 as well as the implant 3100. FIG. 17A shows use of a catheter 3000 to establish a path to the aneurysm 7. FIG. 17B shows use of guidewires 3200, 4200 to help guide catheters 3440, 4440 for both the implant 3100 and the coil(s) 4000, respectively, to the aneurysm 7 (although in some implementations, use of such guidewires may not be necessary or required). FIG. 17C shows the placement of the coil catheter 4440 in the aneurysm 7 for delivering the coil(s) 4000 and the implant catheter 3440 in the vessel 5 adjacent to the aneurysm 7 for placing the implant 3100. FIG. 17D shows the catheters 3440, 4440 upon removal of the guidewires 3200, 4200. FIG. 17E shows the deployment of the implant 3100 and the coil(s) 4000 from the respective catheters 3440, 4440. FIG. 17F shows the expanded implant 3100 upon deployment and the aneurysm 7 upon being packed with coil(s) 4000. FIG. 17G shows the packed aneurysm 7 after the catheters 3440, 4440 are retracted and the implant 3100 implanted within the vessel 5 adjacent the aneurysm 7. FIG. 17H shows an alternative of the method wherein the implant 3100 is first deployed within the vessel 5 adjacent the aneurysm 7 and the coil catheter 4440 extends through the implant 3100 and into the aneurysm 7 for the deployment of the coil(s) 4000. Such alternative can advantageously aid in retention of the coil(s) 4000 within the aneurysm during and after their deployment.

Alternatively, or in addition, the catheter 4440 can release a two-step in situ gel with a secondary chemical trigger to fill an aneurysm sac or arteriovenous malformation. For example, the first step may comprise injecting a shear-thinning gel (e.g., Bingham plastic like liquid, graft or copolymers including a phenylboronic group for glucose interaction, polyvinyl alcohol (PVA), polyethylenimine (PEI), gelatin, polyethylene glycol (PEG), PolyAlginate, Hyaluronic acid, and glycosaminoglycans (GAG), etc.) into the aneurysm sac with or without coils. The second step may comprise cross-linking by injecting a benign metabolite (e.g., glucose, fructose, etc.) into the viscous gel precursor liquid. In some implementations, salt concentration, calcium ion concentration, ethanol, riboflavin, and other metabolic properties can be used in lieu of or in addition to glucose and/or fructose.

In some implementations, the catheter 4440 can release a two-step in situ gel with physical trigger to fill an aneurysm sac or arteriovenous malformation. For example, the first step may comprise injecting a shear-thinning gel (e.g., Bingham plastic like liquid, Pluronic, PNIPPAM plus Pluronic, etc.) into the aneurysm sac with or without coils. The second step may comprise a physical crosslinking step, for example physical crosslinking by injecting benign high/low temperature saline into the viscous gel precursor liquid. Alternatively, body temperature may be sufficient to crosslink the gel.

In some implementations, a two-step in situ gel may be coated on or incorporated into aneurysm coils prior to deployment. The precoated coil may be deployed containing the shear-thinning plastic like liquid including Bingham, Pluronic, PNIPPAM plus Pluronic and other like polymers or viscous gel precursor liquid including a graft or copolymers including a phenylboronic group for glucose interaction, polyvinyl alcohol (PVA), polyethylenimine (PEI), gelatin, polyethylene glycol (PEG), PolyAlginate, Hyaluronic acid, and glycosaminoglycans (GAG). The secondary chemical or physical crosslink can be induced as described elsewhere herein.

In some implementations, the implants described herein can be designed only to assist in deployment of the coil(s) 4000 and may be removed after packing of the coil(s) 4000 in the aneurysm 7. Such an implant may optionally then be replaced by a permanent implant, which may be of substantially similar design or of a different design. Alternatively and as described herein, the implants may serve as a permanent implant which remains in place after deployment and packing of the aneurysm 7 with coil(s) 4000.

In some implementations, particularly for treatment of ICAS, an implant as described herein can be deployed in a vessel such that it covers plaque in the vessel.

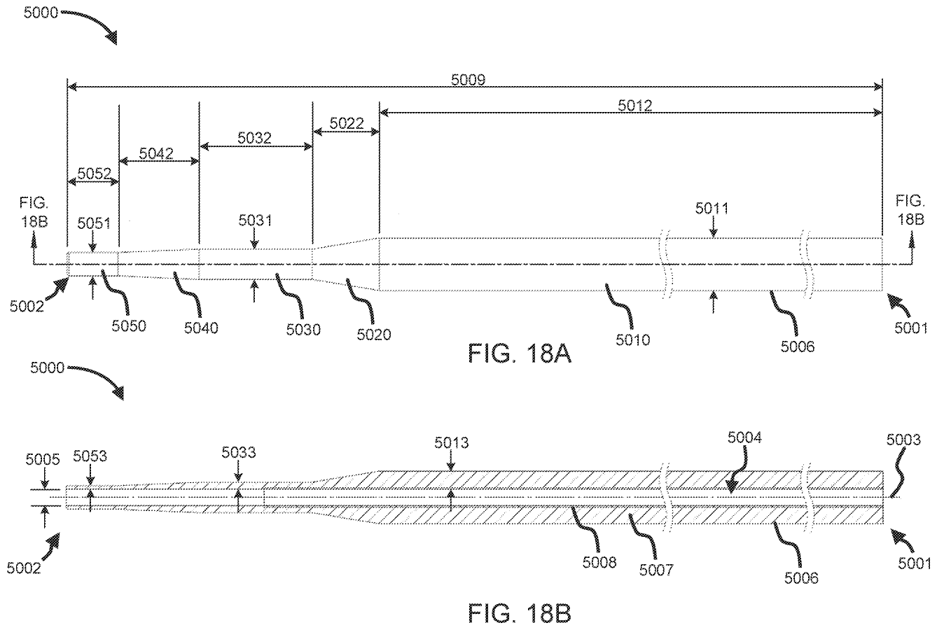

FIGS. 18A-18B illustrate an introducer sheath 5000 in accordance with some aspects of this disclosure. FIG. 18A shows a side view and FIG. 18B an associated cross-sectional side view of the introducer sheath 5000. The introducer sheath 5000 can be configured to aid in insertion of an implant as described herein, such as the implants 100, 400, 500, or 1500, in its collapsed state about a delivery wire as described herein, such as the delivery wires 600 or 1600, through the proximal end of a catheter as described herein, such as the catheters 1140 or 1440, and/or through the proximal end of a hemostatic valve attached to the proximal end of a catheter as described herein. As shown in FIGS. 18A-18B, the introducer sheath 5000 can have a generally tubular body 5006 having a proximal end 5001, a distal end 5002, a length 5009 and a longitudinal axis 5003 extending between the proximal end 5001 and the distal end 5002, and an inner diameter 5005 defining a lumen 5004. The introducer sheath 5000 can include one or more substantially constant diameter sections and one or more tapered sections to produce an introducer sheath 5000 having a greater outer diameter at its proximal end 5001 than at its distal end 5002. For example and as shown in FIGS. 18A-18B, the introducer sheath 5000 can include a first constant diameter section 5010 having an outer diameter 5011 and a length 5012, a first tapered section 5020 having a length 5022, a second constant diameter section 5030 having an outer diameter 5031 and a length 5032, a second tapered section 5040 having a length 5042, and a third constant diameter section 5050 having an outer diameter 5051 and a length 5052. As shown, the first constant diameter section 5010 can extend distally from the proximal end 5001 of the introducer sheath 5000, the first tapered section 5020 can extend distally from the first constant diameter section 5010, the second constant diameter section 5030 can extend distally from the first tapered section 5020, the second tapered section 5040 can extend distally from the second constant diameter section 5030, and the third constant diameter section 5050 can extend distally from the second tapered section 5040 and terminate at the distal end 5002 of the introducer sheath 5000. In some implementations, the distal end 5002 of the introducer sheath 5000 is rounded to facilitate engagement with, for example, a hemostatic valve or catheter. The introducer sheath 5000 can include a jacket 5007 and a liner 5008 disposed within the jacket along at least a portion of its length. For example, the liner 5008 can extend from the proximal end 5002 and distal along at least a portion of the jacket 5007 (e.g., from the proximal end 5002 to at least a portion of the second constant diameter section 5030). The jacket 5007 can comprise Grilamid TR55 LX. The liner 5008 can comprise PTFE, for example extruded or dip coated. When assembled, the liner 5008 and the jacket 5007 can produce an introducer sheath 5000 having a substantially constant inner diameter 5005 of the lumen 5004 as shown. The inner diameter 5005 of the lumen 5004 can be configured to receive therethrough an implant collapsed over a delivery wire and maintain the collapsed state of the implant over the delivery wire while the two are disposed within and/or travel through the introducer sheath 5000. In some implementations, the inner diameter 5005 can be between about 0.200 mm and about 0.600 mm, for example about 0.427 mm.

The introducer sheath 5000 can have a length 5009 of at least about 250 mm. In some implementations, the introducer sheath 5000 has a length 5009 of about 500.126 mm. In such implementations, the length 5012 of the first constant diameter section 5010 can be about 492.100 mm, the length 5022 of the first tapered section 5020 can be about 1.727 mm, the length 5032 of the second constant diameter section 5030 can be about 2.921 mm, the length 5042 of the second tapered section 5040 can be about 2.057 mm, and the length 5052 of the third constant diameter section 5050 can be about 1.321 mm. The introducer sheath 5000 can have a maximum outer diameter of about 3 mm or less. In some implementations, the introducer sheath 5000 has a maximum outer diameter of about 1.346 mm. In such implementations, the outer diameter 5011 of the first constant diameter section 5010 can be about 1.346 mm, the outer diameter 5031 of the second constant diameter section 5030 can be about 0.787 mm, and the outer diameter 5051 of the third constant diameter section 5050 can be about 0.597 mm. Furthermore, in such implementations the first tapered section 5020 can taper over its length 5022 from the outer diameter 5011 of the first constant diameter section to the outer diameter 5031 of the second constant diameter section, and the second tapered section 5040 can taper over its length 5042 from the outer diameter 5031 of the second constant diameter section to the outer diameter 5051 of the third constant diameter section. Given the substantially constant inner diameter 5005 of the introducer sheath 5000, further in such implementations the thickness 5013 (e.g., wall thickness) of the first constant diameter section 5010 can be about 0.460 mm (which can include the jacket 5007 and the liner 5008), the thickness 5033 of the second constant diameter section 5030 can be about 0.175 mm (which can include the jacket 5007 and the liner 5008), and the thickness 5053 of the third constant diameter section 5050 can be about 0.838 mm. Although exemplary lengths and diameters for the introducer sheath 5000 and its sections 5010, 5020, 5030, 5040, and 5050 have been provided, any of such lengths and/or diameters can be less than or greater than those given, and/or such lengths and/or diameters can scale as the introducer sheath is reduced or increased in length and/or diameter.

In an exemplary method of use, the implant 100 can be collapsed over the delivery wire 600 as described herein (e.g., with the one or more slots 931 of the hub 930 of the coupler 900 of the delivery wire 600 receiving the neck portion 126 of one or more proximally extending struts 125 of the implant 100) and disposed within the lumen 5004 of the introducer sheath 5000 such that the implant 100 stays in its collapsed state. This can be, for example, a shipping configuration of the delivery wire 600, the implant 100, and the introducer sheath 5000. To introduce the delivery wire 600 with the implant 100 collapsed therearound into the catheter 1140, the insertion sheath 5000 can be partially inserted into a proximal end of a hemostatic valve that is attached to the proximal end 1101 of the catheter 1140. The hemostatic valve can then be tightened and the system flushed via the hemostatic valve (e.g., until fluid exits the proximal end 5001 of the introducer sheath 5000). The hemostatic valve can then be loosened and the introducer sheath 5000 advanced further distally until its distal end 5002 seats against the hub 1120 of the catheter 1140. The hemostatic valve can then be re-tightened to secure the introducer sheath 5000 in place relative to the catheter 1140. The delivery wire 600 with the implant 100 collapsed therearound can then be advanced distally until the entire implant 100 enters the catheter 1140. The delivery wire 600 can be advanced distally until the distal-most marker 780 of the core wire 700 of the delivery wire 600 is adjacent to the proximal end 5001 of the introducer sheath 5000. The introducer sheath 5000 can then be removed by loosening the hemostatic valve, pinning the delivery wire 600, and pulling the introducer sheath 5000 proximally over the delivery wire 600. The delivery wire 600 can be advanced until the same distal-most marker 780 is adjacent the proximal end of the hemostatic valve. The position of the implant 100 within the catheter 1140 can be adjusted by moving the delivery wire 600 and the catheter 1140 relative to one another, and preferably under radiographic imaging and/or fluoroscopy deployed within a subject 1 as described herein.

Although the implants, devices, systems, and methods disclosed herein have been described with respect to the treatment of an aneurysm of a patient, such as a neurovascular aneurysm, and/or the treat of intracranial artery stenosis, such disclosure is non-limiting. The implants, devices, systems, and methods disclosed herein can be used in the treatment of other conditions of a patient and/or for stenting any vessel of a patient. For example, the implants, devices, systems, and methods disclosed herein can be utilized in and/or adapted for any situation where it is desired to implant a stent implant having thromboresistant properties. As another example, the implants, devices, systems, and methods disclosed herein can be utilized in and/or adapted for any situation where exact placement of the implant at the implantation site is desired. In another example, the implants, devices, systems, and methods disclosed herein can be utilized in and/or adapted for any situation where adjustment of an implant's placement after partial deployment in a vessel is desirable.

Features, materials, characteristics, or groups described in conjunction with a particular aspect, implementation, or example are to be understood to be applicable to any other aspect, implementation or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing implementations. The protection extends to any novel one, or any novel combination, of the features disclosed in this specification (including any accompanying claims, abstract and drawings), or to any novel one, or any novel combination, of the steps of any method or process so disclosed.

While certain implementations have been described, these implementations have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some implementations, the actual steps taken in the processes illustrated or disclosed may differ from those shown in the figures. Depending on the implementation, certain of the steps described above may be removed, others may be added. For example, the actual steps or order of steps taken in the disclosed processes may differ from those shown in the figure. Depending on the implementation, certain of the steps described above may be removed, others may be added. Furthermore, the features and attributes of the specific implementations disclosed above may be combined in different ways to form additional implementations, all of which fall within the scope of the present disclosure.

Although the present disclosure includes certain implementations, examples and applications, it will be understood by those skilled in the art that the present disclosure extends beyond the specifically disclosed implementations to other alternative implementations or uses and obvious modifications and equivalents thereof, including implementations which do not provide all of the features and advantages set forth herein. Accordingly, the scope of the present disclosure is not intended to be limited by the described implementations, and may be defined by claims as presented herein or as presented in the future.

Conditional language, such as “can,” “could,” “might,” or “may,” unless specifically stated otherwise, or otherwise understood within the context as used, is generally intended to convey that certain implementations include, while other implementations do not include, certain features, elements, or steps. Thus, such conditional language is not generally intended to imply that features, elements, or steps are in any way required for one or more implementations or that one or more implementations necessarily include logic for deciding, with or without user input or prompting, whether these features, elements, or steps are included or are to be performed in any particular implementation. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

1. A self-expanding thromboresistant intraluminal implant, the implant comprising:
a generally tubular frame comprising:
a proximal portion comprising a ring that extends along a circumference of the tubular frame, the ring comprising a plurality of ring struts, wherein adjacent pairs of ring struts join at a plurality of proximal apexes and a plurality of distal apexes to form a chevron pattern;
a distal portion comprising a ring that extends along the circumference of the tubular frame, the ring comprising a plurality of ring struts, wherein adjacent pairs of ring struts join at a plurality of proximal apexes and a plurality of distal apexes to form a chevron pattern; and
a central portion between the proximal portion and the distal portion, the central portion comprising:
a plurality of longitudinally spaced apart rings that extend along the circumference of the tubular frame, each ring of the plurality of rings comprising a plurality of rings struts, wherein adjacent pairs of ring struts join at a plurality of proximal apexes and a plurality of distal apexes to form a chevron pattern, and
a plurality of linking struts that extend at least partially along the circumference of the tubular frame, each linking strut of the plurality of linking struts connecting a distal apex of one ring of the plurality of rings to a proximal apex of an adjacent ring of the plurality of rings.

2. The intraluminal implant of claim 1, wherein each linking strut of the plurality of linking struts connect each one of the plurality of distal apexes of one ring of the plurality of rings of the central portion to each one of the plurality of proximal apexes of an adjacent ring of the plurality of rings of the central portion except for at each one of a plurality of distal apexes of a distal most ring of the central portion and except for at each one of a plurality of proximal apexes of a proximal most ring of the central portion such that the central portion does not comprise any free apexes.

3. The intraluminal implant of claim 2, wherein each distal apex of the plurality of distal apexes of the distal most ring of the central portion connects to a respective proximal apex of the plurality of proximal apexes of the ring of the distal portion, and wherein each proximal apex of the plurality of proximal apexes of the proximal most ring of the central portion connects to a respective distal apex of the plurality of distal apexes of the ring of the proximal portion.

4. The intraluminal implant of claim 1, wherein each distal apex of the plurality of distal apexes of the one ring of the plurality of rings of the central portion is rotationally offset from each proximal apex of the plurality of proximal apexes of the adjacent ring of the plurality of rings of the central portion such that at least a portion of each linking strut of the plurality of linking struts extends along a helical path at least partially around the circumference of the tubular frame.

5. The intraluminal implant of claim 4, wherein the at least a portion of each linking strut of the plurality of linking struts connecting each distal apex of the plurality of distal apexes of the one ring of the plurality of rings of the central portion to each proximal apex of the plurality of proximal apexes of the adjacent ring of the plurality of rings of the central portion extends along the helical path at least partially around the circumference of the tubular frame in a first helical direction, and wherein at least a portion of each linking strut of a plurality of linking struts connecting each distal apex of a plurality of distal apexes of the adjacent ring of the plurality of rings of the central portion to each proximal apex of a plurality of proximal apexes of another adjacent ring of the plurality of rings of the central portion extends along the helical path at least partially around the circumference of the tubular frame in a second helical direction that is generally opposite the first helical direction.

6. The intraluminal implant of claim 1, further comprising:
one or more generally proximally extending struts extending from a respective one or more proximal apex of the plurality of proximal apexes of the ring of the proximal portion, wherein each of the one or more generally proximally extending struts comprises a neck portion and a connection portion, the connection portion configured to connect to a radiopaque marker; and
one or more radiopaque markers configured to connect to the one or more generally proximally extending struts at the connection portion thereof.

7. (canceled)

8. The intraluminal implant of claim 1, further comprising:
one or more generally distally extending struts extending from a respective one or more distal apex of the plurality of distal apexes of the ring of the distal portion, wherein each of the one or more generally distally extending struts comprises a neck portion and a connection portion, the connection portion configured to connect to a radiopaque marker; and
one or more radiopaque markers configured to connect to the one or more generally distally extending struts at the connection portion thereof.

9. (canceled)

10. (canceled)

11. The intraluminal implant of claim 1, wherein the proximal portion flares radially outward in a proximal direction and/or wherein the distal portion flares radially outward in a distal direction.

12. (canceled)

13. The intraluminal implant of claim 1, wherein the plurality of linking struts do not overlap one another.

14. The intraluminal implant of claim 1, wherein the intraluminal implant is configured to have less malappositions between the intraluminal implant and an inner wall of a vessel in which it is deployed on an inside of a bend of the vessel than on an outside of the bend of the vessel.

15. (canceled)

16. (canceled)

17. (canceled)

18. (canceled)

19. (canceled)

20. (canceled)

21. (canceled)

22. The intraluminal implant of claim 1, wherein the implant does not include a graft, covering, or liner.

23. The intraluminal implant of claim 1, further comprising a heparin coating.

24. A self-expanding thromboresistant intraluminal implant, the implant comprising:
a generally tubular frame comprising a plurality of longitudinally spaced apart rings that extend along a circumference of the tubular frame, each ring of the plurality of rings comprising a plurality of rings struts, wherein adjacent pairs of ring struts join at a plurality of proximal apexes and a plurality of distal apexes to form a chevron pattern, and a plurality of linking struts that extend at least partially along the circumference of the tubular frame, each linking strut of the plurality of linking struts connecting a distal apex of one ring of the plurality of rings to a proximal apex of an adjacent ring of the plurality of rings;
wherein the tubular frame comprises a wall thickness of about 45 μm or less, and
wherein the implant comprises a heparin coating.

25. The intraluminal implant of claim 24, wherein the heparin coating has a thickness of about 30 nm or less.

26. The intraluminal implant of claim 24, wherein the heparin coating has a mass of about 1.0 μg or less.

27. The intraluminal implant of claim 24, wherein a ratio of a mass of the heparin coating to a total surface area of the implant is about 0.007 ug/mm² or more.

28. (canceled)

29. (canceled)

30. The intraluminal implant of claim 24, wherein a ratio of a thickness of the heparin coating to the wall thickness of the tubular frame is about 0.00016 or greater.

31. The intraluminal implant of claim 24, wherein a particle size equivalent to an entirety of the heparin coating is about 101 μm in diameter or less.

32. The intraluminal implant of claim 24, wherein a ratio of heparin activity of the heparin coating to the wall thickness of the tubular frame is about 0.30 pmol AT/cm²/μm or more.

33. (canceled)

34. (canceled)

35. (canceled)

36. The intraluminal implant of claim 24, wherein the implant does not include a graft, covering, or liner.
